SEQUENCE LISTING (8 sequences)

SEQ ID NO: 1 - DNA, 21 bp, Artificial Sequence
  FANCD2D forward primer
acctgttatgagcgtgaagtc

SEQ ID NO: 2 - DNA, 21 bp, Artificial Sequence
  FANCD2D reverse primer
gatgcaggactgtgcattaga

SEQ ID NO: 3 - DNA, 21 bp, Artificial Sequence
  EXD2 forward primer
ggtctggcctaaggtttcttc

SEQ ID NO: 4 - DNA, 19 bp, Artificial Sequence
  EXD2 reverse primer
cagttcacgctgggttctt

SEQ ID NO: 5 - DNA, 21 bp, Artificial Sequence
  XRCC forward primer
gcagtcttcctagtctcaact

SEQ ID NO: 6 - DNA, 22 bp, Artificial Sequence
  XRCC reverse primer
ttgcccttctaggagcttaatg

SEQ ID NO: 7 - DNA, 20 bp, Artificial Sequence
  exon 2 of SIK2
aataatcgataagtctcagc

SEQ ID NO: 8 - DNA, 20 bp, Artificial Sequence
  exon 4 of SIK2
gattttcagctttgaggtca

(19) United States
(12) Patent Application Publication
(10) Pub. No.: US 2022/0296583 A1
(43) Pub. Date: Sep. 22, 2022

(54) COMPOSITIONS AND METHODS FOR TREATMENT OF OVARIAN AND BREAST CANCER

(71) Applicant: Board of Regents, The University of Texas System, Austin, TX (US)

(72) Inventors: Zhen LU, Pearland, TX (US); Robert C. BAST, Jr., Houston, TX (US)

(21) Appl. No.: 17/584,116

(22) Filed: Jan. 25, 2022

Related U.S. Application Data
(63) Continuation-in-part of application No. PCT/US2021/038571, filed on Jun. 23, 2021.
(60) Provisional application No. 63/163,118, filed on Mar. 19, 2021.
(60) Provisional application No. 63/164,308, filed on Mar. 22, 2021.

(51) Int. Cl. A61K 31/4545; A61K 31/555; A61K 31/337; A61K 33/243; A61K 45/06; A61P 35/04

(52) U.S. Cl. CPC A61K 31/4545; A61K 31/555; A61K 31/337; A61K 33/243; A61K 45/06; A61P 35/04

(57) ABSTRACT

Provided are methods of treating cancer comprising administering to a patient in need thereof a salt-induced kinase 2 (SIK2) inhibitor and at least a first chemotherapeutic drug. Also provided are methods of increasing or enhancing apoptosis of cancer cells in a patient having cancer, comprising administering to the patient a therapeutically effective amount of a SIK2 inhibitor and at least a first chemotherapeutic drug. Also provided are methods of enhancing sensitivity of ovarian cancer cells to a chemotherapeutic drug or to combinations of chemotherapeutic drugs in a patient having ovarian cancer, comprising contacting the cells with a therapeutically effective amount of a SIK2 inhibitor and at least a first chemotherapeutic drug. A method of increasing or enhancing carboplatin-induced DNA damage in a patient having ovarian cancer, comprising administering to the patient a therapeutically effective amount of a SIK2 inhibitor and at least a first chemotherapeutic drug.

### CROSS REFERENCE TO RELATED APPLICATIONS

This application is a continuation-in-part of International Appl. No. PCT/US2021/038571, filed on Jun. 23, 2021, which claims priority to, and the benefit of, U.S. Application No. 63/163,118, filed Mar. 19, 2021, the entirety of which is incorporated by reference herein. This application also claims priority to, and the benefit of, U.S. Application No. 63/164,308, filed Mar. 22, 2021, the entirety of which is incorporated by reference herein.

### GOVERNMENT LICENSE RIGHTS

This invention was made with government support under Grant Contract No. P50 CA217685 awarded by the National Institutes of Health. The government has certain rights in the invention.

### INCORPORATION OF SEQUENCE LISTING

The sequence listing that is contained in the file named “MDA0065-201CIP-US_ST25,” which is 1.86 kilobytes as measured in Microsoft Windows operating system and was created on Jan. 25, 2022, is filed electronically herewith and incorporated herein by reference.

Recent studies indicate that DNA damage, aberrations in the DNA damage response and defects in DNA repair machinery play a major role in ovarian and triple-negative breast cancer (TNBC). DNA double-strand breaks (DSBs) are considered one of the most cytotoxic forms of DNA damage that can lead to mutation and trigger permanent growth arrest or cell death. The two main DSB repair pathways include non-homologous end-joining (NHEJ) and homologous recombination (HR). NHEJ is a rapid, high-capacity pathway that joins two DNA ends using ligase IV/XRCC4 (X-Ray Repair Cross Complementing 4) complex that recognizes DSBs. NHEJ can, however, accommodate very limited base pairing between the two processed DNA ends, thereby potentially forming repair joints with up to four base pairs of ‘microhomology.’ By contrast, HR requires extensive sequence homology between the broken DNA and a donor DNA molecule. The end resection regulated by EXO1 (exonuclease 1) at DSBs and the DNA synthesis using intact homologous DNA sequence as templates are the key steps in the HR repair process. The Fanconi Anemia (FA) pathway is closely linked to HR repair through its functional interaction with BRCA1/2. FA-group D2 (FANCD2) protein promotes HR repair and prevents DNA DSB formation and chromosomal aberrations in DNA damaged cells. Most DNA repair pathways are complex, involving many proteins working in discrete consecutive steps. Therefore, the efficiency of DNA repair requires transcription factors controlling and maintaining the expression of DNA repair genes. DNA DSB repair is a critical prerequisite for cancer cell survival; it may also provide therapeutic opportunities.

There is a need for more effective therapies for ovarian cancer. As described herein, Compound B has shown enhanced anti-cancer activity when combined with chemotherapeutic drugs such as cisplatin, carboplatin, and paclitaxel, and little or no hematopoietic toxicity in pre-clinical studies. Thus, Compound B is a promising candidate for combination therapies for more effective treatment of ovarian and breast cancers.

Provided herein are methods of treating ovarian cancer in a patient in need thereof, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

Also provided are methods of increasing or enhancing apoptosis of ovarian cancer cells in a patient having ovarian cancer, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

Also provided are methods of treating platinum-resistant ovarian cancer in a patient in need thereof, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

Also provided are methods of enhancing sensitivity of ovarian cancer cells to a chemotherapeutic drug in a patient having ovarian cancer, comprising contacting the cells with therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

Also provided are methods of increasing or enhancing carboplatin-induced DNA damage in a patient having ovarian cancer, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

Also provided are methods of increasing or enhancing sensitivity to combinations of carboplatin and paclitaxel in a patient having ovarian cancer, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

Also provided are methods of prolonging survival in a cancer patient in need thereof comprising administering to the patient in need thereof therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

Also provided are methods of suppressing tumor growth in a cancer patient in need thereof, comprising administering to the patient in need thereof therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

These and other embodiments disclosed herein are described in detail below.

### BRIEF DESCRIPTION OF THE SEQUENCES

SEQ ID NO:1—FANCD2D forward primer.

SEQ ID NO:2—FANCD2D reverse primer.

SEQ ID NO:3—EXD2 forward primer.

SEQ ID NO:4—EXD2 reverse primer.

SEQ ID NO:5—XRCC forward primer.

SEQ ID NO:6—XRCC reverse primer.

SEQ ID NO:7—Sequence of exon 2 of SIK2 for CRISPR/Cas9 knockout in OVCAR8 and SKOv3 cell lines.

SEQ ID NO:8—Sequence of exon 4 of SIK2 for CRISPR/Cas9 knockout in OVCAR8 and SKOv3 cell lines.

### BRIEF DESCRIPTION OF THE DRAWINGS

FIG. 1—Shows that SIK2 inhibitors enhance olaparib sensitivity in ovarian cancer and breast cancer cells. (A) Dose-response curves for Compound A or Compound B (blue), olaparib (green) or Compound A or Compound B combined with olaparib (red) for 96 hrs in 12 cancer cell lines and 3 non-malignant cell lines. The IC5os of inhibitors and concentration ratio of SIK2 inhibitors to olaparib used in each cell line are listed in Table 2. The statistical significance between olaparib alone and SIK2 inhibitor combined with olaparib was calculated with two-way ANOVA multiple comparisons. ***p<0.001, ****p<0.0001, ^{ns}p>0.05 (red stars indicate SIK2 inhibitor+olaparib enhancing the effect of olaparib alone; blue stars indicate SIK2 inhibitor+olaparib inhibiting olaparib's effect. A combination index (CI) at ED 90 was calculated using CalcuSyn software. Representative experiments were from two independent experiments with four technical repeats per experiment. (B) Dose-response curves of olaparib in paired cancer cell lines with or without knockout of SIK2 (top) and with or without stable transfection of SIK2 (bottom). The median inhibitory concertation (IC50) of olaparib was calculated using GraphPad Prism 8. Representative experiments are from two independent experiments with four technical repeats per experiment. Western analysis confirmed either SIK2 knock out (top) or overexpression (bottom). (C) Representative images of clonogenic assays (top) and quantification of colonies (bottom) in four cancer cell lines are presented. SKOv3, OVCAR8, HCC5032, and MDA-MB-231 cells were treated with olaparib, Compound A, Compound B, or olaparib+Compound A or Compound B at concentrations indicated in FIG. 2A for 10-22 days. The columns indicate the mean of colonies and the bars indicate the S.D. (**p<0.01, ***p<0.001, ****p<0.0001). The data were obtained from three independent experiments.

FIG. 2—Shows that SIK2 inhibitors enhance rucaparib, olaparib, niraparib and talazoparib sensitivity in ovarian cancer. (A) Representative images of clonogenic assay in four cancer cell lines are presented (left). SKOv3, OVCAR8, HCC5032, and MDA-MB-231 cells were treated with olaparib, Compound A, Compound B alone, or olaparib plus Compound A or Compound B at concentrations indicated for 10-22 days (right). (B) Dose-response curves of Compound A/Compound B (blue), PARP inhibitors (rucaparib, olaparib, niraparib, or talazoparib) (green) or Compound A/Compound B combined with PARP inhibitor (red) for 96 hrs in OVCAR8 and SKOv3 ovarian cancer cells. Combination index (CI) was calculated using CalcuSyn software. Representative experiments were from two independent experiment and 4 technical repeats per experiment.

FIG. 3—Shows the effect of Compound A, Compound B, and olaparib on PARP1 enzyme activity and trapping. (A) PARP1Trapping in OVCAR8 and MDA-MB-231 cells. Cells were treated with Compound A, Compound B, olaparib alone, or olaparib+Compound A or Compound B for 72 hrs. The concentrations of Compound A, Compound B, and olaparib are 4 μM, 4 μM, and 6 μM, respectively. Western blot analysis of chromatin-bound fractions of PARP1. (B) Western blot analysis of PARP1 protein expression. (C) The dose-response effect of olaparib and SIK2 inhibitor on PARP1 enzyme activity. OVCAR8 and MDA-MB-231 cells were treated with SIK2 inhibitors for 26 hrs as indicated. The columns indicate the mean of activity and the bars indicate the S.D. (^{ns}p>0.05, **p<0.01, ***p<0.001, ****p<0.0001). Representative experiments were from three independent experiments and 4 technical repeats per experiment.

FIG. 4—Shows the combined effect of SIK2 inhibitor and olaparib on PARP-1 enzyme activity and DNA DSB repair pathways. (A) Dose-response curves for olaparib (top) and combined effect of SIK2 inhibitors with olaparib on PARP-1 enzyme activity (bottom). OVCAR8 and MDA-MB-231 cells were treated with SIK2 inhibitors, olaparib alone, or the combination for 26 hrs. The concentrations of Compound A, Compound B, and olaparib are 6 μM, 4 μM, and 0.05 μM, respectively (also see FIG. 3B-C). The columns indicate the mean of activity and the bars indicate the S.D. (*p<0.05, **p<0.01, ***p<0.001, ****p<0.0001). Representative data are from three independent experiments with 4 technical repeats per experiment. (B) Dose-response curves of Compound A, Compound B, and olaparib in DT40 PARP-1−/− cells with and without knock-in of human PARP-1 (hPARP) (top and bottom left panels). The IC50 indicated on the curves was calculated using GraphPad Prism 8. The expression of exogenous hPARP in DT40 PARP-1−/− was measured by western blotting (bottom left panel). Representative data were from two independent experiments with 4 technical repeats per experiment. (C) The heatmap presentation of unsupervised hierarchical clustering of gene expression. The heatmap includes 3587 transcripts (up or down-regulated by ≥2-fold) treated with Compound A, Compound B, olaparib, Compound A+olaparib and Compound B+olaparib. The heatmap illustrates changes that are color coded with red corresponding to up-regulation and green to down-regulation. (D) The Venn representation. Venn diagram analysis represented the number of genes (up or down-regulated by ≥2-fold) were overlapped by the treatment of Compound A+olaparib (yellow) or Compound B+olaparib (green). (E) Go analysis of 1380 differentially expressed genes shared by Compound A+olaparib or Compound B+olaparib treatments. The bar plot shows the log10 P value of the biological process GO terms obtained with differentially expressed genes at p<0.01. Red highlights indicate biological processes involved in DNA damage and repair.

FIG. 5—Shows that Compound A and Compound B enhances olaparib-induced DNA DSBs and apoptosis. (A) The Heatmap representation unsupervised hierarchical clustering of differentially expressed genes associated with DNA repair. The heatmap contains changes that are color coded with red corresponding to up-regulation and green to down-regulation. (B) Analysis of DNA Repair and apoptosis genes. BRCA2, EXO1, FANCD2, LIG4, XRCC4, BAX, BCL2, CASP7, and TRADD were analyzed using RT-PCR in OVCAR8 ovarian and MDA-MB-231 breast cancer cells. Cells were treated with Compound A, Compound B, olaparib alone, or olaparib+Compound A or Compound B for 72 hrs. The concentrations of Compound A, Compound B, and olaparib are 4 μM (2 times), 4 μM (3 times), and 15 μM (2 times), respectively. The columns indicate the mean of RNA expression and the bars indicate the S.D. (*p<0.05, **p<0.01, ***p<0.001, ****p<0.0001). Representative data are from two independent experiments with 3 technical repeats per experiment. (C) Quantification of DNA damage (γ-H2AX). Endogenous γ-H2AX was stained with anti-γ-H2AX antibody in the cells treated with single agent or combined for 8 hrs as indicated. The concentrations of Compound A, Compound B, and olaparib were 1 μM, 4 μM, and 2 μM, respectively. Red indicates γ-H2AX and Blue-DAPI indicates nuclear stain. Representative images are presented (right). Bar 20 μm. Red γ-H2AX dots were quantified with OLYMPUS CellSens Dimension software. The middle solid lines indicate the mean of fluorescent dots. The top and bottom solid lines indicate the S.D. (***p<0.001, ****p<0.0001). (^{ns}p>0.05, **p<0.01, ***p<0.001, ****p<0.0001) (left). Experiments were from three independent experiments with a total of 100-200 cells per treatment. Bar 20 μm. (D) Detection of apoptosis using Annexin V/Propidium iodide (PI) staining. SKOv3 cells were treated with Compound A (8 μM), Compound B (5 μM), olaparib (25 μM) alone or combined for 6 days as indicated. HCC5032 cells were treated with Compound A (1 μM), Compound A (3 μM) or olaparib (3 μM) alone or combined for 5 days. OVCAR8 and MDA-MB231 were treated with treated with Compound A (6 μM), Compound B (6 μM) or olaparib (5 μM) alone or combined for 5 days. The columns indicate the mean of Annexin V positive cells and the bars indicate the S.D. (*p<0.05, **p<0.01, ***p<0.001, ****p<0.0001). Representative data are from three independent experiments with 3 technical repeats per experiment.

FIG. 6—Shows that Compound A and Compound B enhance olaparib-induced DNA double-strain breaks and apoptosis. (A) The heatmap of unsupervised hierarchical clustering of differently expressed genes associated with apoptosis. The map contains changes that are color coded with red corresponding to up-regulation and green to down-regulation. (B) Analysis of DNA repair and apoptosis gene. BRCA2, EXO1, FANCD2, LIG4, XRCC4, BAX, BCL2, CASP7, and TRADD were analyzed using RT-qPCR in SKOv3 and OVCAR8 ovarian cancer cells. Cells were treated with Compound A, Compound B, olaparib alone, or olaparib+Compound A or Compound B for 72 hrs. The concentrations of Compound A, Compound B, and olaparib are 4 μM (2 times), 4 μM (3 times), and 15 μM (2 times), respectively. The columns indicate the mean of RNA expression and the bars indicate the S.D. (*p<0.05, **p<0.01, ***p<0.001, ****p<0.0001). Representative experiments were from two independent experiment and 3 technical repeats per experiments. (C) Quantification of DNA damage using comet assay. Cells were plated and treated with Compound A, Compound B, or olaparib on the comet slides for total 48 hrs and with and without olaparib for 16 hrs (16 hrs before harvest). 1 μM of Compound A was applied to HCC5032, OVCAR8, and SKOv3 and 0.5 μM to MDA-MB-231 cells. 4 μM of Compound B and 5 μM of olaparib are applied to all 4 cell lines tested. Slides were then stained with Vista Green DNA dye and viewed using Olympus fluorescence microscope with FITC filter. Olive Tail Moment was measured using CaspLab1.2.3β2 software. The columns indicate the mean of tail moments and the bars indicate the S.D. (*p<0.05, **p<0.01, ***p<0.001, ****p<0.0001). Representative experiments were from three independent experiments with a minimum of 50 cells.

FIG. 7—Shows that Compound A and Compound B decrease phosphorylation of HDAC4/5/7 and promoter activity of MEF2 transcription factors. (A) Phosphorylation level of HDAC4/5/7. Twenty-one ovarian and one triple-negative breast cancer cell lines were treated with Compound A (4 μM) (top panel) or Compound B (4 μM) (bottom panel) for 24 hrs. Western blots were probed with specific antibodies as indicated. (B) Detection of HDAC5localization with or without SIK2 inhibitors. OVCAR8 and MDA-MB-231 cells were plated on 2-well chamber slides. After overnight incubation, cells were treated with Compound A (3 μM) or Compound B (5 μM) for 24 hrs. Cells were stained with anti-HDAC5 and imaged with fluorescence microscopy for HDAC5 (green) and DAPI nuclear stains (blue). The fluorescent intensity of nuclear HDAC5 was quantified using ImageJ (FIG. 8). The bar represents 20 μm. Data were from three independent experiments with a total of 100-200 cells per group. (C) Quantification of MEF2 promoter activity. Cells were plated and incubated overnight. Cells were then transfected with a mixture of a MEF2-responsive luciferase construct and a constitutively expressing Renilla luciferase construct (40:1) for 24 hrs. Cells were re-plated into 96 well plates and then treated with Compound A (4 μM) and Compound B (4 μM) for different intervals or with different doses of Compound A and Compound B for 24 hrs as indicated. Cells were lysed for dual luciferase assays. The relative luciferase activity of MEF2 was calculated by normalizing to Renilla luciferase activity. The columns indicate the mean of MEF2 luciferase activity and the bars indicate the S.D. (^{ns}p>0.05, *p<0.05, **p<0.01, ***p<0.001, ****p<0.0001). Representative data were from two independent experiments with 3 technical repeats per experiment. (D) Quantification of MEF2 promoter activity with and without knockdown of HDAC4 and HDAC5. Cells were transfected with targeting or control siRNA for 24 hrs prior to transfection of a mixture of a MEF2-responsive luciferase construct and Renilla luciferase construct. Cells were re-plated into 96 well plates and then treated with Compound A (4 μM) or Compound B (4 μM) for 24 hrs. Luciferase activity was measured and analyzed as described in (C) (top panel). HDAC4 and HDAC5 siRNA knockdown efficiency was measured by western blot analysis (bottom panel). Representative data are from two independent experiments.

FIG. 8—Shows that Compound A and Compound B decrease phosphorylation of HDAC4/5/7 and promoter activity of MEF2 transcription factors. (A) Detection of HDAC5 localization with and without SIK2 inhibitors using immunofluorescence staining. Nuclear fluorescent intensity was measured by ImageJ (related to FIG. 7B). Experiments were from three independent experiments with total 100-200 cells per group. The middle solid lines indicate the mean of fluorescent intensity. To top and bottom solid lines indicate the S.D. (***p<0.001, ****p<0.0001). (B) Detection of HDAC5 localization with and without SIK2 inhibitors using cell fractionation. OVCAR8 and MDA-MB-231 cells were treated with Compound A (6 μM) or Compound B (5 μM) for 26 hrs. Total cell lyses were collected for cell fractionation and cytoplasmic extracts and nuclear extracts were subjected to western analysis using the antibodies indicated. (D and L indicate dark and light exposure, respectively). (C) Quantification of MEF2 promoter activity (related to FIG. 7C). Cells were plated and after overnight incubation, then transfected with a mixture of a MEF2-responsive luciferase construct and a constitutively expressing Renilla luciferase construct (40:1) (QIAGEN) for 24 hrs. Cells were re-plated into 96 well plate and then treated with olaparib (4 μM) for different time intervals or with different doses of olaparib for 24 hrs as indicated. Cells were lysed for dual luciferase assay. The relative luciferase activity of MEF2 was calculated by normalizing to Renilla luciferase activity. The columns indicate the mean of MEF2 luciferase activity and the bars indicate the S.D. (^{ns}p>0.05, *p<0.05). Representative experiments were from two independent experiments and 3 technical repeats per experiment. (D) Quantification of MEF2 promoter activity (related to FIG. 7D). Cells were treated with TMP195 for 24 hrs prior to transfection of a mixture of a MEF2-responsive luciferase construct and Renilla luciferase construct. Cells were re-plated into 96 well plate and then treated with Compound A (4 μM) and Compound B (4 μM) for 24 hrs. Measurement of luciferase activity is performed, quantified, and analyzed as described in (C) (top panel). The bars indicate the S.D. (^{ns}p>0.05, *p<0.05, ****p<0.0001). Representative experiments were from two independent experiments and 3 technical repeats per experiment. (E) Working model. SIK2 inhibitor inhibits class IIa HDAC/MEF2D-mediated downregulation of genes that are associated with DNA repair.

FIG. 9—Shows that SIK2 inhibition alters MEF2D transcription factor-mediated downstream signaling. (A) Alterations affecting MEF2 family genes in ovarian and breast cancer by TCGA analysis. Alterations of MEF2D are found in 12% of ovarian cancer samples (TCGA, 316 samples, Nature 2011) and 26% of breast cancer samples (Metabric, 2509 samples, Nature 2012 & Nat Commun 2016), respectively, and the large majority of alterations were amplifications and mRNA upregulations. Data and plots were obtained using cBioPortal (21, 58, 59). (B) MEF2D consensus DNA motifs. The MEF2 motif is enriched in MEF2D-binding sites in SKOv3 cells. (C) GO analysis of MEF2D-bound genes. (D) Chip sequence of anti-MEF2D at the FANCD2 locus in SKOv3 cells treated with and without Compound A. The dotted line indicates the comparison of chromatin accessibility of the FANCD2 gene between control and Compound A treatment. (E) Chip and RT-qPCR analysis of FANCD2, EXO1, and XRCC4 genes. OVCAR8 and MDA-MB-231 cells were treated with and without Compound A (6 μM) or Compound B (4 μM) for 48-50 hrs and then harvested subjecting to Chip with normal IgG, MEF2D, Pol-II, H3K27Ac, or H3KMe1 antibody as indicated. Chip pull-down samples were analyzed by RT-qPCR. The columns indicate the mean of relative fold changes (Fold change=2-DDCt, Chip signal relative to the IgG background signal) and the bars indicate the S.D. (*p<0.05, **p<0.01, ***p<0.001, ****p<0.0001). Representative data are from two independent experiments and 3 technical repeats per experiment.

FIG. 10—Shows that SIK2 inhibition alters MEF2D transcription factor-mediated downstream signaling (related to FIG. 9). (A) MEF2D-binding sites in human ovarian cancer cells. (B) Chip analysis of FANCD2, EXO1, and XRCC4 genes. SKOv3, SKOv3- and OVCA8-SIK2 knockout cells were treated with and without Compound A (6 μM) or Compound B (4 μM) for 48-50 hrs and then harvested subjecting to Chip with normal IgG, MEF2D, Pol-II, H3K27Ac, or H3KMe1 antibody as indicated. Chip pull-down samples were analyzed by RT-qPCR using as indicated. The columns indicate the mean of relative fold changes (Fold change=2-DDCt, Chip signal relative to the IgG background signal) and the bars indicate the S.D. (*p<0.05, **p<0.01, ***p<0.001, ****p<0.0001). Representative experiments were from two independent experiments and 3 technical repeats per experiment.

FIG. 11—Shows clinical data analysis by log-rank test (gepia.cancer-pku.cn/). Kaplan Meier survival curves of FANCD2, EXO1, and XRCC1 in ovarian and breast cancer.

FIG. 12—Shows that overexpression of MEF2D is sufficient to block SIK2 inhibition-induced downregulation of FANCD2, EXO1, and XRCC4, DNA damage and growth inhibition. (A) Forced expression of MEFD2. DOX-Inducible MEF2D expression OVCAR8 and MDA-MB-231 cells were treated with Compound A (1 μM), Compound B (4 μM), and olaparib (2 μM) in present and absent of DOX (1 μg/ml) for 8 hrs. DOX was added to culture medium 48 hrs prior to inhibitor treatments. Red indicates γ-H2AX and Blue-DAPI indicates nuclear stains. Reprehensive images were presented (Left). Bar 20 μm. Red γ-H2AX dots were quantified with OLYMPUS CellSens Dimension software. The middle solid lines indicate the mean of fluorescent dots. To top and bottom solid lines indicate the S.D. (^{ns}p>0.05, **p<0.01, ***p<0.001, ****p<0.0001) (right). Bar 20 μm. Representative experiments were from two independent experiments and 3 technical repeats per experiment. (B) Determination of MEF2D expression by western analysis. (C) Determination of cell viability in MEF2D DOX-Inducible OVCAR8 and MDA-MB-231 cells. DOX-Inducible MEF2D sublines of OVCAR8 and MDA-MB-231 were treated with DOX and without DOX for 24 hrs, and then treat with Compound A (2 μM), Compound B (4 μM), and olaparib (4 μM) for 72 hrs. The statistical significance between DOX− and DOX+ was calculated with one-way ANOVA multiple comparisons. ****p<0.0001, ^{ns}p>0.05. Representative data are from three independent experiments with 4 technical repeats per experiment.

FIG. 13—Shows that co-administration of SIK2 inhibitor and olaparib synergistically inhibits xenograft growth. (A) Tumor growth and (B) Tumor weight of ovarian cancer xenografts in female athymic nu/nu mice after treatment with Compound A, Compound B, olaparib, Compound A+olaparib, and Compound B+olaparib. SKOv3 (5×10⁶) or OVCAR8 (3×10⁶) cells were injected subcutaneously (sub-q) or intraperitoneally (ip). After 7-day inoculation, mice (n=8-10) were treated with Compound A, Compound B, olaparib, or combination as indicated by gavage 5 days per week for 4-6 weeks. Tumor growth by tumor volume (A) or tumor weight (B)under different treatments was plotted as mean±S.D. (*p<0.05; **p<0.01). (C) Tumor weight of ovarian cancer cells in female athymic nu/nu mice after treatment with Compound B, olaparib, and Compound B+olaparib. 3.5×10⁶ OC316 tumor cells were injected i.p. On day 7 after inoculation, mice (n=20) were treated with Compound A, Compound B, olaparib, or a combination as indicated by gavage 5 days per week for 5 weeks. Mice with ip tumor growth (n=10 mice per group) were sacrificed and tumors were weighed after completing 5 weeks of treatment. Tumor growth by weight under different treatments was plotted as mean±S.D. (*p<0.05; **p<0.01). Survival (ethical endpoint) of the remaining 10 mice per group was evaluated. Survival curves were generated by GraphPad Prism 6. (^{ns}p>0.05, *p<0.05; **p<0.01). (C) Tumor growth of MDA-MB-231 breast cancer cell in female athymic nu/nu mice. 0.8×10⁶ MDA-MB-231 cells were injected into the fourth mammary fat pads. Tumor-bearing mice were randomized into 4 treatment groups (n=10) after 7-days of tumor growth. Mice were treated with Compound B, olaparib, and Compound B+olaparib for 5 weeks as indicated. Tumor growth was measured and survival (ethical endpoint) was evaluated from the start of treatment until tumors reached 1000 mm³. Survival curves were generated by GraphPad Prism 6. (^{ns}p>0.05, *p<0.05; **p<0.01). (E) Representative images of IHC with indicated antibodies from OVCAR8 and MDA-MB-231 mouse tumor tissues. Scale bar, 50 μM. Positive cells per one hundred cancer cells were counted and analyzed using GraphPad Prism 8 (^{ns}p>0.05, **p<0.01, ***p<0.001, ****p<0.0001). #1 indicates mouse #1 and #2 indicates mouse #2.

FIG. 14—Shows that co-administration of SIK2 inhibitor and olaparib is synergistic in vivo (related to FIG. 13). Both mice body weights and ascites volume of OVCAR8 (A) and OC316 (B) intraperitoneal models were evaluated after end of experiments. The middle solid lines indicate the mean of ascites volume (left) or body weight (right). (^{ns}p>0.05, **p<0.01, ***p<0.001).

FIG. 15—Shows SIK2 expression in the breast cancer tissue and cell lines. (A) Immunohistochemistry staining of TMA and analysis of SIK2 expression in the bar graph and (B) SIK2 expression in breast cancer cell lines by western blot analysis.

FIG. 16—Shows that Compound B enhances paclitaxel sensitivity. (A) Compound B inhibits organoid growth, inducing cell death. (B) SIK2 expression is inversely correlated with SIK2 expression, p=0.0277. (C) Compound B enhanced paclitaxel sensitivity, inhibiting growth of MDA-MB-231 xenografts (top) and prolonging survival of mice bearing MDA-MB-231 xenografts (bottom), *p<0.05 and **p<0.01, and (D) Compound B and paclitaxel showed synergistic cytotoxicity judging by CI value less than 1.

FIG. 17—Demonstrates that Compound B synergistically enhances carboplatin-induced inhibition of ovarian cancer cell short-term and clonogenic growth in cell culture. (A) Sensitivity to Compound B. A2780, ES2, IGROV1, MDA2774, OC316, OVCAR3, OVCAR8, and SKOv3 ovarian cancer cell lines were plated at a density of 2000 cells/well in 96-well plates, then treated with different concentrations of Compound B for 96 h. Cell viability was measured with a bioluminescence assay as described in the Examples, and IC50 values were calculated. (B) Sensitivity to carboplatin. Eight ovarian cancer cell lines were treated as above with different concentrations of carboplatin as indicated. (C) Effect of a single concentration of Compound B on the carboplatin dose response curve. Eight ovarian cancer cell lines were treated with different concentrations of carboplatin as indicated with or without a single concentration of Compound B (A2780 0.75 μM, ES2 1.25 μM, IGROV1 1.25 μM, MD2774 1.15 μM, OC316 0.75 μM, OVCAR3 0.75 μM, OVCAR8 1 μM, and SKOv3 1 μM). IC50s of carboplatin with or without Compound B were calculated by GraphPad Prism 8 (**p<0.01 by student t test). (D) Synergistic interaction of carboplatin and Compound B. IGROV1, OC316, OVCAR8, and SKOv3 were treated concomitantly with a serial dilution of Compound B and carboplatin at a fixed ratio indicated in the figure. The drug concentration ratio is indicated in each plot. The combination index at 50% growth inhibition was calculated using CalcuSyn software. (E) Effect of SIK2 knockout on the carboplatin dose response curve. Cells were treated with different concentrations of carboplatin as indicated. IC50 values for (A-C, E) were calculated by GraphPad Prism 8. (F) Compound B enhances carboplatin-induced inhibition of clonogenic growth. Four hundred OVCAR8 or SKOv3 ovarian cancer cells were seeded in 6-well plates in culture medium for 24 h. Cells were then treated with diluent, Compound B (ES2 2.2 μM, OC316 2.5 μM, OVCAR8 2.3 μM, SKOv3 3.5 μM, and MDA2774 2.5 μM), carboplatin (ES2 3.3 μM, OC316 3.0 μM, OVCAR8 4.0 μM, SKOv3 2.0 μM, and MDA2774 3.0 μM) or both in triplicate for another 12-14 days. The graphs indicate the mean colony formation numbers with standard deviations. Statistical significance is indicated by *p<0.05, **p<0.01, ***p<0.001, and ****p<0.0001 by one-way ANOVA analysis.

FIG. 18—Shows that Compound B enhances carboplatin-induced inhibition of clonogenic growth. 400 OVCAR8 or SKOv3 ovarian cancer cells were seeded in 6-well plates in normal culture medium for 24 hrs. Cells were then treated with diluent, Compound B (ES2 2.2 μM, OC316 2.5 μM, OVCAR8 2.3 μM, SKOv3 3.5 μM, and MDA2774 2.5 μM), carboplatin (ES2 3.3 μM, OC316 3.0 μM, OVCAR8 4.0 μM, SKOv3 2.0 μM, and MDA2774 3.0 μM), or both in triplicate for another 12-14 days.

FIG. 19—Shows that inhibition of SIK2 activity with Compound B or knockout of SIK2 protein enhances carboplatin-induced apoptosis. (A) Effect of Compound B on carboplatin-induced apoptosis. OC316, OVCAR8, and SKOv3 cell lines were plated at a density of 8000 cells/well in 12-well plate in triplicate, and then treated with Compound B (OC316 3 μM, OVCAR8 5 μM, and SKOv3 4.5 μM) and/or carboplatin (OC316 15 μM, OVCAR8 70 μM, and SKOv3 60 μM) for 72 h. Cells were dislodged and stained with Annexin V antibody and PI dye for flow cytometry. Representative images are shown on the left and the analysis of apoptotic population under different treatment conditions are on the right. (B) Effect of SIK2 knockout on carboplatin-induced apoptosis. SIK2 knockout (KO) and control cell lines were treated as in (A) and analyzed for apoptosis. The bars indicate the mean percentage of apoptotic cells with standard deviations. Statistical significance is indicated by *p<0.05, **p<0.01, ***p<0.001, and ****p<0.0001. ns: not significant by one-way ANOVA analysis.

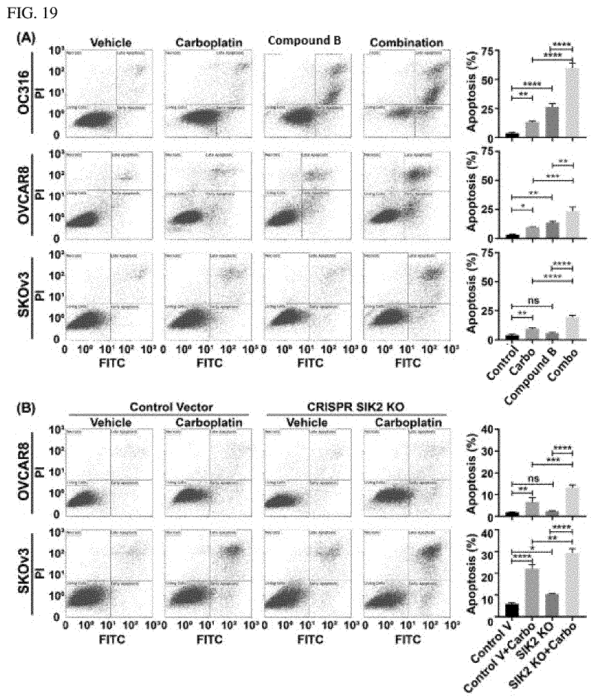

FIG. 20—Shows that treatment with Compound B enhances the carboplatin-induced decrease in survivin expression. OC316, OVCAR8, and OVCAR8 SIK2 KO ovarian cancer cells were treated with diluent, Compound B (OC316 3 μM and OVCAR8 5.0 μM), carboplatin (OC316 15 μM and OVCAR8 60 μM) and the combination for 48 h. Cell lysates were collected and survivin expression was measured by western blot analysis. The experiments were performed three times individually. Densitometry values were determined by Image J shareware (NIH) and normalized to the GAPDH loading control. The values relative to the untreated group were plotted at the bottom. Different treatments were compared by one-way ANOVA analysis. *p<0.05 and **p<0.01 compared to untreated control group; #p<0.05, ##p<0.01, and ###p<0.001 compared to the combination treatment of Compound B and carboplatin.

FIG. 21—Shows Compound B enhances carboplatin-induced DNA damage. (A) OC316, OVCAR8, and SKOv3 ovarian cancer cells were treated with diluent, Compound B (OC316 3 μM, OVCAR8 3.0 μM, and SKOv3 3.5 μM), carboplatin (OC316 15 μM, OVCAR8 35 μM, and SKOv3 35 μM) or the combination for 8 h and stained for γ-H2AX in green and for DNA with DAPI in blue. Each plot depicts the mean number of punctae (the bars indicate the standard deviation). (B) Cells were treated as described in (A) for 24 h. Then cells were dislodged, immobilized in agarose gel onto glass slide, and lysed. DNA was electrophoresed in alkaline buffer and stained by Vista Green. Olive tail moment (OTL) was measured as described in the Examples. Each plot depicts the mean of OTL (the bars indicate the standard deviation). Statistical significance by one-way ANOVA is indicated by *p<0.05, **p<0.01, ***p<0.001, and ****p<0.0001.

FIG. 22—Shows Treatment with Compound B enhances carboplatin toxicity in cisplatin-sensitive and cisplatin-resistant sub-lines. (A) A cisplatin-resistant ovarian cancer cell sub-line is also resistant to carboplatin. Cisplatin-resistant A2780-CP20 and cisplatin-sensitive A27801 PAR sub-lines were plated at a density of 2000/well in 96-well plates, then treated with different concentrations of carboplatin for 96 h as indicated. Cell viability was measured with a bioluminescence assay and the IC50 was calculated. (B) Growth of both cisplatin-resistant and cisplatin-sensitive sub-lines are inhibited by Compound B as indicated. Cells were similarly cultured and treated for 96 h with different concentrations of Compound B, before measuring cell viability and calculating IC50. (C, D). The interaction of Compound B and carboplatin in cisplatin-sensitive (C) and cisplatin-resistant cell lines was evaluated by the combination index at 50% growth inhibition using CalcuSyn software.

FIG. 23—Shows Compound B enhances the activity of carboplatin in human ovarian cancer cell xenografts. (A) Design of xenograft experiments (n=10/group); (B) the combination of Compound B and carboplatin inhibits tumor growth in an OVCAR8 i.p. model. After treatment as indicated in (A) for three weeks, mice were weighed, and intraperitoneal nodules were excised and weighed. (C) Design of xenograft experiments (n=10/groups); (D) The combination of Compound B and primary chemotherapeutic drugs carboplatin and paclitaxel inhibits tumor growth in an SKOv3 subcutaneous xenograft model. Mice were treated with single, double, or triple agents for 6 weeks. Tumor was measured once a week until the tumor burden in control group reached maximum allowance. The graphs indicate the mean±standard deviation. Statistical significance is indicated by *p<0.05, **p<0.01, and ***p<0.0001.

FIG. 24—Shows dose response curves for Compound B and its isoforms against SIK1 (left), SIK2 (middle), and SIK3 (right).

FIG. 25—Shows a cell viability assay for paclitaxel and Compound B.

FIG. 26—Shows a cell viability assay for paclitaxel (left), cisplatin (middle), and Compound B (right).

FIG. 27—Shows a cell viability assay for Compound B+paclitaxel (Combination 2, left) and Compound B+cisplatin (Combination 2, right).

FIG. 28—Shows the combination effect of Compound B and paclitaxel on SK-OV-3 cell cycle. Left: positive control (untreated cells); right: Compound B.

FIG. 29—Shows the combination effect of 30 μM Compound B and 3 μM paclitaxel on SK-OV-3 cell cycle.

FIG. 30—Shows the effect of Compound B and paclitaxel on SIK2 mRNA expression in SK-OV-3 xenograft tumor samples. A (left, Compound B); B (middle, paclitaxel); C (right, xenograft study).

FIG. 31—Shows effects of Compound B alone and in combination on SK-o-V3 xenografts.

FIG. 32—Shows effects of Compound B alone and in combination with cisplatin on SK-o-V3 xenografts.

FIG. 33—Shows effects of Compound B alone and in combination with paclitaxel on SK-o-V3 xenografts.

FIG. 34—Shows effects of Compound B p.o. and i.p. on SK-o-V3 xenografts.

FIG. 35—Shows the antitumor effect of Compound B alone and in combination with paclitaxel.

FIG. 36—Shows the antitumor effect of Paclitaxel alone and in combination with Compound B.

FIG. 37—Shows the antitumor effect of Compound B alone and in combination with paclitaxel.

FIG. 38—Shows the antitumor effect of Compound B alone and in combination with cisplatin.

### DETAILED DESCRIPTION

### Overview

Provided herein are methods of treating ovarian cancer in a patient in need thereof, comprising administering to the patient therapeutically effective amounts of:
a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of increasing or enhancing apoptosis of ovarian cancer cells in a patient having ovarian cancer, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of treating platinum-resistant ovarian cancer in a patient in need thereof, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of enhancing sensitivity of ovarian cancer cells to a chemotherapeutic drug in a patient having ovarian cancer, comprising contacting the cells with therapeutically effective amounts of: a SIK2 inhibitor and carboplatin; or a SIK2 inhibitor and a combination of paclitaxel and cisplatin; or
a SIK2 inhibitor and a combination of paclitaxel and carboplatin; or a SIK2 inhibitor and a combination of paclitaxel, cisplatin, and carboplatin. Also provided are methods of increasing or enhancing carboplatin-induced DNA damage in a patient having ovarian cancer, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of increasing or enhancing sensitivity to combinations of carboplatin and paclitaxel in a patient having ovarian cancer, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or
a SIK2 inhibitor and a combination of paclitaxel, cisplatin, and carboplatin. Also provided are methods of prolonging survival in a cancer patient in need thereof comprising administering to the patient in need thereof therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of suppressing tumor growth in a cancer patient in need thereof, comprising administering to the patient in need thereof therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin.

Ovarian cancer is a leading cause of gynecological cancer death. Each year, 230,000 women will be diagnosed with ovarian cancer and 150,000 will die from the disease worldwide. High-grade serous ovarian cancer (HGSOC) accounts for 70-80% of ovarian cancer deaths, and long-term survival has not changed significantly for several decades. Most patients are treated with cytoreductive surgery and combination chemotherapy using carboplatin and paclitaxel. Seventy percent of patients with primary disease experience a clinical response, but <20% of patients can be cured with advanced stage disease.

The present Inventors have sought kinases that regulate the response of ovarian cancer cells to chemotherapeutic drugs, e.g., paclitaxel and carboplatin, and whose inhibition might improve outcomes for women with ovarian cancer. One of the most promising targets to date is salt-induced kinase 2 (SIK2), which is overexpressed in 30% of ovarian cancers, associated with decreased progression-free survival. SIK2 belongs to the AMPK family. It is a serine-threonine kinase that regulates centrosome splitting, facilitates cell-cycle progression, actives PI3 kinase, reprograms glucose and fatty acid metabolism, and phosphorylates class IIa HDACs, thus affecting gene expression.

Novel 1H-(pyrazol-4-yl)-1H-pyrrolo [2,3-b] pyridine inhibitors have been developed, e.g., Compound A and Compound B, which compete for ATP binding to SIK2 protein and inhibit SIK2 kinase activity. Compound A inhibits SIK2 activity with an IC50<1 nM, but does not significantly inhibit the other two SIK family members, SIK1 and SIK3, as well as other AMPK family members. Compound B, however, is susceptible to efflux by the P-glycoprotein (P-gp) transporter. Compound B, a clinical lead compound derived from Compound A by introducing a solvent binding sulfone, showed acceptable profiles in cell-based proliferation assays, ADME and in PK/PD studies and resisted efflux through the P-gp transporter. Thus, for clinical use, Compound B appeared more promising than Compound A.

In previous studies, the Inventors discovered that inhibition of SIK2 with Compound A enhanced the sensitivity of ovarian cancer cells to paclitaxel in cell culture and in xenografts. As the primary target of platinum drugs is DNA, sensitivity or resistance to treatment is affected by the ability of cells to recognize and repair drug-induced DNA damage. The present study, on the other hand, was conducted to evaluate whether Compound B could increase DNA damage and enhance response to chemotherapeutic drugs, such as cisplatin, carboplatin and/or paclitaxel.

### Salt Induced Kinase 2 (SIK2) Inhibitors for Treatment of Cancer

Disclosed herein are Salt Induced Kinase 2 inhibitors (SIK2i), Compound A and Compound B, which decrease DNA double-strand break (DSB) repair functions and are useful for treatment of many cancers, including, but not limited to, ovarian cancer or breast cancer. SIK2 is required for centrosome splitting and PI3K activation and regulates cancer cell proliferation, metastasis, and sensitivity to paclitaxel. As described herein, a SIK2 inhibitor, e.g., Compound A or Compound B, sensitizes ovarian cancer cell lines and xenografts to chemotherapeutic drugs, such as cisplatin, carboplatin, and/or paclitaxel, or combinations of these drugs with other chemotherapeutic drugs known in the art. SIK2i inhibit the enzyme activity of poly (ADP-ribose) polymerase inhibitors (PARPi) and phosphorylation of class-IIa histone deacetylase (HDAC) 4/5/7. Furthermore, SIK2i abolish class-IIa HDAC 4/5/7-associated transcriptional activity of MEF2D, decreasing MEF2D binding to regulatory regions with high-chromatin accessibility in FANCD2, EXO1, and XRCC4 genes, resulting in repression of their functions in the DNA DSB repair pathway. Combinations of SIK2i, such as Compound A or Compound B, and at least one chemotherapeutic drug, such as cisplatin, carboplatin, and/or paclitaxel, or combinations of these drugs with other chemotherapeutic drugs known in the art, provide a novel therapeutic strategy to enhance the sensitivity of ovarian and triple-negative breast cancers to chemotherapeutic drugs and provide a more robust response to cancer treatment in a patient.

Salt induced kinase 2 (SIK2) is an AMP kinase-related protein kinase that is required for ovarian cancer cell proliferation and metastasis. The kinase phosphorylates multiple substrates including cNAP1, triggering centrosome splitting, and the regulatory subunit of PI3K, enhancing the pathway's activity. SIK2 also phosphorylates class-IIa HDACs and controls their nuclear/cytoplasm shuttling, thus influencing the activity and nuclear localization of class-IIa HDACs. SIK2 is overexpressed and correlates with poor prognosis in patients with ovarian cancer, e.g., high-grade serous ovarian carcinoma (HGSOC). As described herein, orally administered low molecular weight drugs (e.g., Compound A or Compound B) were developed that inhibit SIK2 at nM concentrations, inhibit growth of ovarian cancer cell lines with an IC50 of 0.8 to 3.5 μM, and inhibit growth of ovarian cancer xenografts, enhancing sensitivity to chemotherapeutic drugs such as cisplatin, carboplatin, and/or paclitaxel.

Compound A is 3-(3,5-difluoro-2-methoxyphenyl)-5-(1-(piperidin-4-yl)-1H-pyrazol-4-yl)-1H-pyrrolo[2,3-b]pyridine and the structure is as follows:

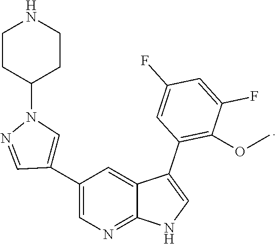 [SMILES: COc1c(F)cc(F)cc1-c1c[nH]c2ncc(-c3cnn(C4CCNCC4)c3)cc12]

Compound B is 3-(3,5-difluoro-2-methoxyphenyl)-5-(1-(1-(methylsulfonyl)piperidin-4-yl)-1H-pyrazol-4-yl)-1H-pyrrolo[2,3-b]pyridine and the structure is as follows:

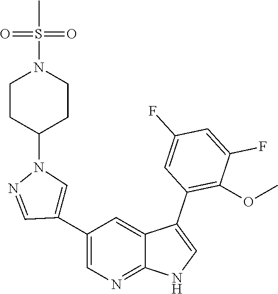 [SMILES: COc1c(F)cc(F)cc1-c1c[nH]c2ncc(-c3cnn(C4CCN(S(C)(=O)=O)CC4)c3)cc12]

Despite promising clinical results for SIK2i or conventional chemotherapeutic drugs known in the art as single agents, high dosage requirements and prevalence of acquired resistance remain challenges to more effective treatment. Combination therapies are of considerable interest for enhancing the efficiency of treatment. The present study discovered that combinations of SIK2 inhibitors and conventional chemotherapeutic drugs, such as cisplatin, carboplatin, and/or paclitaxel, or combinations of these drugs with other chemotherapeutic drugs known in the art, provide an increased or enhanced anticancer response and increased killing of cancer cells in ovarian and triple-negative breast cancer cell lines and xenografts.

### Chemotherapeutic Drugs for Treatment of Ovarian Cancer

Carboplatin and paclitaxel constitute first-line treatment for ovarian cancer, producing tumor shrinkage in 70% of patients, but curing less than 20% with advanced stage disease. Previous studies have shown that treatment with Compound B, a small molecule inhibitor of the enzyme salt-induced kinase 2 described herein, can improve the response to conventional chemotherapeutic drugs, such as cisplatin, carboplatin, and/or paclitaxel, or combinations of these drugs with other chemotherapeutic drugs known in the art, in human ovarian cancer cells grown in culture and in immunocompromised mice. Here, the present Inventors have found that Compound B also increases carboplatin's ability to kill ovarian cancer cells grown in culture and in immunocompromised mice, causing additional DNA damage and decreasing levels of survivin, a protein that protects cancer cells from programmed cell death. These studies encourage clinical evaluation of Compound B, and a Phase I clinical trial has been initiated to test the drug in ovarian cancer patients.

A number of chemotherapeutic drugs are known in the art for treatment of ovarian cancer and may be used in accordance with the present disclosure. For example, a useful chemotherapeutic drug for treatment of ovarian cancer can include platinum-based chemotherapeutic drugs, including, but not limited to, carboplatin, cisplatin, or oxaliplatin. Other useful chemotherapeutic drugs include taxane chemotherapeutic drugs, including, but not limited to, paclitaxel (Taxol®), docetaxel (Taxotere®), or the like. In some embodiments, useful drugs for treatment of ovarian cancer include poly (ADP-ribose) polymerase (PARP) inhibitors, such as olaparib, rucaparib, niraparib, or others known in the art. In some embodiments, useful drugs for treatment of ovarian cancer include anti-neoplastic drugs, such as liposomal doxorubicin, topotecan and related compounds, etoposide and related compounds, gemcitabine and related compounds, docetaxel, vinorelbine, ifosfamide, 5-fluorouracil with leucovorin, and altretamine (Hexalen).

As would be understood by one of skill in the art, any chemotherapeutic drug capable of treating cancer in a patient, e.g., reducing the size of a tumor or the tumor load in a patient, reducing the number of cancer cells in a patient, increasing apoptosis of cancer cells in a patient, increasing DNA damage in cancer cells, or otherwise contributing to treatment of ovarian cancer in a patient, would be useful in accordance with the present disclosure. For example, a chemotherapeutic drug may include, but is not limited to, albumin bound paclitaxel (nab-paclitaxel, Abraxane®), altretamine (Hexalen®), capecitabine (Xeloda®), cyclophosphamide (Cytoxan®), etoposide (VP-16), gemcitabine (Gemzar®), ifosfamide (Ifex®), irinotecan (CPT-11, Camptosar®), liposomal doxorubicin (Doxil®), melphalan, pemetrexed (Alimta®), topotecan, vinorelbine (Navelbine®), bleomycin, etoposide, bevacizumab and related compounds, anthracyclines, In some embodiments, the at least a first chemotherapeutic drug and/or the at least a second chemotherapeutic drug is selected from the group consisting of carboplatin, cisplatin, oxaliplatin, and paclitaxel, or the like. In some embodiments, particularly useful drugs for use with the present disclosure are platinum-based drugs, e.g., carboplatin, cisplatin, or oxaliplatin, although any platinum-based drug known or available in the art can be used as deemed appropriate by a clinician.

In some embodiments, a SIK2 inhibitor may be administered in combination with one or more chemotherapeutic drugs known in the art for treatment of ovarian cancer. For example, combinations of carboplatin and paclitaxel may be useful with a SIK2 inhibitor as described herein. In some embodiments, combinations of carboplatin, paclitaxel, and at least a third chemotherapeutic drug may be useful for treatment of ovarian cancer. In some embodiments, any number of chemotherapeutic drugs may be used together in combination, or in further combination with a SIK2 inhibitor, as deemed appropriate by a clinician, such as 1, 2, 3, 4, 5, 6, 7, 8, 9, 10 chemotherapeutic drugs, or the like.

In some embodiments, combination therapies known in the art for treatment of ovarian cancer known in the art may be used, such as including, but not limited to, TIP (paclitaxel/Taxol®, ifosfamide, and cisplatin/Platinol®), VeIP (vinblastine, ifosfamide, and cisplatin/Platinol®), VIP (etoposide/VP-16, ifosfamide, and cisplatin/Platinol®), and VAC (vincristine, dactinomycin, and cyclophosphamide). Additional treatments or chemotherapeutic drugs are described herein and are intended to be encompassed within the scope of the present disclosure.

Administration of one or more chemotherapeutic drugs in combination with a SIK2 inhibitor as described herein may be by any route appropriate for the drug given, such as intravenous, intraperitoneal, intramuscular, oral, or the like. Treatment may be administered for a specified period of time, e.g., 1, 2, 3, 4, 5, 6, 7, 8, 9, 10 days, or the like; or for 1, 2, 3, 4, 5, 6, 7, 8, 9, 10, weeks, or the like; or for 1, 2, 3, 4, 5 6 7 8 9, 10, 11, 12, 13, 14 15, 16, 17, 18, 19, 20, 21, 22, 23, 24 months or the like. Any length of time or any number of administrations or treatments may be used or given as deemed appropriate by a clinician.

### Class-IIa Histone Deacetylases (HDACs)

Class-IIa histone deacetylases (HDACs) are involved in the regulation of multiple cellular responses. They generally act at the apex of specific genetic programs, by influencing the landscape of genes expressed in a specific context. Class-IIa HDACs do not bind directly to DNA, but rather interact with a selected number of transcription factors, such as Myocyte Enhancer Factor-2 (MEF2), that are recruited to specific genomic regions in a sequence-dependent manner. MEF2 is a MADS box transcription factor originally discovered as a regulator of cardiogenesis and myogenesis. MEF2 influences the expression of numerous genes, individually and cooperatively with other transcription factors. MEF2 can also operate as a transcriptional repressor when complexed with class-IIa HDACs. However, the link between the repressor function of MEF2-class-IIa HDAC axis and expression of DNA repair genes in cancers is not well established.

### Methods of Treatment for Cancer

Provided herein are methods of treating ovarian cancer in a patient in need thereof, comprising administering to the patient therapeutically effective amounts of:
a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of increasing or enhancing apoptosis of ovarian cancer cells in a patient having ovarian cancer, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of treating platinum-resistant ovarian cancer in a patient in need thereof, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of enhancing sensitivity of ovarian cancer cells to a chemotherapeutic drug in a patient having ovarian cancer, comprising contacting the cells with therapeutically effective amounts of: a SIK2 inhibitor and carboplatin; or a SIK2 inhibitor and a combination of paclitaxel and cisplatin; or
a SIK2 inhibitor and a combination of paclitaxel and carboplatin; or a SIK2 inhibitor and a combination of paclitaxel, cisplatin, and carboplatin. Also provided are methods of increasing or enhancing carboplatin-induced DNA damage in a patient having ovarian cancer, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of increasing or enhancing sensitivity to combinations of carboplatin and paclitaxel in a patient having ovarian cancer, comprising administering to the patient therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or
a SIK2 inhibitor and a combination of paclitaxel, cisplatin, and carboplatin. Also provided are methods of prolonging survival in a cancer patient in need thereof comprising administering to the patient in need thereof therapeutically effective amounts of: a SIK2 inhibitor and carboplatin;
or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin. Also provided are methods of suppressing tumor growth in a cancer patient in need thereof, comprising administering to the patient in need thereof therapeutically effective amounts of: a SIK2 inhibitor and carboplatin; or a SIK2 inhibitor and a combination of paclitaxel and cisplatin; or a SIK2 inhibitor and a combination of paclitaxel and carboplatin; or a SIK2 inhibitor and a combination of paclitaxel, cisplatin, and carboplatin.

In some embodiments, the methods described herein may be used to treat cancer in a patient as described herein. In some embodiments, the type of cancer to be treated as described herein may be a cancer type that harbors one or more DNA repair deficiencies described herein. In some embodiments, a cancer type with one or more DNA repair deficiencies is sensitive to SIK2 inhibitors and/or sensitive to chemotherapeutic drugs alone or in combination with other chemotherapeutic drugs and/or in combination with SIK2 inhibitors. In some embodiments, the type of cancer to be treated as described herein may include, but is not limited to, ovarian cancer, endometrial cancer, primary peritoneal cancer, fallopian tube cancer, breast cancer, such as triple-negative breast cancer, prostate cancer, pancreatic cancer, and melanoma. In some embodiments, the type of cancer to be treated is ovarian cancer or breast cancer. Certain types of ovarian or breast cancer may be particularly suited for treatment as described herein, such as including, but not limited to, high-grade serous ovarian carcinoma (HGSOC) or triple-negative breast cancer. In some embodiments, the ovarian cancer is primary cancer. In some embodiments, the ovarian cancer is recurrent cancer. In some embodiments, the ovarian cancer is carboplatin-resistant ovarian cancer. In some embodiments, the ovarian cancer is carboplatin-sensitive ovarian cancer. In some embodiments, the patient achieves remission for cancer and the cancer recurs. In some embodiments, the breast cancer is triple-negative breast cancer and has a mutation in BRCA1/2.

In some embodiments, the type of cancer to be treated as described herein is chosen from prostate cancer, pancreatic cancer, glioblastoma, melanoma, small cell lung cancer, non-small cell lung cancer, gastric cancer, fallopian tube cancer, peritoneal cancer, and testicular cancer.

In some embodiments, the type of cancer to be treated is prostate cancer. In some embodiments, the type of cancer to be treated is pancreatic cancer. In some embodiments, the type of cancer to be treated is glioblastoma. In some embodiments, the type of cancer to be treated is melanoma. In some embodiments, the type of cancer to be treated is small cell lung cancer (SCLC). In some embodiments, the type of cancer to be treated is non-small cell lung cancer. In some embodiments, the type of cancer to be treated as described herein is gastric cancer. In some embodiments, the type of cancer to be treated as described herein is fallopian tube cancer. In some embodiments, the type of cancer to be treated as described herein is peritoneal cancer. In some embodiments, the type of cancer to be treated as described herein is testicular cancer.

In some embodiments, a method described herein further comprises at least a second chemotherapeutic drug. In some embodiments, a method described herein further comprises at least a third chemotherapeutic drug.

In some embodiments, a SIK2 inhibitor described herein may be administered to a patient in combination with one or more chemotherapeutic drugs, e.g., a combination comprising a SIK2 inhibitor and carboplatin; or a combination comprising a SIK2 inhibitor and a combination of paclitaxel and cisplatin; or a combination comprising a SIK2 inhibitor and a combination of paclitaxel and carboplatin; or a combination comprising a SIK2 inhibitor and a combination of paclitaxel, cisplatin, and carboplatin.

In some embodiments, a chemotherapeutic drug described herein can be any chemotherapeutic drug disclosed herein, e.g., a drug selected from the group consisting of carboplatin, cisplatin, oxaliplatin, and paclitaxel. In some embodiments, the chemotherapeutic drug may be topotecan and related compounds, etoposide and related compounds, gemcitabine and related compounds, bevacizumab and related agents, anthracyclines, or the like. Any chemotherapeutic drugs disclosed or described herein may be included in a combination for treatment of cancer, such as ovarian or breast cancer.

In some embodiments, the chemotherapeutic drug is selected from the group consisting of carboplatin and paclitaxel. In some embodiments, the chemotherapeutic drug comprises a combination of carboplatin and paclitaxel.

In some embodiments, the SIK2 inhibitor and the combination of carboplatin and paclitaxel results in a 70% clinical response.

In some embodiments, the combination of the SIK2 inhibitor and the at least a first chemotherapeutic drug inhibits growth of ovarian cancer cells.

In some embodiments, the SIK2 inhibitor is Compound A. In some embodiments, the SIK2 inhibitor is Compound B.

In some embodiments, the SIK2 inhibitor is administered orally.

In some embodiments, the SIK2 inhibitor blocks DNA double-strand break (DSB) repair in the cancer cells.

In some embodiments, the SIK2 inhibitor blocks DNA DSB repair by increasing nuclear localization of histone deacetylase (HDAC) 4/5, wherein the increased nuclear localization of HDAC4/5 blocks the activity of transcription factors associated with DNA DSB repair.

In some embodiments, the transcription factor associated with DNA DSB repair is a myocyte enhancer factor-2 (MEF2) protein. In some embodiments, the MEF2 protein is MEF2D.

In some embodiments, the combination of the SIK2 inhibitor and the at least a first chemotherapeutic drug induces increased levels of apoptosis in the cancer cells compared to cancer cells treated with only the SIK2 inhibitor or the at least a first chemotherapeutic drug.

In some embodiments, the increased levels of apoptosis in the cancer cells is the result of an increase in DNA damage and a decrease in the levels of survivin in the cancer cell.

In some embodiments, the combination of the SIK2 inhibitor and the at least a first chemotherapeutic drug enhances sensitivity of the cancer cells to the at least a first chemotherapeutic drug.

In some embodiments, the combination of the SIK2 inhibitor and the at least a first chemotherapeutic drug produces a synergistic growth inhibition of the cancer cells.

In some embodiments, the combination of the SIK2 inhibitor and the at least a first chemotherapeutic drug decreases expression of one or more genes involved in regulation of DNA repair and apoptosis in the cancer cell compared to cells treated with the SIK2 inhibitor or the at least a first chemotherapeutic drug alone.

In some embodiments, the one or more genes involved in regulation of DNA repair and apoptosis in the cancer cell are selected from BRCA2, EXO1, FANCD2, LIG4, XRCC4, BAX, BCL2, CASP7, and TRADD. In some embodiments, the one or more genes involved in regulation of DNA repair and apoptosis in the cancer cell are selected from EXO1, FANCD2, and XRCC4. In some embodiments, expression of the one or more genes is decreased by decreasing MEF2D binding to promoter regions.

In some embodiments, the type of cancer to be treated is a tumor with compromised homologous recombination (HR)-mediated DNA repair.

In some embodiments, the type of cancer to be treated as described herein is a BRCA1/2-mutant solid tumor.

In some embodiments, the type of cancer to be treated as described herein is a BRCA-independent tumor with compromised HR-mediated DNA repair.

In some embodiments, the treatment occurs outside of a clinical trial setting.

In some embodiments, a SIK2 inhibitor and at least a first chemotherapeutic drug as described herein may be administered in a clinical setting or may be administered in an alternate setting as deemed appropriate by a clinician or practitioner.

In some embodiments, administration of the SIK2 inhibitor in combination with one or more chemotherapeutic drugs to a patient inhibits growth of ovarian or breast cancer cells in the primary or recurrent cancer. In some embodiments, the cancer to be treated may be ovarian, endometrial, primary peritoneal, fallopian tube, and breast cancer Administration of the SIK2 inhibitor in combination with one or more chemotherapeutic drugs results in inhibition of growth of the cancer cells, or a reduction of tumor volume or size, or a reduction of symptoms associated with cancer.

In some embodiments, a SIK2 inhibitor in combination with one or more chemotherapeutic drugs as described herein may be combined with other therapies or treatments for cancer in a patient. Other drug treatments that may be used to treat cancer in combination with a SIK2 inhibitor and one or more chemotherapeutic drugs as described herein may include any chemotherapeutic drug and/or any immunotherapy drug known or available in the art. Any such drug treatments may be used as deemed appropriate by a clinician. A drug treatment that may be administered to a patient in combination with a SIK2 inhibitor and one or more chemotherapeutic drugs, e.g., carboplatin and/or paclitaxel, for treatment of cancer as described herein may include, but is not limited to, Evista (Raloxifene Hydrochloride), Raloxifene Hydrochloride, Soltamox (Tamoxifen Citrate), Tamoxifen Citrate, Abemaciclib, Abraxane (Paclitaxel Albumin-stabilized Nanoparticle Formulation), Ado-Trastuzumab Emtansine, Afinitor (Everolimus), Afinitor Disperz (Everolimus), Alkeran (Melphan), Alpelisib, Anastrozole, Aredia (Pamidronate Disodium), Arimidex (Anastrozole), Aromasin (Exemestane), Atezolizumab, Avastin (Bevacizumab), Bevacizumab, Capecitabine, Carboplatin, Cisplatin, Cyclophosphamide, Docetaxel, Doxorubicin Hydrochloride, Doxil (Doxorubicin Hydrochloride Liposome), Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara, (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Hycamtin (Topotecan Hydrochloride), Ibrance (Palbociclib), Infugem (Gemcitabine Hydrochloride), Ixabepilone, Ixempra (Ixabepilone), Kadcyla (Ado-Trastuzumab Emtansine), Keytruda (Pembrolizumab), Kisqali (Ribociclib), Lapatinib Ditosylate, Letrozole, Lynparza (Olaparib), Margenza (Margetuximab-cmkb), Margetuximab-cmkb, Megestrol Acetate, Melphalan, Methotrexate Sodium, Neratinib Maleate, Nerlynx (Neratinib Maleate), Niraparib Tosylate Monohydrate, Olaparib, Paclitaxel, Paclitaxel Albumin-stabilized Nanoparticle Formulation, Palbociclib, Pamidronate Disodium, Pembrolizumab, Perjeta (Pertuzumab), Pertuzumab, Pertuzumab, Rubraca (Rucaparib Camsylate), Trastuzumab, and Hyaluronidase-zzxf, Phesgo (Pertuzumab, Trastuzumab, and Hyaluronidase-zzxf), Piqray (Alpelisib), Ribociclib, Sacituzumab Govitecan-hziy, Soltamox (Tamoxifen Citrate), Talazoparib Tosylate, Talzenna (Talazoparib Tosylate),Tamoxifen Citrate, Taxol, Taxotere (Docetaxel), Tecentriq (Atezolizumab), Tepadina (Thiotepa), Thiotepa, Topotecan Hydrochloride, Toremifene, Trastuzumab, Trastuzumab and Hyaluronidase-oysk, Trexall (Methotrexate Sodium), Trodelvy (Sacituzumab Govitecan-hziy), Tucatinib, Tukysa (Tucatinib), Tykerb (Lapatinib Ditosylate), Verzenio (Abemaciclib), Vinblastine Sulfate, Xeloda (Capecitabine), Zejula (Niraparib Tosylate Monohydrate), Zoladex (Goserelin Acetate). Any other drugs known or available in the art may also be used in combination with a SIK2 inhibitor and one or more chemotherapeutic drugs as described herein without deviating from the scope of the present disclosure.

In some embodiments, a patient may be treated for ovarian cancer with a PARP inhibitor as described herein. Useful PARP inhibitors for treatment of ovarian cancer may include, but are not limited to, Olaparib, Rucaparib, and Niraparib. In some embodiments, a patient may be treated for breast cancer with a PARP inhibitor as described herein. Useful PARP inhibitors for treatment of breast cancer may include, but are not limited to, Olaparib and Talazoparib. In some embodiments, a SIK2 inhibitor described herein may be used to treat ovarian cancer in combination with one or more chemotherapeutic drugs, e.g., cisplatin, carboplatin, and/or paclitaxel, and a PARP inhibitor described herein. Useful SIK2 inhibitors for treatment of ovarian or breast cancer as described herein include, but are not limited to, Compound A or Compound B. In some embodiments, the SIK2 inhibitor is Compound A. In some embodiments, the SIK2 inhibitor is Compound B.

As would be understood by one of skill in the art, a SIK2 inhibitor and one or more chemotherapeutic drugs as described herein are administered in any form necessary or useful to the subject for treatment of cancer, for example, a liquid (e.g., injectable and infusible solutions), a semi-solid, a solid, an aqueous solution, a suspension, an emulsion, a gel, a magma, a mixture, a tincture, a powder, a capsule, a dispersion, a tablet, a pellet, a pill, a powder, a liposome, a lozenge, a troche, a liniment, an ointment, a lotion, a paste, a suppository, a spray, an inhalant, or the like. In some embodiments, a drug as described herein for treatment of cancer may be administered in a liquid or aqueous form for injection into a patient. The form can depend on the intended mode of administration and therapeutic application. Typically, compositions for the agents described herein are in the form of injectable or infusible solutions.

In some embodiments, a drug as described herein for treatment of cancer in a patient may be administered by any route or mode of administration, such as intravenous (IV), oral (p.o.), sublingual, rectal, vaginal, ocular, otic, nasal, cutaneous, enteral, epidural, intra-arterial, intravascular, nasal, respiratory, subcutaneous (s.c.), topical, transdermal, intramuscular, intra-peritoneal (i.p.), or the like. In some embodiments, a SIK2 inhibitor and one or more chemotherapeutic drugs as described herein are both administered orally.

In some embodiments, a SIK2 inhibitor described herein, such as Compound A or Compound B, blocks DNA double-strand break (DSB) repair in the cancer cells, preventing the cancer cells from repairing damage and thereby resulting in apoptosis (i.e., death) of the cancer cell. Blocking DSB repair by a SIK2 inhibitor as described herein increases nuclear localization of histone deacetylase (HDAC) 4/5, which increases nuclear localization of HDAC4/5 and blocks the activity of transcription factors associated with DNA DSB repair. In some embodiments, the transcription factor associated with DNA DSB repair as described herein is a myocyte enhancer factor-2 (MEF2) protein, such as MEF2D. Thus, in some embodiments, the combination of a SIK2 inhibitor and one or more chemotherapeutic drugs as described herein induces increased levels of apoptosis in the breast or ovarian cancer cells compared to cancer cells treated with only a SIK2 inhibitor or only with one or more chemotherapeutic drug.

In some embodiments, the combination of a SIK2 inhibitor and one or more chemotherapeutic drugs as described herein enhances the sensitivity of the breast or ovarian cancer cells to the chemotherapeutic or immunogenic drug described herein, such as carboplatin and/or paclitaxel. In some embodiments, the combination of a SIK2 inhibitor and a combination of chemotherapeutic drugs, such as a combination of carboplatin and paclitaxel as described herein enhances the sensitivity of the breast or ovarian cancer cells to the chemotherapeutic or immunogenic drug described herein. Therefore, the administration of the SIK2 inhibitor and the one or more chemotherapeutic drugs as described herein enhances the activity of other cancer treatment drugs. For example, in some embodiments, the combination of a SIK2 inhibitor and one or more chemotherapeutic drugs, e.g., carboplatin and/or paclitaxel, to treat ovarian or breast cancer enhances the anticancer activity of the SIK2 and/or the one or more chemotherapeutic drugs to produce a synergistic growth inhibition of the cancer cells. In some embodiments, the combination of the SIK2 inhibitor and one or more chemotherapeutic drugs decreases expression of one or more genes involved in regulation of DNA repair and apoptosis in the cancer cells compared to cells treated with either or any of the drugs in the combination alone. Any gene involved in regulation of DNA repair and apoptosis can be inhibited with a combination of a SIK2 inhibitor and one or more chemotherapeutic drugs as described herein, for example, one or more of BRCA2, EXO1, FANCD2, LIG4, XRCC4, BAX, BCL2, CASP7, and TRADD. Such genes are decreased or down-regulated by decreasing or eliminating the binding of a transcription factor (e.g., MEF2D) to the promoter regions of the genes, thereby decreasing expression of these genes, which results in the inability of the cancer cells to repair DNA, leading to apoptosis. In some embodiments, the combination of a SIK2 inhibitor and one or more chemotherapeutic drugs may decrease the expression or activity of EXO1, FANCD2, and XRCC4, which result in death of the ovarian or breast cancer cells as described herein.

Unless otherwise specified herein, the methods described herein can be performed in accordance with the procedures exemplified herein or routinely practiced methods well known in the art. The following sections provide additional guidance for practicing the methods of the present disclosure.

### Pharmaceutical Compositions

In some embodiments, a SIK2 inhibitor and one or more chemotherapeutic drugs may be administered together as a single composition, i.e., both or all drugs may be combined together in a solution or other drug form as described herein. In some embodiments, each drug may be administered separately (while still being administered concurrently), i.e., in separate solutions or drug forms as described herein. For example, a SIK2 inhibitor as described herein may be administered to a patient in an aqueous solution for intravenous administration, and one or more chemotherapeutic drugs may be administered in one or more separate or distinct aqueous solution(s) for intravenous administration. Pharmaceutical formulation is well established and known in the art.

In some embodiments, a SIK2 inhibitor and one or more chemotherapeutic drugs may be formulated with excipient materials, such as sodium citrate, sodium dibasic phosphate heptahydrate, sodium monobasic phosphate, Tween-80, and a stabilizer. The SIK2 inhibitor and one or more chemotherapeutic drugs can be provided, for example, in a buffered solution at a suitable concentration and can be stored at an appropriate temperature to maintain the efficacy of the drug(s), for example a temperature of 2-8° C. In some other embodiments, the pH of the composition is between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

A pharmaceutical composition described herein can also include agents that reduce aggregation of the drug when formulated. Examples of aggregation reducing agents include one or more amino acids selected from the group consisting of methionine, arginine, lysine, aspartic acid, glycine, and glutamic acid. The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

As described above for SIK2 inhibitors and chemotherapeutic drugs, compositions comprising these drugs can be administered by a parenteral mode (e.g., intravenous, subcutaneous, intraperitoneal, or intramuscular injection). In one embodiment, a composition comprising a SIK2 inhibitor and/or one or more chemotherapeutic drugs is administered intravenously. The phrases “parenteral administration” and “administered parenterally” as used herein mean modes of administration other than enteral and topical administration, usually by injection, and include, without limitation, intravenous, intramuscular, intra-arterial, intrathecal, intracapsular, intraocular, intracardiac, intradermal, intraperitoneal, transtracheal, subcutaneous, subcuticular, intraarticular, subcapsular, subarachnoid, intraspinal, epidural and intrasternal injection, and infusion.

A composition comprising a SIK2 inhibitor and/or one or more chemotherapeutic drugs can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, a composition comprising a SIK2 inhibitor and/or one or more chemotherapeutic drugs may be prepared with a carrier that will protect the components against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In some embodiments, a composition comprising a SIK2 inhibitor and/or one or more chemotherapeutic drugs is formulated in sterile distilled water or phosphate buffered saline. The pH of the pharmaceutical formulation may be between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

Administration of a SIK2 Inhibitor and/or Chemotherapeutic Drugs

A SIK2 inhibitor and/or one or more chemotherapeutic drugs as described herein can be administered to a subject, e.g., a patient in need thereof, by a variety of methods. For many applications, the route of administration is one of: intravenous injection or infusion (IV), subcutaneous injection (SC), intraperitoneally (IP), or intramuscular injection. Other modes of parenteral administration can also be used. Examples of such modes include: intra-arterial, intrathecal, intracapsular, intraocular, intracardiac, intradermal, transtracheal, subcuticular, intra-articular, subcapsular, subarachnoid, intraspinal, and epidural and intrasternal injection.

The route and/or mode of administration of the SIK2 inhibitor and/or one or more chemotherapeutic drugs, or compositions comprising these, can also be tailored for the individual case, e.g., by monitoring the patient.

The composition(s) comprising a SIK2 inhibitor and/or one or more chemotherapeutic drugs can be administered as a fixed dose, or in a mg/kg dose. The dose can also be chosen to reduce or avoid production of antibodies against the SIK2 inhibitor and/or one or more chemotherapeutic drugs. Dosage regimens are adjusted to provide the desired response, e.g., a therapeutic response or a combinatorial therapeutic effect. Generally, doses of the SIK2 inhibitor and/or one or more chemotherapeutic drugs (and optionally an additional agent) can be used in order to provide a subject with the agent in bioavailable quantities.

A SIK2 inhibitor and/or one or more chemotherapeutic drugs can be administered, e.g., at a periodic interval over a period of time (a course of treatment) sufficient to encompass at least 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 does, 20 doses, or more, e.g., once or twice daily, or about one to four times per week, or such as weekly, biweekly (every two weeks), every three weeks, monthly, e.g., for between about 1 to 12 weeks, such as between 2 to 8 weeks, such as between about 3 to 7 weeks, such as for about 4, 5, or 6 weeks, or every 5 weeks, or every 6 weeks, or any interval deemed appropriate by a clinician. Factors that may influence the dosage and timing required to effectively treat a subject, include, e.g., the stage or severity of the disease or disorder, formulation, route of delivery, previous treatments, the general health and/or age of the subject, and other diseases present. Moreover, treatment of a subject with a therapeutically effective amount of a SIK2 inhibitor and/or one or more chemotherapeutic drugs, or compositions comprising these, can include a single treatment or can include a series of treatments.

If a subject is at risk for developing a disorder described herein, the SIK2 inhibitor and/or one or more chemotherapeutic drugs can be administered before the full onset of the disorder, e.g., as a preventative measure. The duration of such preventative treatment can be a single dosage of the composition, or the treatment may continue (e.g., multiple dosages). For example, a subject at risk for the disorder or who has a predisposition for the disorder may be treated with a composition as described herein for days, weeks, months, or even years, so as to prevent the disorder from occurring or fulminating.

For patients receiving treatment for ovarian or breast cancer, resistance of the cancer cells to the SIK2 inhibitor and/or to the one or more chemotherapeutic drugs can reduce the efficacy of the drug(s). For these patients, administration of a combination of a SIK2 inhibitor and/or one or more chemotherapeutic drugs can increase the sensitivity of cancer cells to the SIK2 inhibitor and/or to the one or more chemotherapeutic drugs , thus prolonging the effects of the drugs and thereby prolonging the survival of the patient having cancer.

In some embodiments, a SIK2 inhibitor and/or one or more chemotherapeutic drugs may be administered to a patient in order to extend the duration of remission or to prevent a relapse or reduce the incidence of relapse of a cancer patient in remission.

A combination of a SIK2 inhibitor and/or one or more chemotherapeutic drugs can be administered to a patient in need thereof (e.g., a patient that has had or is at risk of having breast or ovarian cancer) alone or in combination with (i.e., by co-administration or sequential administration) other therapeutic treatments or drugs for treating cancer (e.g., additional (e.g., at least a second or third chemotherapeutic or immunotherapy drugs or treatments). In one embodiment, the additional therapeutic treatments or drugs are included in a pharmaceutical composition as described herein. In other embodiments, the additional therapeutic treatments or drugs are co-administered, administered concurrently, or administered sequentially in separate or distinct compositions.

### Kits

A SIK2 inhibitor and/or one or more chemotherapeutic drugs for treatment of breast or ovarian cancer in a patient can be provided in a kit. In one embodiment, the kit includes (a) a container that contains the SIK2 inhibitor and/or the one or more chemotherapeutic drugs as described herein, and optionally (b) informational material. The informational material can be descriptive, instructional, marketing or other material that relates to the methods described herein and/or the use of the agents for therapeutic benefit.

In one embodiment, the kit also includes additional agents (e.g., additional chemotherapeutic or immunotherapy drugs described herein) for treating cancer described herein. For example, the kit includes a first container that contains the SIK2 inhibitor and/or one or more chemotherapeutic drugs, and a second container that includes the chemotherapeutic or immunotherapy drug. In another embodiment, the kit includes a first container that contains the SIK2 inhibitor, a second container that contains the one or more chemotherapeutic drugs, and a third container that contains the additional chemotherapeutic or immunotherapy agent(s).

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the SIK2 inhibitor and/or the one or more chemotherapeutic drugs, as well as the additional chemotherapeutic or immunotherapy drug, e.g., in a suitable dose, dosage form, or mode of administration (e.g., a dose, dosage form, or mode of administration described herein), to treat a subject who has had or who is at risk for breast or ovarian cancer. The information can be provided in a variety of formats, include printed text, computer readable material, video recording, or audio recording, or information that provides a link or address to substantive material, e.g., on the internet.

In addition to the SIK2 inhibitor and/or the one or more chemotherapeutic drugs, and including any additional chemotherapeutic or immunotherapy drug(s) if applicable, the kit can include other ingredients, such as a solvent or buffer, a stabilizer, or a preservative. The SIK2 inhibitor, and and/or one or more chemotherapeutic drugs or immunotherapy drugs, can be provided in any form described herein, e.g., liquid, dried or lyophilized form, substantially pure and/or sterile. When the agents are provided in a liquid solution, the liquid solution is an aqueous solution. When the agents are provided as a lyophilized product, the lyophilized powder is generally reconstituted by the addition of a suitable solvent. The solvent, e.g., sterile water or buffer (e.g., PBS), can optionally be provided in the kit.

The kit can include one or more containers for the drugs or compositions. In some embodiments, the kit contains separate containers, dividers or compartments for the drugs and informational material. For example, the SIK2 inhibitor, and any chemotherapeutic or immunotherapy drugs, if applicable, can be contained in a bottle, vial, or syringe, and the informational material can be contained in a plastic sleeve or packet. In other embodiments, the separate elements of the kit are contained within a single, undivided container. For example, the SIK2 inhibitor, and chemotherapeutic or immunotherapy drugs, if applicable, are contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the SIK2 inhibitor, and the chemotherapeutic or immunotherapy drugs, if applicable, e.g., in a desired ratio. For example, the kit includes a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight.

The kit optionally includes a device suitable for administration of the SIK2 inhibitor, and chemotherapeutic or immunotherapy drugs, if applicable, e.g., a syringe or other suitable delivery device. The device can be provided pre-loaded with one or both of the agents or can be empty, but suitable for loading.

### Definitions

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Unless defined otherwise, all technical and scientific terms used herein have the meaning commonly understood by one of ordinary skill in the art to which the disclosure pertains. Specific terminology of particular importance to the description of the present disclosure is defined below.

As used in this specification and the appended claims, the singular forms “a,” “an,” and “the,” along with similar references used in the context of describing a particular embodiment (especially in the context of certain of the following claims), can be construed to cover both the singular and the plural, unless specifically noted otherwise. Thus, for example, “an active agent” refers not only to a single active agent, but also to a combination of two or more different active agents, “a dosage form” refers to a combination of dosage forms, as well as to a single dosage form, and the like. In some embodiments, the term “or” as used herein, including the claims, is used to mean “and/or” unless explicitly indicated to refer to alternatives only or the alternatives are mutually exclusive.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. In some embodiments, “about” refers to a specified value +/−10%.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

As used herein, “clinical response” is an indicator of therapeutic efficacy in combination with other indicators. In some embodiments, a clinical response refers to a percentage of patients whose cancer reduces, shrinks, lessens, etc. after treatment. For example, in some embodiments, a combination of a SIK2 inhibitor and one or more chemotherapeutic drugs may produce a 70% clinical response, indicating that 70% of patients administered such combination experienced a reduction in cancer after treatment.

As used herein, “co-administration” refers to the simultaneous administration of one or more drugs with another. In some embodiments, both drugs are administered at the same time. Co-administration may also refer to any particular time period of administration of either drug, or both drugs. For example, as described herein, a drug may be administered hours or days before administration of another drug and still be considered to have been co-administered. In some embodiments, co-administration may refer to any time of administration of either drug such that both drugs are present in the body of a patient at the same. In some embodiments, either drug may be administered before or after the other, so long as they are both present within the patient for a sufficient amount of time that the patient received the intended clinical or pharmacological benefits.

Conservative amino acid substitutions providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); Arginine (R), Lysine (K); 5) Isoleucine (I), Leucine (L), Methionine (M), Valine (V); and 6) Phenylalanine (F), Tyrosine (Y), Tryptophan (W). Not all residue positions within a protein will tolerate an otherwise “conservative” substitution. For instance, if an amino acid residue is essential for a function of the protein, even an otherwise conservative substitution may disrupt that activity, for example the specific binding of an antibody to a target epitope may be disrupted by a conservative mutation in the target epitope.

In some embodiments, conservative amino acid substitutions, e.g., substituting one acidic or basic amino acid for another, can often be made without affecting the biological activity of a recombinant polypeptide as described herein. Minor variations in sequence of this nature may be made in any of the peptides disclosed herein, provided that these changes do not substantially alter (e.g., by 15% or more) the desired activity of the protein.

As used herein, a dosage unit form or “fixed dose” as used herein refers to physically discrete units suited as unitary dosages for the patients to be treated; each unit contains a predetermined quantity of a SIK2 inhibitor and/or one or more chemotherapeutic drugs calculated to produce the desired therapeutic effect in association with the required pharmaceutical carrier and optionally in association with the other agent. Single or multiple dosages may be given. Alternatively, or in addition, the SIK2 inhibitor and/or one or more chemotherapeutic drugs, or composition(s) comprising these may be administered via continuous infusion.

A pharmaceutical composition(s) comprising a SIK2 inhibitor and/or one or more chemotherapeutic drugs as described herein may include a “therapeutically effective amount” of the SIK2 inhibitor and/or the one or more chemotherapeutic drugs as described herein. The term “therapeutically effective amount,” “pharmacologically effective dose,” “pharmacologically effective amount,” or simply “effective amount” may be used interchangeably and refers to that amount of an agent effective to produce the intended pharmacological, therapeutic or preventive result, e.g., a reduction of cancerous cells or lessened cancer cell burden (i.e., reduction in number of cancer cells), tumor size, tumor density, lymph node involvement, metastases, or associated symptoms in the patient. The pharmacologically effective amount results in the amelioration of one or more symptoms of a disorder (e.g., ovarian or breast cancer), or prevents the advancement of a disorder, or causes the regression of the disorder, or prevents the disorder. Such effective amounts can be determined based on the effect of the administered agent, or the combinatorial effect of agents if more than one agent is used. A therapeutically effective amount of an agent may also vary according to factors such as the disease stage, state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects. In some examples, an “effective amount” is one that treats (including prophylaxis) one or more symptoms and/or underlying causes of cancer. In one example, an effective amount is a therapeutically effective amount. In one example, an effective amount is an amount that prevents one or more signs or symptoms of a particular disease or condition from developing.

As used herein, “gene expression” or “expression” refers to the process of gene transcription, translation, and post-translational modification.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent Compound And approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt.

As used herein, “platinum resistance” or “platinum resistant” refers to a recurrence of cancer, e.g., ovarian cancer, in a patient within 6 months of completion of first-line platinum-based chemotherapy, such as treatment with a platinum-based chemotherapeutic drug, such as carboplatin, cisplatin, or oxaliplatin as described herein. In some embodiments, platinum resistance may refer to recurrence of cancer within 6 months of receiving treatment with multiple lines of chemotherapy. In some embodiments, platinum resistance refers to cancer, e.g., ovarian cancer, that initially responds to treatment with a platinum-based chemotherapeutic drug, but then recurs within a certain period, e.g., 6 months after treatment. In some embodiments, knowing whether a particular cancer is platinum-resistant may help plan further treatment.

Likewise, “platinum sensitivity” or “platinum sensitive” refers to a patient in which the amount of time that has elapsed between the completion of platinum-based treatment and the detection of relapse, known as the platinum-free interval (PFI), is a period of 6 months or more.

As used herein, “reducing” refers to a lowering or lessening, such as reducing cancer cell burden. In some embodiments, administration of a SIK2 inhibitor and/or one or more chemotherapeutic drugs as described herein may result in “reduced” or lessened cancer cell burden (i.e., reduction in number of cancer cells), tumor size, tumor density, lymph node involvement, metastases, or associated symptoms in the patient compared to a patient not been administered such drugs. “Reducing” may also refer to a reduction in disease symptoms as a result of a treatment as described herein, either alone, or co-administered with another drug.

As used herein, a “SIK inhibitor” refers to a compound, molecule, drug, etc., that inhibits the activity of salt-inducible kinase (SIK), which plays a role in several types of cancer, including ovarian cancer as describe herein. In some embodiments of the present disclosure, a “SIK inhibitor” may refer to an inhibitor of SIK1, SIK2, or SIK3. As would be known to one of skill in the art, some compounds, molecules, or drugs described herein may inhibit one of SIK1, SIK2, or SIK3, or may inhibit more than one, or all, of SIK1, SIK2, or SIK3. In some embodiments, a SIK inhibitor useful for the present disclosure in combination with at least a first chemotherapeutic drug may inhibit only SIK1, referred to herein as a SIK1 inhibitor, or may inhibit only SIK2, referred to as a SIK2 inhibitor, or may inhibit only SIK3, referred to herein as a SIK3 inhibitor. In some embodiments, a SIK inhibitor as described herein may be capable of inhibiting all of SIK1, SIK2, and SIK3.

As used herein, “subject” or “individual” or “patient” refers to any patient for whom or which therapy is desired, and generally refers to the recipient of the therapy. A “subject” or “patient” refers to any animal classified as a mammal, e.g., human and non-human mammals. Examples of non-human animals include dogs, cats, cattle, horses, sheep, pigs, goats, rabbits, etc. Unless otherwise noted, the terms “patient” or “subject” are used herein interchangeably. In some embodiments, a subject amenable for therapeutic applications may be a primate, e.g., human and non-human primates.

The terms “treating” and “treatment” or “alleviating” as used herein refer to reduction or lessening in severity and/or frequency of symptoms, elimination of symptoms and/or underlying cause, and improvement or remediation of damage. In certain aspects, the term “treating” and “treatment” as used herein refer to the prevention of the occurrence of symptoms. In other aspects, the term “treating” and “treatment” as used herein refer to the prevention of the underlying cause of symptoms associated with a disease or condition, such as breast or ovarian cancer. The phrase “administering to a patient” refers to the process of introducing a composition or drug into the patient via an art-recognized means of introduction. “Treating” or “alleviating” also includes the administration of compounds or agents to a subject to prevent or delay the onset of the symptoms, complications, or biochemical indicia of a disease (e.g., breast or ovarian cancer), alleviating the symptoms or arresting or inhibiting further development of the disease, condition, or disorder. Subjects in need of treatment include those already suffering from the disease or condition, as well as those being at risk of developing the disease or condition. Treatment may be prophylactic (to prevent or delay the onset of the disease or condition, or to prevent the manifestation of clinical or subclinical symptoms thereof) or therapeutic suppression, or alleviation of symptoms after the manifestation of the disease or condition.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1

### SIK2 Inhibition Sensitizes Ovarian and Breast Cancer Cells by Enhancing Olaparib-Mediated Inhibition of PARP Enzyme Activity

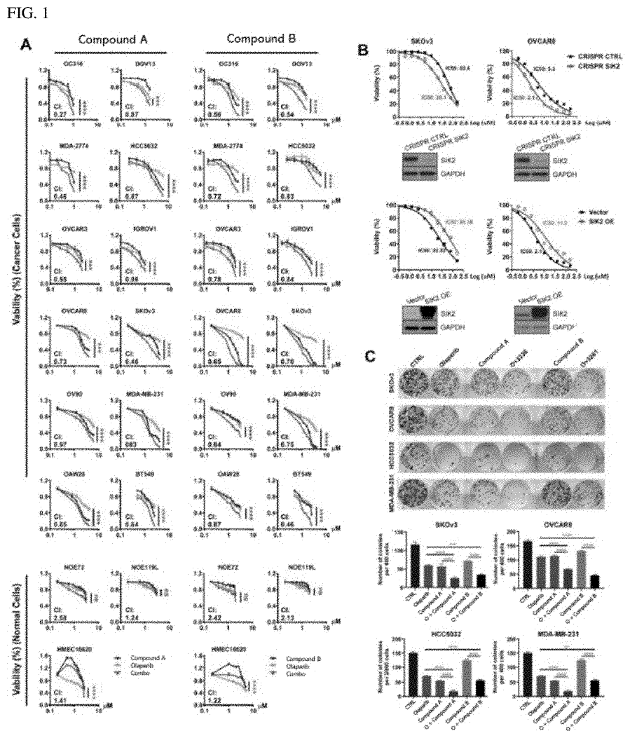

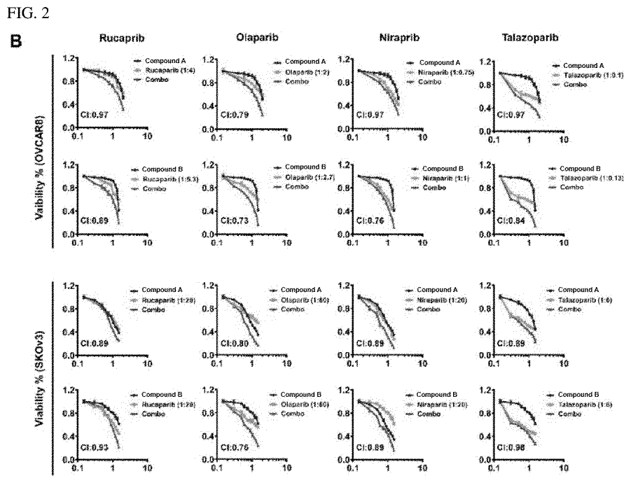

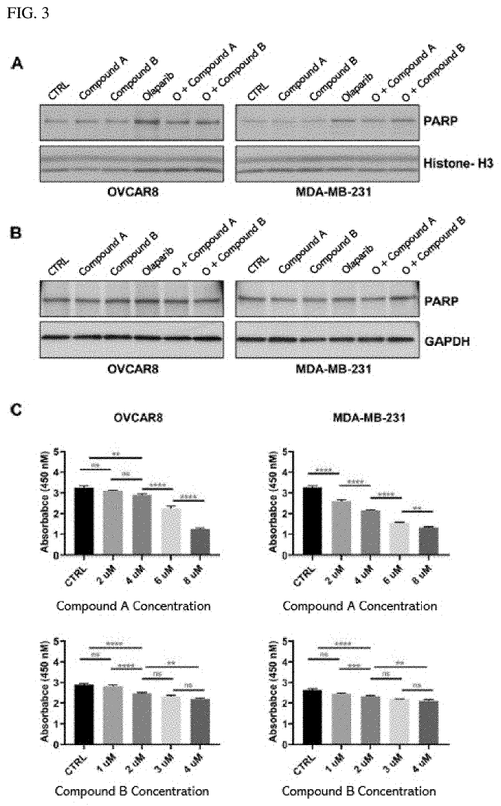

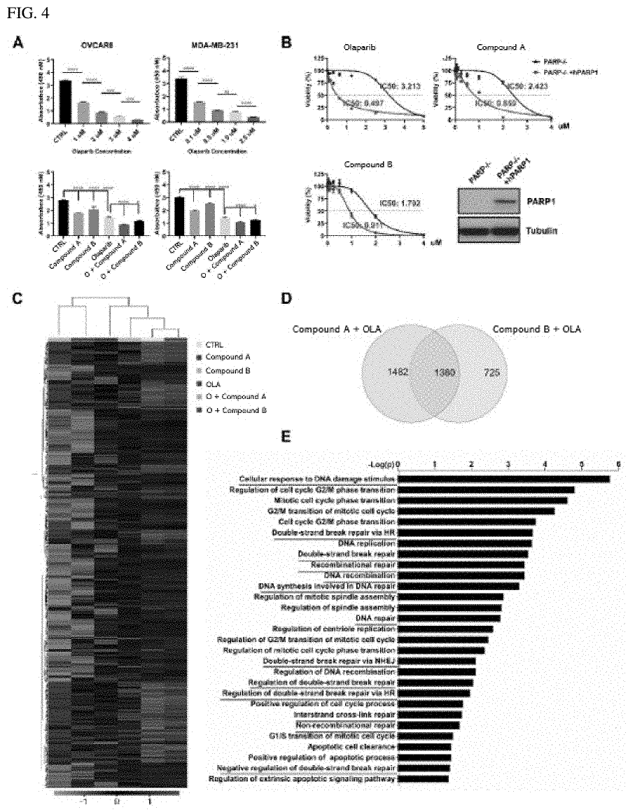

To explore whether modulation of SIK2 kinase activity can sensitize cancer cells to PARP inhibitors, the effect of combining a SIK2 kinase inhibitor (Compound A or Compound B) with olaparib was examined on cell growth in 10 ovarian and 2 triple-negative breast cancer cell lines as well as in normal cell lines (FIG. 1A). Sources and culture media for the cell lines described herein are provided in Table 1. Olaparib-induced growth inhibition (green line) was significantly enhanced by combination treatment (red line) with either Compound A or Compound B in all 12-cancer cell lines tested, but not in non-tumorigenic NOE72 and NOE119L (normal ovarian epithelial cells) and HMEC16620 (human mammary epithelial cells) (FIG. 1A). Moreover, all 12-cancer cell lines demonstrated synergistic growth inhibition with a combination of Compound A or Compound B with olaparib (combination index CI<1 using the CalcuSyn model), when compared to non-tumorigenic cells that did not undergo such a synergistic growth inhibition (FIG. 1A). To exclude potential off-target effects of SIK2 inhibitors, SIK2 was knocked down by CRISPR/Cas9 and stable ectopic expression of SIK2 was established in SKOv3 and OVCAR8 ovarian cancer cells. Knock-out of SIK2 sensitized cancer cells to olaparib judged by lower IC₅₀ (the concentration of a drug that gives half-maximal response, see Table 2) for olaparib in SIK2 deficient cells compared to control cells (FIG. 1B). In contrast, stable ectopic expression of SIK2 in SKOv3 and OVCAR8 cell lines desensitized cancer cells to olaparib, evidenced by an increased IC₅₀ of olaparib (FIG. 1B). Clonogenic assays were performed using three ovarian and one triple-negative breast cancer cell lines. Combination treatment with a SIK2 inhibitor and olaparib significantly decreased the number and size of colonies when compared to either the SIK2 inhibitor or olaparib alone (FIG. 1C and FIG. 2A). Furthermore, synergistic activity of SIK2 inhibition with PARP inhibition was evaluated with three structurally distinct PARP inhibitors (rucaparib, niraparib, and talazoparib) that have different PARP trapping potential. Although clinical PARP inhibitors can be ranked by their ability to trap PARP (from the most to the least potent): talazoparib>>niraparib>olaparib=rucaprib, SIK2 inhibitors synergized with PARP inhibitors with high (talazoparib) and low PARP trapping activity (olaparib) exhibiting similar combination indices (FIG. 2B). PARP binding in the chromatin fraction (indicative of PARP trapping) remained unchanged following treatment with SIK2 inhibitors, suggesting that SIK2 inhibitor-mediated enhancement of PARP inhibition is independent of PARP trapping activity (FIG. 3A). Measurement of PARP enzyme activity did, however, indicate that treatment with SIK2 inhibitors further decreased olaparib-induced suppression of PARP enzyme activity in cancer cells with detectable PARP protein levels (FIG. 4A and FIGS. 3B and 3C); consistent with the possibility that inhibition of PARP enzyme activity underlies the synergistic effect of SIK2 and PARP inhibition. To further test this possibility, DT40 PARP-1−/− cells that lack PARP enzyme activity (avian cells lack PARP2) were treated with SIK2 inhibitors or olaparib. DT40 PARP-1−/− cells resisted olaparib or SIK2 inhibitors, consistent with the lack of PARP1/2 (FIG. 4B). This is consistent with the synergistic effect of SIK2 inhibitors and olaparib depending upon the presence of PARP protein and PARP enzyme activity.

TABLE 1
Source and culture medium of cell lines.

| | | Culture | Tissue |
|---|---|---|---|
| Cell Line | Source | Medium | Classification |
| OC316 | Gordon Mills | RPMI 1640 | Ovarian Cancer |
| MDA-2774 | Gordon Mills | RPMI 1640 | Ovarian Cancer |
| OVCAR3 | Gordon Mills | RPMI 1640 | Ovarian Cancer |
| OVCAR8 | Gordon Mills | RPMI 1640 | Ovarian Cancer |
| OV90 | ATCC | 105 + 199 | Ovarian Cancer |
| OAW28 | ATCC | DMEM | Ovarian Cancer |
| DOV13 | Gordon Mills | DMEM | Ovarian Cancer |
| HCC5032 | Gordon Mills | RPMI 1640 | Ovarian Cancer |
| IGROV1 | Gordon Mills | RPMI 1640 | Ovarian Cancer |
| SKOv3 | ATCC | RPMI 1640 | Ovarian Cancer |
| MDA-MB-231 | ATCC | RPMI 1640 | Breast Cancer |
| BT549 | ATCC | RPMI 1640 | Breast Cancer |
| NOE72 | Robert Bast | 105 + 199 | Normal Ovarian |
| NOE119L | Robert Bast | 105 + 199 | Normal Ovarian |
| HMEC16620 | Robert Bast | 105 + 199 | Normal Breast |
| SKOv3 SIK2 KO | Ahmed Ahmed | RPMI 1640 | Ovarian Cancer |
| SKOv3 SIK2 KO | Ahmed Ahmed | RPMI 1640 | Ovarian Cancer |
| CTRL | | | |
| OVCAR8 SIK2 KO | Ahmed Ahmed | RPMI 1640 | Ovarian Cancer |
| OVCAR8 SIK2 KO | Ahmed Ahmed | RPMI 1640 | Ovarian Cancer |
| CTRL | | | |
| SKOv3 SIK2 OE | Ahmed Ahmed | RPMI 1640 | Ovarian Cancer |
| SKOv3 SIK2 OE | Ahmed Ahmed | RPMI 1640 | Ovarian Cancer |
| CTRL | | | |
| OVCAR8 SIK OE | Robert Bast | RPMI 1640 | Ovarian Cancer |
| OVCAR8 SIK2 OE | Robert Bast | RPMI 1640 | Ovarian Cancer |
| CTRL | | | |

TABLE 2
IC₅₀ values of inhibitors and concentration ration of SIK2
inhibitors to Olaparib.

| | Com- | Com- | | Com- | Com- |
|---|---|---|---|---|---|
| | pound A | pound B | Olaparib | pound | pound |
| | (μM) | (μM) | (μM) | A: | B: |
| Cell Line | (IC₅₀) | (IC₅₀) | (IC₅₀) | Olaparib | Olaparib |
| OVCAR8 | 1.396 | 1.765 | 6.396 | 1:0.5 | 1:0.5 |
| SKOv3 | 1.231 | 2.843 | 55.51 | 1:12.5 | 1:12.5 |
| HCC5032 | 2.243 | 2.397 | 14.52 | 1:3 | 1:1 |
| OC316 | 1.259 | 2.25 | 4.91 | 1:5 | 1:2 |
| IGROV1 | 1.084 | 1.605 | 2.83 | 1:5 | 1:2 |
| MDA-2774 | 1.081 | 0.592 | 6.289 | 1:5 | 1:2 |
| OV90 | 3.869 | 5.428 | 8.364 | 1:1 | 1:2 |
| DOV13 | 2.811 | 1.995 | 32.16 | 1:6 | 1:3 |
| OVCAR3 | 2.828 | 2.29 | 3.788 | 1:1 | 1:1 |
| OAW28 | 1.714 | 3.400 | 3.658 | 1:1 | 1:1.3 |
| MDA-MD-231 | 2.369 | 1.182 | 4.474 | 1:0.5 | 1:0.5 |
| BT549 | 2.02 | 2.1 | 13.7 | 1:4 | 1:4 |
| NOE72 | 5.745 | 4.161 | 6.383 | 1:0.5 | 1:0.75 |
| NOE119L | 4.805 | 5.860 | 15.06 | 1:0.5 | 1:0.75 |
| HMEC16620 | 3.6 | 5.3 | 1.2 | 1:0.5 | 1:0.25 |

### Example 2

### Compound A and Compound B Perturb Transcription of DNA Repair and Apoptosis Genes

While treatment of SIK2 inhibitors can enhance olaparib-mediated inhibition of PARP enzyme activity, it was asked whether SIK2 inhibitors might alter other key functional components of the DNA DSB repair pathways that might also contribute to the synergy observed between SIK2 and PARP inhibition. To explore this possibility, RNA-sequencing (RNA-seq) data was generated from perturbed SKOv3 cells and differential expression analysis was performed. The numbers of transcripts up or down-regulated by ≥2-fold after treatment with Compound A, Compound B, olaparib, Compound A+olaparib or Compound B+olaparib were 1308, 366, 3, 2862 and 2105, respectively. Based on a heatmap with unsupervised hierarchical clustering of 3587 transcripts altered by both Compound A+olaparib and Compound B+olaparib treatments (FIG. 4C), olaparib-treated and control groups shared relatively similar transcriptomes, whereas both SIK2 inhibitor and olaparib combination treatment groups clustered together. These data indicate that combination treatments showed the most significant alteration of transcripts compared to single agents alone and that SIK2 inhibition significantly induced olaparib-mediated transcriptional repression. (FIG. 4C). Using a Venn analysis, 1380 differentially expressed transcripts were shared by both SIK2 inhibitors and the olaparib combination treatment groups (FIG. 4D). Gene Ontology (GO) Biological Processes enrichment analysis of 1380 differentially expressed genes identified multiple aspects of regulation involving mitosis, DNA damage checkpoint, cell cycle, DNA repair and apoptosis (FIG. 4E), suggesting that SIK2 inhibition may enhance olaparib sensitivity by regulating DNA repair and apoptosis.

### Example 3

### Compound A and Compound B Enhance Olaparib-Induced DNA DSB and Apoptosis

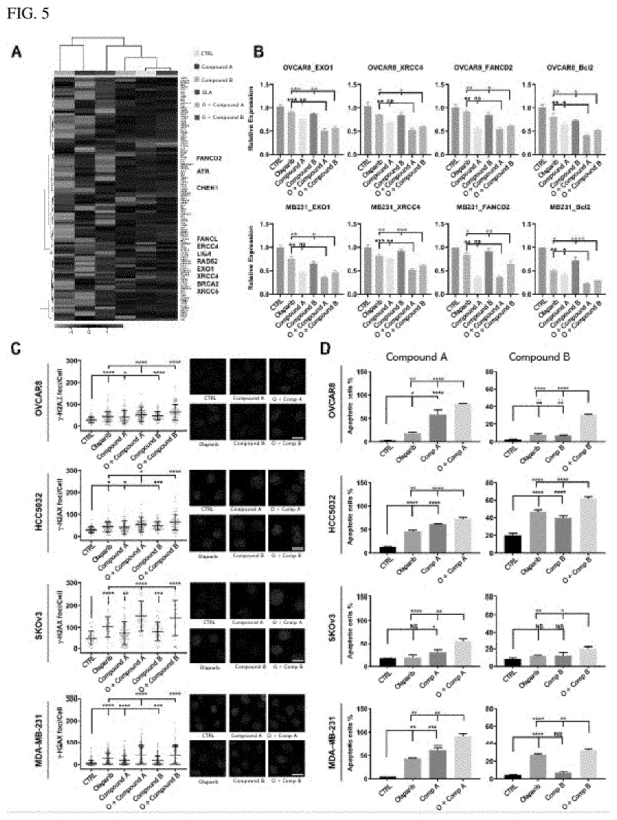

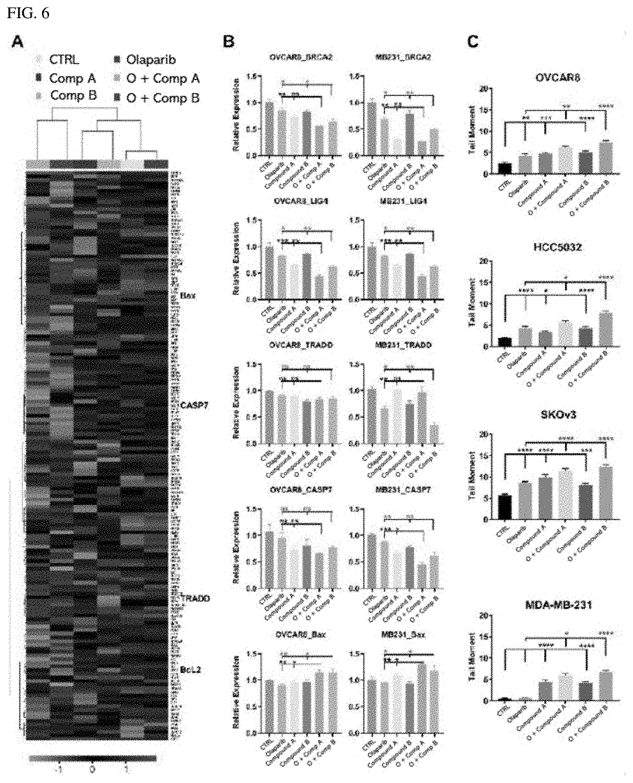

Detailed analysis of the expression of transcripts participating in regulation of DNA repair and apoptosis further demonstrated that SIK2 inhibition enhances PARP inhibition-mediated DNA repair (FIG. 5A) and apoptosis (FIG. 6A). To verify the RNA-seq results, nine genes involved in regulation of DNA repair and apoptosis (BRCA2, EXO1, FANCD2, LIG4, XRCC4, BAX, BCL2, CASP7, and TRADD) were selected and analyzed with RT-qPCR (quantitative reverse transcription PCR) using OVCAR8 ovarian cancer and MDA-MB-231 breast cancer cells. Treatment with Compound A or Compound B combined with olaparib (Compound A+olaparib or Compound B+olaparib) significantly decreased the expression of EXO1, XRCC4, FANCD2, BRCA2, LIG4, CASP7, and BCL2 and increased expression of BAX compared to olaparib treatment alone in both cell lines tested (FIG. 5B and FIG. 6B). Similar results were also observed in the cells treated with Compound B in combination with olaparib (FIG. 5B and FIG. 6B). These data are consistent with the observations documented in RNA-seq analysis.

To confirm whether SIK2 inhibitors induce DNA damage in cancer cells by inhibiting DNA repair, the effect of SIK2 inhibitors on olaparib-mediated induction of DNA DSBs was tested. Compound A, Compound B, or olaparib modestly increased levels of both phosphorylation of H2AX (γ-H2AX) and tailed DNA biomarkers, whereas combined treatment of SKOv3, OVCAR8, HCC5032, or MDA-MB-231 cells with Compound A or Compound B and olaparib increased the levels of γ-H2AX and the percentage of tailed DNA significantly (FIG. 5C and FIG. 6C), consistent with the possibility that SIK2 inhibition blocked DNA DSB repair. As unrepaired DSB can trigger apoptosis, Annexin V expression was measured to determine whether the combination of SIK2 inhibitor and olaparib induced greater levels of apoptosis. Compound A or Compound B combined with olaparib treatment induced significantly higher levels of apoptosis than did either single agent (FIG. 5D), consistent with the critical prerequisite of DNA DSB repair for cancer cell survival. Together, these results suggest that preventing DNA DSB repair by SIK2 inhibitors enhances the vulnerability of cancer cells to PARP inhibition.

### Example 4

### SIK2 Inhibition Decreases Phosphorylation of Class IIa HDACs and Promoter Activity of MEF2 Transcription Factors

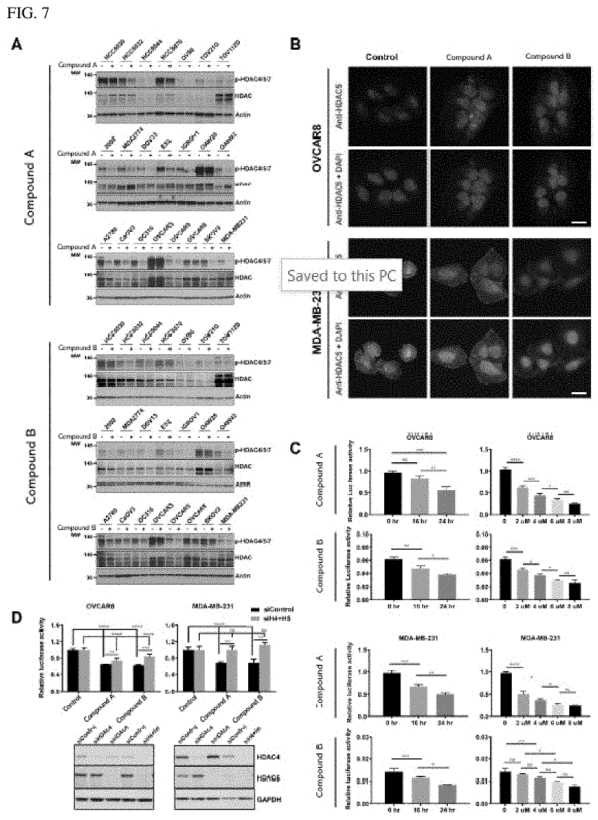

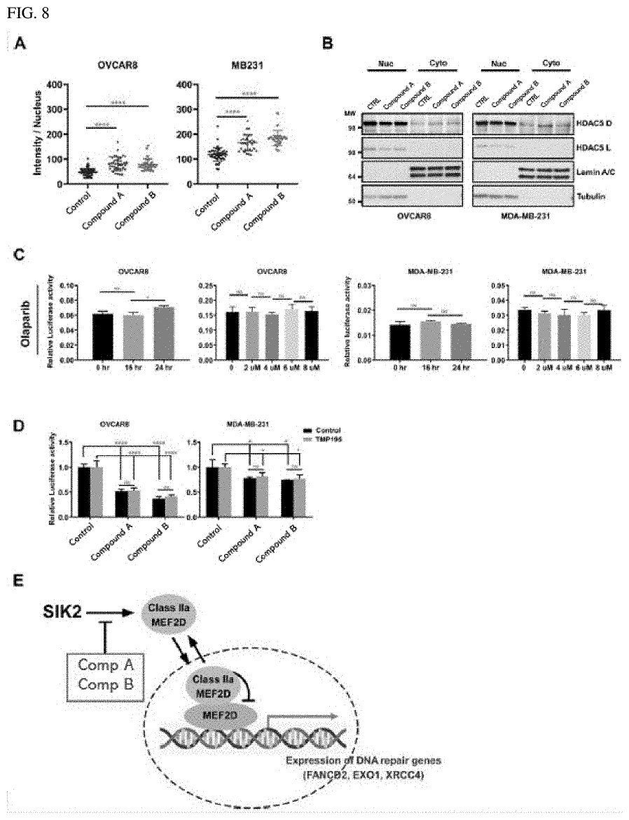

To identify the mechanism(s) by which SIK2 inhibition decreases DNA DBS repair, it was tested whether SIK2 inhibitors decrease the phosphorylation of class IIa HDACs, which control its nuclear cytoplasmic shuttling and consequently its association with DNA. Compound A or Compound B significantly decreased the phosphorylation of HDAC4 (Ser246)/HDAC5 (Ser256)/HDAC7 (Ser155) in nearly all the cell lines tested by western analysis using an antibody recognizing all three phosphorylation sites simultaneously (FIG. 4A). Next, it was investigated whether SIK2 inhibitors increase nuclear localization of HDAC5. SIK2 inhibition increased nuclear localization of HDAC5 judged by increasing nuclear florescence intensity (FIG. 7B and FIG. 8A) and the nuclear fraction of HDAC5 expression (FIG. 8B). This result raised the possibility that SIK2 inhibition downregulates expression of DNA repair genes by enhancing binding of HDAC5 with DNA-binding transcriptional factors, for which HDAC5 may serve as a transcriptional corepressor complex blocking the expression of MEF2 downstream targets. Therefore, it was hypothesized that SIK2 inhibition may block MEF transcription factor activity. To test this hypothesis, MEF2 promoter activity was measured using a luciferase reporter assay in ovarian and breast cancer cell lines, in the presence and absence of the SIK2 inhibitors Compound A, Compound B, or olaparib. SIK2 inhibitors significantly reduced MEF2 promoter activity in a time- and dose-dependent manner (FIG. 7C), but olaparib did not, as expected (FIG. 6C). Next, it was examined whether SIK2 regulation of MEF2 activity was HDAC4/5-dependent, increasing its binding to MEF2D protein. Knockdown of class IIa HDAC4/5 with siRNA prevented a Compound A or Compound B-mediated decrease of MEF2 promoter activity (FIG. 7D), but a decrease in MEF2 promoter activity was not prevented by inhibition of HDAC enzyme activity using TMP195, a selective class-IIa HDAC inhibitor (FIG. 8D). These observations are consistent with the hypothesis that SIK2 inhibition increases nuclear localization of HDAC4/5, blocking MEF2 transcription (FIG. 8E).

### Example 5

### SIK2 Inhibition Alters MEF2D Transcription Factor-Mediated Downstream Signaling

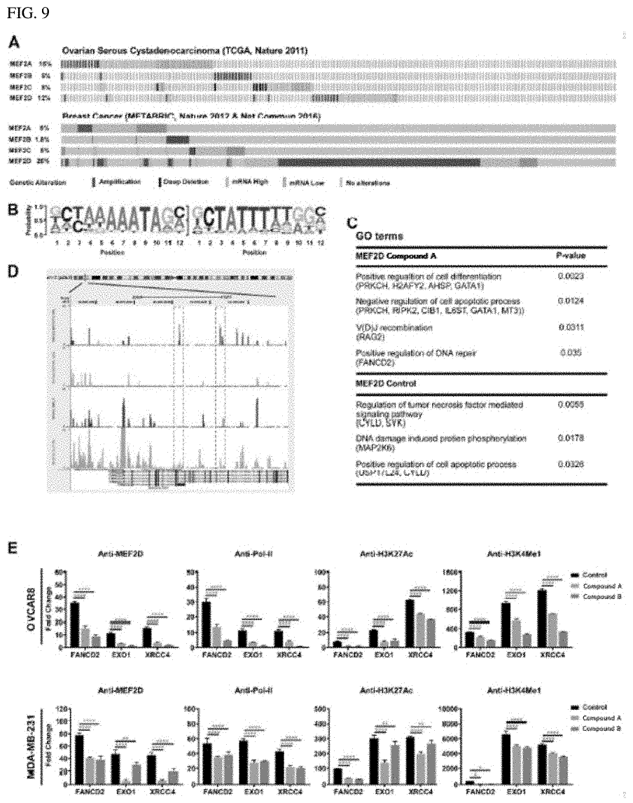

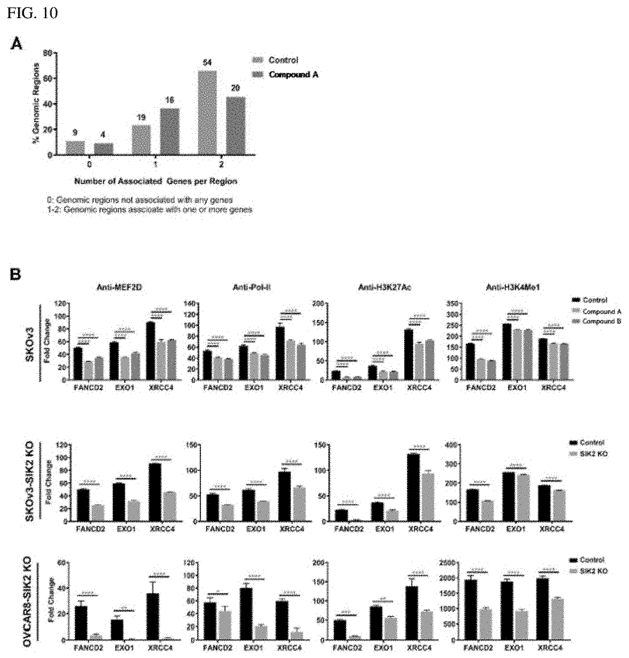

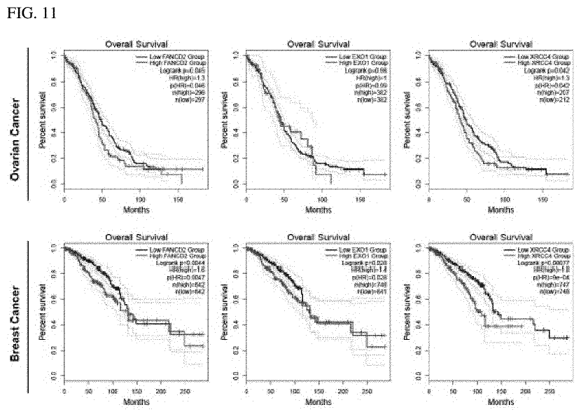

To explore the clinical relevance of the MEF2 transcription factors in ovarian and triple-negative breast cancers, alterations in the frequencies of individual MEF2 family members were examined in these tumor types. According to the cBioPortal TCGA database, 15-21% of ovarian and breast cancers contained amplification and mRNA upregulation of MEF2D (FIG. 9A). Genome-wide binding of MEF2D in SKOv3 ovarian cancer cells was then examined using chromatin immunoprecipitation sequencing (Chip-seq). In the genome-wide setting, 73 binding sites of MEF2D were identified and showed 50% reduction (36 binding sites) in the cells treated with Compound A (FIG. 10A). To identify a MEF2D consensus recognition sequence in ovarian cancer cells, de novo-motif discovery analysis was performed. A known MEF2 consensus recognition sequence could be detected in 59% (p=1e−9) of all random peaks analyzed (FIG. 9B). Moreover, motifs containing the consensus sequence for other TFs including Sox15, Usf2, and Sp1 were found at frequencies ranging from 19% to 34% suggesting that MEF2D can affect expression of downstream targets by associating with MEF2D DNA-binding site or by interacting with other transcription factors. This result is consistent with previous studies that have suggested MEF2D may function as a transcription factor or enhancer. In addition, GO enrichment analysis indicated that MEF2D-bound genes in control SKOv3 cells exhibited significant enrichment in positive regulation of cell differentiation, negative regulation of cell apoptotic processes, V(D) recombination and positive regulation of DNA repair. By contrast, several MEF2D-bound genes involved in regulation of the tumor necrosis factor mediated signaling pathway, DNA damage induced protein phosphorylation and positive regulation of cell apoptotic process were documented in cells treated with Compound A (FIG. 9C). Moreover, Chip-seq analysis indicated that MEF2D binds directly to FANCD2. FANCD2 plays a major role in homology-dependent repair (HDR)-mediated replication restart and in suppressing new origin firing. Chip-qPCR of FANCD2 confirmed MEF2D-association with the FANCD2 promoter/enhancer region. This association was decreased with SIK2 inhibition by Compound A or Compound B in all four cell lines assessed (FIG. 9E and FIG. 10B). Exonuclease I (EXO1) and X-ray repair cross-complementing protein 4 (XRCC4) are both downregulated by SIK2 inhibition in RNA-seq (FIG. 5A). EXO1 participates in extensive DSB end resection, an initial step in the homologous recombination (HR) pathway and XRCC4 is a component of the complex that mediates nonhomologous end-joining (NHEJ). Although EXO1 and XRCC4 genes were not associated with MEF2D peaks by Chip-seq analysis, which may due to poor quality of MEF2D antibody, the potential MEF2D binding sites at their promoter regions were identified. Chip-qPCR analysis revealed MEF2D binding to EXO1 and XRCC4 promoter/enhancer regions, and MEF2D binding affinities to those targets were significantly decreased with SIK2 inhibition by Compound A or Compound B in all cell lines tested (FIG. 9E and FIG. 10B). Notably, SIK2 inhibition also reduced H3K27Ac and H3K4Me1 RNA Pol-II at the FANCD2, EXO1, and XRCC4 promoter/enhancer regions (FIG. 9E and FIG. 10B). Both H3K27Ac and H3K4me1 are the activation marks of enhancers and have regulatory function to increase the transcription of target genes. PoI-II also is reported to regulate gene transcription by binding to both promoters and enhancers. Thus, these data support that FANCD2, EXO1, and XRCC4 are the direct targets of MEF2D and that SIK2 regulates DNA DSB repair by repression of MEF2D transcriptional activity. To evaluate the clinical relevance of the study, Kaplan-Meier survival analysis was examined, which showed that breast and ovarian patients with high expression of FANCD2 and XRCC4 have poorer overall survival than those with low expression of FANCD2 and XRCC4 (FIG. 11). EXO1 expression was also positively correlated with survival in breast cancer, but not in ovarian cancer (FIG. 11). These data are consistent with previous reports that overexpression of SIK2 correlates with poor prognosis in patients with ovarian and breast cancer.

### Example 6

### Overexpression of MEF2D is Sufficient to Block SIK2 Inhibition-Induced DNA Damage and Growth Inhibition

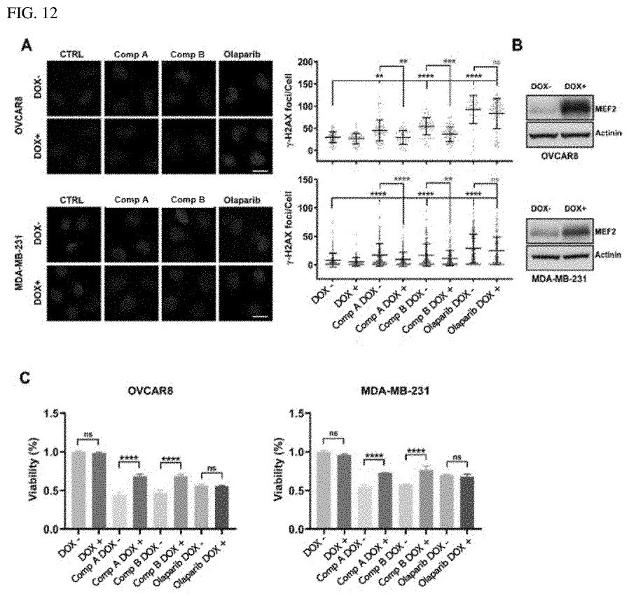

As describe above, SIK2 inhibition blocks HDAC4/MEF2-mediated DNA DSB repair by downregulating the expression of critical factors participating in this process. To test whether MEF2D downregulation was sufficient to explain the effects of SIK2 inhibition on DNA DSB repair and whether overexpression of MEF2D will rescue SIK2 inhibitor-mediated DNA damage and growth inhibition, OVCAR8 and MDA-MB-231 doxycycline (DOX)-inducible stable cell lines expressing MEF2D were generated. When MEF2D expression was induced by DOX treatment, γ-H2AX foci were significantly decreased in the cells treated with either Compound A (p<0.001 in MDA-MB-231 and p<0.0001 in OVCAR8 cells) or Compound B (p<0.0001 in both MDA-MB-231 and OVCAR8 cells), but not olaparib, compared to un-induced cells with no DOX treatment in both the OVCAR8 (p=0.4514) and MDA-MB-231 (p=0.3511) cell lines (FIGS. 12A and 12B). These data further confirm a role for MEF2D in promoting cancer survival by decreasing DNA damage in cancer cells. In addition, when viability was measured, induction of MEF2D partially rescued toxicity from Compound A or Compound B, but not from olaparib to cells with MEF2D induction (FIG. 12C). Together, these results suggest that SIK2 inhibitors enhance the vulnerability of cancer cells to olaparib not only by inhibiting PARP enzyme activity but also by blocking the class-IIa HDAC/MEF2D-mediated DNA repair function.

### Example 7

### Co-Administration of SIK2 Inhibitor and Olaparib is Synergistic In Vivo

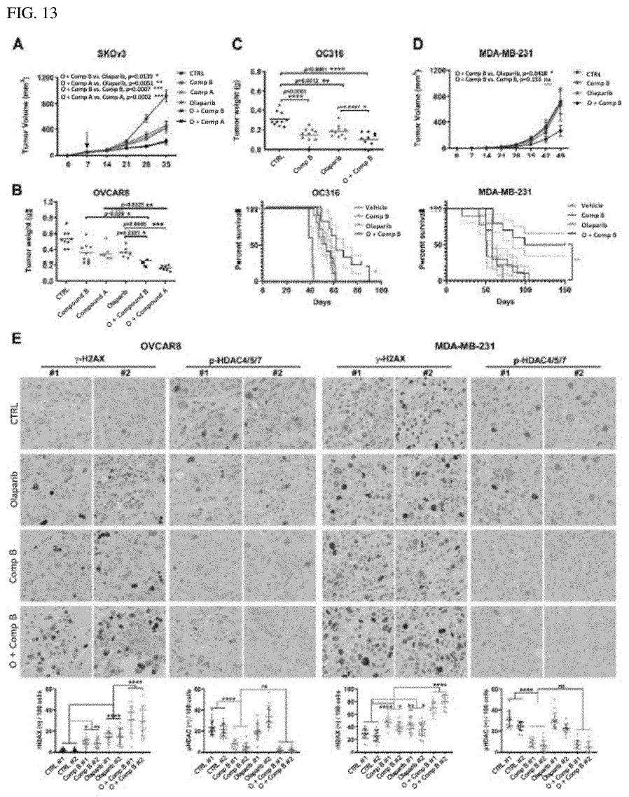

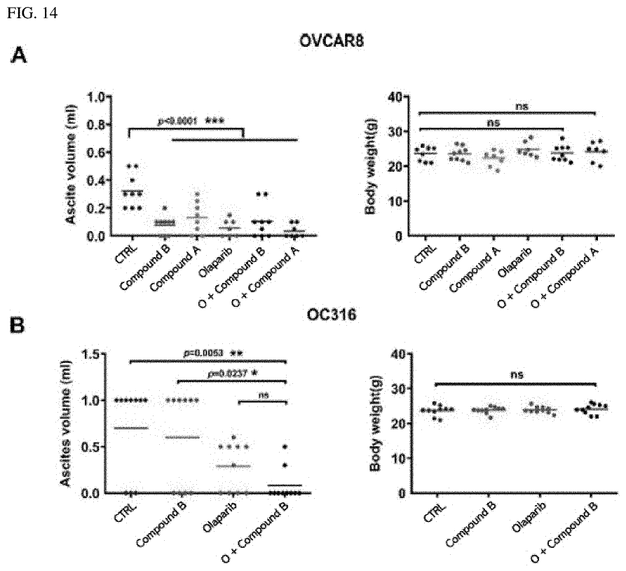

Based on enhancement of PARP inhibitor activity by SIK2 inhibition in cell culture, it was tested whether the addition of SIK2 inhibitors could promote PARP inhibitor response in vivo. When the BRCA-proficient SKOv3 cell line was injected subcutaneously into mice, treatment with Compound A, Compound B, or olaparib alone significantly inhibited tumor growth, compared to a vehicle control (FIG. 13A). The combination of Compound A+olaparib or Compound B+olaparib produced greater inhibition of tumor growth than either single agent (FIG. 13A). Another BRCA-proficient OVCAR8 ovarian cancer cell line was injected intraperitoneally into mice that were treated as described for the SKOv3 xenograft model. Compound A or Compound B in combination with olaparib combination significantly inhibited OVCAR8 tumor growth to a much greater degree than either single agent (FIG. 13B). In the OVCAR8 intraperitoneal xenograft model, Compound A or Compound B in combination with olaparib decreased formation of ascites. Moreover, the combination was well tolerated, with no significant weight loss compared to vehicle control (FIG. 14A). In addition, the OC316 (heterozygous BRCA2 mutated) ovarian cancer xenograft model was used to extend results observed with SKOv3 and OVCAR8 xenografts. Similar results were observed in the OC316 xenograft model (FIG. 13C and FIG. 14B). More importantly, the Compound B and olaparib combination prolonged survival compared to either agent alone, with tumor regression in 2 out of 10 xenografts (p<0.05) (FIG. 13C). To demonstrate relevance to breast cancer, xenografts with the BRCA-proficient TNBC cell line model MDA-MB-231 were studied. To reflect the original microenvironment, MDA-MB-231 cells were implanted directly into the mammary fat pad of female nude mice. One week after cell inoculation, mice were treated with single agent Compound B, olaparib, or the combination, and tumor volume was measured at the indicated intervals (FIG. 13D). Following treatment with either single agent Compound B or oalparib, tumor burden remained unchanged; however, the combination treatment inhibited tumor volume from day 28 and induced tumor regression in 5 of 10 mice (p<0.01) (FIG. 13D).

Tumors growing as xenografts were collected for histology with H&E and IHC staining. Routine H&E staining detected high-grade ovarian cancer in ovarian cancer xenograft models and breast cancer morphology in the breast cancer xenograft model, respectively. IHC of OVCAR8 and MDA-MB-231 xenograft tumors at study termination recapitulated in vitro studies. Compound B increased nuclear γ-H2AX staining, which was further increased by treatment with Compound B in combination with olaparib (p<0.0001) (FIG. 13E). Nuclear p-HDAC5 staining was decreased in Compound B treated tumors (p<0.0001), but not in olaparib treated tumors (FIG. 13E). These data are consistent with the notion that SIK2 inhibition enhances olaparib sensitivity through increasing nuclear localization of class IIa HDACs, decreasing MEF2D-mediated expression of DNA repair genes and increasing DNA damage. Taken together, these pre-clinical models demonstrate that SIK2 provides a novel target that could contribute to care of women with high-grade ovarian cancer and triple-negative breast cancer patients.

### Example 8

### Discussion

The present study documents for the first time that inhibition of SIK2 synergistically enhances sensitivity of high grade serous ovarian and triple-negative breast cancers to PARP inhibitors in cell culture and xenograft models. Synergistic activity was noted in BRCA mutant and wild type cancers. A novel mechanism underlies this synergistic interaction. A decrease of PARP enzyme activity and phosphorylation of class-IIa HDAC 4/5/7 mediate the effects of SIK2 inhibitors on tumor cell growth in ovarian and breast cancers. They were also necessary and sufficient for the synergy observed between SIK2 inhibitors and PARPi. Inhibition of the phosphorylation of class-IIa HDAC 4/5/7 by Compound A or Compound B SIK2 inhibitor, 1) abolishes class-IIa HDAC 4/5/7-associated transcriptional activity of MEF2, 2) decreases MEF2D binding to regulatory regions with high-chromatin accessibility in DNA repair genes, and 3) represses the critical gene expression in DNA DSB repair pathway. Decreased expression of FANCD2, RAD51, and XRCC4 due to SIK2 inhibition likely contributes to PARPi sensitivity through a MEF2D-dependent mechanism.

SIK2 inhibition decreased phosphorylation of class-IIa HDACs and increased nuclear localization of class-IIa HDAC proteins. Phosphorylation of class-IIa HDACs controls their signaling-dependent nucleocytoplasmic shuttling. Under basal conditions, class-IIa HDACs are unphosphorylated and located in the nucleus, where they are recruited to their target genes through interaction with transcription factors, enabling their transcriptional repressive function. Class-IIa HDACs become phosphorylated in response to specific signals, leading to disruption of the interaction with transcription factors, their export to the cytoplasmic compartment, and de-repression of their targets. A member of Class-IIa HDACs was thought to be a component of the DNA damage response, recruited to the same dots, or repair foci, together with 53BP1 which is vital in promoting NHEJ. It was demonstrated that SIK2-rgulation of the MEF2D-mediated DNA repair pathway depends upon SIK2-mediated phosphorylation of Class-IIa HDACs. Thus, Class-IIa HDACs appear to be the key regulators of the synergy observed between SIK2 inhibitors and PARP inhibitors.

MEF2 transcription factors have a diversity of functions in a wide range of tissues and have been implicated in several diseases. The spectrum of genes regulated by MEF2 in different cell types depends upon extracellular signaling and on co-factor interactions that modulate MEF2 activity. The MEF2 domain is also involved in interactions with co-activators and co-repressors. Co-repressors that are thought to associate with the MEF2 domains of all MEF2 family proteins include the class IIa histone deacetylases HDAC4, -5, -7 and -9. According to the cBioPortal database, 6 to 21% of ovarian serous cystadenocarcinomas, invasive breast cancer, lung squamous cell and adenocarcinomas, uterine endometriod carcinomas, stomach adenocarcinomas, adrenocortical carcinomas, esophageal carcinomas, bladder urothelial carcinomas and pancreatic adenocarcinomas contain amplified MEF2 genes. The present study documents for the first time that MEF2 genes may act as oncogenes by regulating expression of genes involved in DNA DSB repair in ovarian and breast cancer. SIK2 inhibition decreased MEF2 gene promoter activity and repressed expression of critical genes in the DNA DSB repair pathway, supporting the notion that Compound A and Compound B enhance sensitivity to PARPi by decreasing MEF2's oncogenic function.

Synergetic interaction of SIK2 inhibitors and PARP inhibitors was observed with three structurally distinct PARP inhibitors (rucaparib, niraparib, and talazoparib) that have differential PARP trapping potential. Combinations of SIK2 inhibitors with PARP inhibitors of higher PARP trapping potential (Talazoparib) and with lower PARP trapping activity (olaparib) produced similar combination indexes, consistent with comparable synergy. Measurement of PARP enzyme activity indicated that the SIK2 inhibitors enhanced the effect of olaparib by further decreasing PARP enzyme activity in cancer cells with detectable PARP protein levels. Furthermore, 2 different SIK2i demonstrated synergy with PARPi, consistent with on-target effects of SIK2i. PARPi elicit significant responses in BRCA1 or BRCA2 mutation carriers with breast, ovarian, prostate, and pancreatic tumors. Thus, developing new strategies to enhance PARPi sensitivity and expand the utility of PARPi to DNA DSB repair competent tumors is crucial.

This study has a number of limitations. We have demonstrated that Olaparib-induced growth inhibition was significantly enhanced by combination treatment with either Compound A or Compound B in all 12 cancer cell lines tested, but not in non-tumorigenic ovarian and mammary epithelial cells. Although it was potentially due to different levels of replication stress and ongoing DNA damage between normal and malignant cells, this mechanism has not yet been confirmed and demonstrated functionally in the cell lines studied. It has been revealed that decreased expression of FANCD2, RAD51, and XRCC4 due to SIK2 inhibition likely contributes to PARPi sensitivity through a MEF2D-dependent mechanism, however, MEF2 regulates the expression of many molecules, there may be additional effects of MEF2D that contribute to sensitization to PARPi by in cooperation with downregulation of FANCD2, RAD51 and XRCC4. The in vivo data are strongly supportive of efficacy and low toxicity of SIK2i and PARPi combination in patients.
Together, SIK2 inhibition decreases PARP enzyme activity and the expression of FANCD2, RAD51, and XRCC4, suggesting that the combination of SIK2i and PARPi has the potential to increase the magnitude and duration of PARPi activity in patients with different cancers. Thus, future clinical trials could be designed to determine whether the combination will benefit these patients. The present animal studies, particularly with Olaparib and Compound A/Compound B, did not show significant toxicity based on weight loss. The potential for tolerability in patients is further supported by the lack of synergism of the combination in the normal cell lines. PARP inhibitors are now approved for ovarian, breast, and prostate cancers. Compound B has exhibited minimal hematologic toxicity during toxicology studies and has been cleared by the FDA to initiate a phase I trial to find the maximum tolerated dose (MTD) of Compound B alone and in combination with paclitaxel in ovarian cancer. Assessing the combination of PARPi and SIK2i in the clinical setting should therefore be prioritized to optimize the use of these compounds and to maximize patient benefit.

### Example 9

### Study Design

The objective of this study was to define the effect of SIK2i (Compound A and Compound B) on cancer cell growth in ovarian and triple-negative breast cancers, as well as to explore the synergy between SIK2 and PARP inhibition. It was demonstrated that SIK2 inhibition synergistically enhanced PARP inhibitor activity in a variety of ovarian and triple-negative breast cancer cell lines and xenograft models. In vitro experiments were performed in biological triplicate unless otherwise stated. Sample sizes were determined on the basis of previous experience and was sufficient to detect statistically significant differences between treatments. For in vivo experiments, mice were randomly assigned to treatment groups. Experiments were not blinded. Study groups were followed until individual tumor measurements reached 1.5 cm in diameter, at which point sacrifice was indicated in accordance with Institutional Animal Care and Use Committee protocols.

### Example 10

### Statistical Analysis

Experiments were repeated two or three times. Data were plotted using GraphPad Prism 8 and compared using two-tailed student t test and one-way or two-way ANOVA test. Kaplan-Meier survival analysis of xenograft studies was performed using Log Rank test by GraphPad. Data are presented as Mean±STD unless specified. p<0.05 is considered significant. *p<0.05, **p<0.01, ***p<0.001 and ****p<0.0001.

### Example 11

### Cell Lines

Cell lines used in this study are listed in Table 1. The identity of all cell lines was confirmed with STR DNA fingerprinting in the MDACC Characterized Cell Line Core (supported by NCI P30CA016672). All cell lines were maintained in a 5% CO₂ incubator at 37° C. and mycoplasma tested with a Universal Mycoplasma Detection Kit from ATCC.

### Example 12

### Viability Assays

Cell viability was determined using CellTiter-Glo® Luminescent Cell Viability Assay (Promega). 2000-4000 Cells were plated in 96-well plates and treated with a SIK2 inhibitor (Compound A or Compound B) and a PARP inhibitor (Rucaparib, Niraparib, olaparib or Talazoparib) alone or combined in serial dilutions 24 hrs after seeding. After 5-days of incubation, media were removed and a mixture of 30 uL of CellTiter-Glo reagent and 60 uL of culture media was added to each well. Luminescence was measured on a Synergy2 microplate reader (BioTek) after 10 min of shaking. Dose-response experiments were plotted and IC₅₀ values were calculated using nonlinear curve fitting with normalized response and variable slope by GraphPad Prism 8. Drug interaction of the two-drug combination using a constant ratio were processed and a Combination Index (CI) was calculated using CalcuSyn 2.0 (BIOSOFT). CI<1 indicates synergism, CI=1 indicates additive effect and CI>1 indicates antagonism.

### Example 13

### Clonogenic Assays

Individual cells were seeded in 6-well plates in triplicate at the density of 200, 400 or 600 cells/well depending on doubling time. Cells were treated with single or double agents at different concentrations 1 day after seeding. Cells were grown up to two weeks until visible colonies were formed. Culture media with different treatments were refreshed every other day. At the conclusion of the experiment, cells were washed twice with PBS, fixed in 0.1% Brilliant Blue R with 10% v/v acetic acid and 30% v/v methanol for 1 min and washed with tap water until background was clear. Pictures were taken using a FluoChem E Imager. Clones with >50 cells were counted.

### Example 14

### PARP Trapping Assay

Chromatin extraction was performed as described by Muvarak and colleagues using a subcellular protein fractionation kit (Thermo Scientific, 78840) (48). Briefly, Pellets were first lysed in membrane extraction buffer. Nuclei were then lysed in nuclear extraction buffer to isolate a nuclear soluble fraction. The remaining chromatin (nuclear insoluble) fraction was washed once with nuclear extraction buffer, then digested with 300 units of micrococcal nuclease to release chromatin-bound proteins. PARP binding in the chromatin fraction (indicative of PARP trapping) was assayed by Western blot analysis of the chromatin cell fraction against the PARP antibody.

### Example 15

### PARP Enzyme Activity Assay

PARP enzyme activity assay. PARP enzyme activity was measured using a PARP universal colorimetric assay kit (R&D system, 4677-096-K). Cells were plated and treated with Compound A (6 μM)/Compound B (4 μM), Olaparib (0.05 μM), and a combination of both for 26 hrs on different ovarian cancer cell lines. Cell lysates were collected using cell extraction buffer. The biotinylated poly (ADP-ribose) deposited by PARP-1 in cell lysates onto immobilized histones in a 96-well plate was detected. Streptavidin-HRP (biotin-binding protein) and a colorimetric HRP substrate were added to produce relative absorbance that correlates with PARP-1 activity.

### Example 16

### Chromatin Immunoprecipitation (ChIP) and RT-qPCR Analysis

OVCAR8, MDA-MB-231, SKOv3, OVCAR8-SIK2 KO or SKOv3-SIK2 KO cells (2 million) were cultured on a 150-cm plate, and treated the next day either with vehicle control or with Compound A (4 μM) or Compound B (5 μM) for 48 hrs. Chip assays were performed using the Magna Chip A Kit (Millipore). Briefly, cells after treatment with Compound A or Compound B for 48 hrs were incubated with 1% formaldehyde for 10 min at room temperature and neutralized with 1× glycine. Nuclei were isolated and sonicated to obtain 200-1000 bp DNA fragments using the QSONICA sonicator for 30 cycles with 10 seconds pulses at 100% amplitude with 2 min of incubation on ice between pulses. For individual ChIP assay, 100m of soluble chromatin per sample was immunoprecipitated with 8 μg of mouse IgG control antibody (Santa Cruz, sc-2025), 8 μg of rabbit control antibody (Millipore, PP64B), 8 μg of MEF2D antibody (Santa Cruz, sc-27115 3X), 8 μg of RNA polymerase II antibody (Abcam, ab817), 6 μg of Histone H3 (acetyl K27) antibody (Abcam, ab45173) or 6 μg of Histone H3 (tri methyl K4) (Abcam, ab8580). For ChIP-Sequence, 500 μg of chromatin per sample was immunoprecipitated with 40 μg of MEF2D antibody. Input determined from 1% of the cell lysate was used as a negative control. Purified and enriched DNA was quantified using real time quantitative PCR (RT-qPCR) with the following primers. FANCD2D Forward, 5′-ACC TGT TAT GAG CGT GAA GTC-3′ (SEQ ID NO:1) and Reverse, 5′-GAT GCA GGA CTG TGC ATT AGA-3′ (SEQ ID NO:2); EXD2 Forward, 5′-GGT CTG GCC TAA GGT TTC TTC-3′ (SEQ ID NO:3) and Reverse, 5′-CAG TTC ACG CTG GGT TCT T-3′ (SEQ ID NO:4); and XRCC Forward, 5′-GCA GTC TTC CTA GTC TCA ACT-3′ (SEQ ID NO:5) and Reverse, 5′-TTG CCC TTC TAG GAG CTT AAT G-3′ (SEQ ID NO:6). RT-qPCR was performed using iTaq Universal SYBR Green Supermix (Bio-Rad, 172-5124) in a CFX Connect RT-qPCR (Bio-Rad). Thermal cycling condition was as follows: 94° C. for 10 min, followed by 40 cycles of 94° C. for 20 sec, and 60° C. for 60 sec. Analysis of qPCR data was calculated using fold enrichment method (The ChIP signals are divided by the IgG antibody signals, 2^{-DDCt}).

### Example 17

### ChIP-Sequence and Analysis

Sequencing was performed by the Sequencing and Microarray Facility (SMF) at MD Anderson Cancer Center. Briefly, Indexed libraries were prepared from 20 ng of Diagenode Biorupter sheared ChIP DNA using the KAPA Hyper Library Preparation Kit (Kapa Biosystems, Inc). Libraries were amplified by 8 cycles of PCR and then size distribution was assessed using the 4200 TapeStation High Sensitivity D1000 ScreenTape (Agilent Technologies) and quantified using the Qubit dsDNA HS Assay Kit (ThermoFisher). The indexed libraries were multiplexed, 10 libraries per pool. The pool was quantified by qPCR using the KAPA Library Quantification Kit (KAPA Biosystems) then sequenced on the Illumina NextSeq500 sequencer using the High-output 75 single read configuration. The raw reads were first preprocessed to remove sequencing adapters and low quality reads. The trimmed reads were then mapped to human reference genome hg19 using bowtie, with only uniquely mapped reads retained. The ChIP-seq occupancy profiles were generated by MACS 1.4 with the “—wig” parameter, and were normalized to 20 million total reads. Duplicated reads were automatically removed by MACS. ChIPseq peaks were called by MACS with p-value set to 1e−8. Peaks were annotated to associated genes according to their relative locations. T associated genes were identified as ChIP-seq target genes. Further functional analysis on these genes were carried out, including gene ontology (GO) analysis using DAVID. The enriched DNA binding motifs in ChIP-seq peak regions were identified and compared with known motifs using HOMER v4.8.

### Example 18

### mRNA-Sequence and Analysis

Poly(A)-containing mRNA sequencing was performed by Sequencing and ncRNA program at MD Anderson cancer center. The indexed mRNA sequencing libraries were prepared from total RNA with RIN>9.0 using Illumina TrueSeq stranded mRNA library preparation kits (Illumina, RS-122-2101 and RS-122-2102), following guidance of an Illumina Truseq stranded mRNA protocol. In Brief, 200 ng of total RNA were used for poly(A) mRNA enrichment using oligo(dT) coated magnetic beads. The enriched and purified mRNA was fragmented into small pieces using divalent cations at elevated temperature. The cleaved RNA fragments were then reverse transcribed into first strand cDNA by reverse transcriptase using random hexamer primers for RT priming and reverse transcription, followed by second strand cDNA synthesis using DNA polymerase I and RNase H. These double strand cDNA fragments were end-repaired and then adenylated at 3′ ends with the addition of a single ‘A’ base to prevent self-ligation during subsequent ligation to the illumina index-specific adapters that has a single “T” at 3′ end which provides complementary overhang for ligating the adapter to the fragment. The raw library products were purified and enriched by PCR to create the final cDNA sequencing library. The indexed individual sequencing library was quantified using an Agilent Bioanalyzer High Sensitive DNA assay. To ensure the sufficient data coverage for high, medium and low copy transcripts, twelve indexed mRNA libraries were pooled and sequenced on an Illumina Nextseq 500 sequencer using TruSeq High Output Kit V2 150 cycles (FC-404-2001) in Paired-end E75 sequencing configuration. The raw data bcl files were de-multiplexed and converted into fastq file by using Illumina bcl2fastq2 conversion V 2.19 software (illumina). We used FastQC to perform a quality control of the FASTQ files and STAR (GRCh38, Gencode25 and STAR 2.6.1b) to map the reads against the reference genome and count the number of reads uniquely mapping to each gene, for each sample. Heatmap plots of selected genes showing their variation among different samples were generated in R, version 3.5.1, using the heatmap 2 function of g plots library. Public domain of gene pathways (qiagen.com/us/) was used to retrieve genes related to apoptosis and DNA Damage repair. Gene ontology enrichment analysis for differentially expressed genes was performed using the web-based tool Enrichr.

### Example 19

### Immunoblot

Cells were incubated with and without treatment for the intervals indicated and then cells were incubated in lysis buffer (50 mM Hepes, pH 7.0, 150 mM NaCl, 1.5 mM MgCl₂, 1 mM EGTA, 10, 10% glycerol, 1% Triton X-100, 50 mM NaF, 1 mM Na₃VO₄ 1 mM PMSF, 10 μg/mL leupeptin and10 μg/mL aprotinin) on ice for 30 min. Lysates were centrifuged at 15,000 g at 4° C. for 15 min, and supernatants were collected. To prepare subcellular fractions of nuclear soluble and chromatin-bound material, cells were treated with indicated drugs, and then cells were collected by scraping and subsequent centrifugation at 4° C. For fractionation, we used a Subcellular Protein Fractionation kit (Thermo Scientific, 78835) following the manufacturer's instructions. The protein concentration was assessed using a bicinchoninic acid (BCA) protein assay (Thermo Scientific, 23228). The proteins were separated by SDS-PAGE and transferred to Polyvinylidene difluoride (PVDF) membranes (Thermo Scientific, 88518). After being blocked with 5% BSA in TBST (tris-buffered saline with 0.1% tween 20 detergent), the membranes were incubated with primary antibodies at 4° C. overnight, followed by 1:2000 horseradish peroxidase (HRP)-conjugated secondary antibody (Thermo Scientific, anti-mouse 3439 and anti-rabbit 31463) for 40-60 min at room temperature. Bands were visualized using an ECL Western Blotting Substrate (PerkinElmer, NEL 104001EA). SIK2 (CST6919), p-HDAC4/5/7 (CST3443), HDAC5 (CST20458), HDAC4 (CST5392) and actin (CST4967) antibodies were purchased from Cell Signaling Technology. GAPDH (MAB374) antibody is from Millipore. PARP (551052) and MEF2D (610775) antibodies are from BD Pharmingen. Lamin A/C (sc-6215) antibody is Santa Cruz. Actinin (CBL-231) antibody is from Chemicon and α-Tubulin (T9026) antibody is from Sigma.

### Example 20

### RNA Extraction and RT-qPCR Analysis

Cells were treated with and without Compound A or Compound B for 72 hrs and lysed in TRIzol (ThermoFisher, 15596026). Total RNA was extracted using an RNeasy kit (Qiagen, 217004) according to the manufacturer's instructions. cDNA was synthesized from 2 μg of RNA using the Superscript II First Strand Synthesis Kit (Invitrogen, 11904-018). RT-qPCR was performed using CFX Connect Real-time System (Bio-Rad) in a total volume of 20 μL, which included 10 μL of 2× SsoAdvanced Universal PCR master (PCR primers are included) and 5 ng of cDNA. Thermal cycling conditions were as follows: 95° C. for 2 min, followed by 40 cycles of 95° C. for 5 sec, and 60° C. for 30 sec. PrimePCR Custom Plates (96 well) which contain 2× SsoAdvanced Universal PCR master mix and PCR primers were custom ordered from Bio-Rad. Data were analyzed by the ΔΔCT method using GAPDH as a housekeeping gene. Experiments were run in triplicate.

### Example 21

### Establishment of OVCAR8 and SKOv3 SIK2 CRISPR/Cas9 Knock Out Cell Lines

OVCAR8 and SKOv3 SIK2 knock out cell lines were established using CRISPR/Cas9 technology known in the art. Briefly, a plasmid with GFP containing Cas9 and the sgRNA expression were transfected to cancer cells. CRISPR-mediated knockout was performed using guide RNAs targeting exon 2 (AATAATCGATAAGTCTCAGC, SEQ ID NO:7) and exon 4 (GATTTTCAGCTTTGAGGTCA, SEQ ID NO:8). Transfected cells were isolated by FACS for single-cell culture 2-3 days after transfection, and then the cells were expanded and harvested for detection of the protein expression using western analysis.

### Example 22

### Establishment of OVCAR8 and MDA-MB-231 MEF2D inducible cell lines.

OVCAR8 and MDA-MB-231 cells were infected with pLV(Exp)-Neo-CMV>tTS/rtTA_M2 lentivirus (VectorBuilder, VB160419-1020mes) and subsequently selected using 1 μg/mL of G418 according to the manufacturer's protocol (Dharmacon). Clonal populations were generated by limiting dilution under G418 (Corning 61-8833-100mg) selection. OVCAR8 and MDA-MB-231 cells with clonal population of CMV>tTS/rtTA were again infected with pLV(Tet)-EGFP:T2A:Puro-TRE-hMEF2D lentivirus (VectorBuilder, VB180504-1036gtn). Clonal populations were generated by limiting dilution under puromycin (Sigma, D-9897-1G) selection. Clones with the best expression efficiency were selected by western blotting under 1 μg/mL doxycycline (Sigma, D-9897-1G) for 48 hrs. OVCAR8-MEF2D and MDA-MB-231-MEF2D inducible cells were maintained in RPMI 1640 (Corning, 15-040-CV) supplemented with 10% FBS, G418 (1000 μg/mL for MDA-MB-231 and 500 μg/mL for OVCAR8) and puromycin (2 μg/mL for MDA-MB-231 and 1 μg/mL for OVCAR8).

### Example 23

### RNA Interference

ON-TARGETplus pooled siRNAs targeting human HDAC4 (J-003497), HDAC5 (J-003498) and Non-targeting Control siRNA #2 (D-001810-02) and DharmaFect 4 (T-2004-03) were purchased from GE Dharmacon. 70 nM of siRNA and 0.2% DharmaFECT 4 were diluted in OPTI-MEM medium individually and then mixed together for 20 min at room temperature. Cells were then laid on top of siRNA-DharmaFECT mixture. Cells were lysed to determine target gene expression and prepared for luciferase activity assay 72 hrs post transfection (see Luciferase Reporter Assay below).

### Example 24

### Immunohistochemical Staining (IHC)

Formalin fixed and paraffin embedded mouse tissue sections were deparaffinized and rehydrated in gradient ethanol solutions. Antigens were retrieved in Rodent Decloaker (BioCare Medical, RD913M) and microwaved twice in an EZ Retriever System V3 (BioGenex) at 95° C. for 5 min. Tissues were blocked in PeroxAbolish (BioCare Medical, PXA969M) for 30 min, Rodent Block M (BioCare Medical, RBM961L) for 30 min, and 5% BSA in PBS for 30 min. Tissues were incubated with primary antibody as indicated overnight at 4° C. VisUCyte HRP Polymer IgG (R&D Systems, VC001-025 for mouse, VC003-025 for rabbit) was applied for 30 min at room temperature followed by DAB chromogenesis (BioCare Medical, BDB2004L). Tissues were counter-stained with CAT hematoxylin (Thermo Fisher, CATHE-M) for 20 sec. The slides were then dehydrated through gradient ethanol solutions and two passes of xylene and sealed with Permount (Thermo Fisher, SP15-100).

### Example 25

### Luciferase Reporter Assay

MEF2 promoter activity was quantified using an MEF2 reporter assay Kit (QIAGEN, 336841 CCS-7024L). Cells were plated and after overnight incubation transfected with a mixture of a MEF2-responsive luciferase vector and a constitutively expressing Renilla luciferase vector (40:1) for 24 hrs. Cells were re-plated into a 96 well plate, incubated for 16 hrs and then treated with Compound A (4 μM) or Compound B (4 μM) for different intervals or with different doses of Compound A and Compound B for 24 hrs as indicated. Cells were then lysed for a dual luciferase assay. The relative luciferase activity of MEF2 was calculated by normalizing to Renilla luciferase activity. To quantify MEF2 promoter activity with and without knockdown of HDAC4 and HDAC5, cells were transfected with targeting siRNA or control siRNA for 24 hrs prior to transfection of a mixture of a MEF2-responsive luciferase and Renilla luciferase vectors. Cells were re-plated into a 96 well plate and then treated with Compound A (4 μM) or Compound B (4 μM) for 24 hrs. HDAC4 and HDAC5 siRNA knockdown efficiency was measured by western blot analysis.

### Example 26

### Alkaline Single-Cell Agarose Gel Electrophoresis (Comet) Assays

1-2×10⁵ cells in 6-well plates were treated with DMSO, SIK2 inhibitor (Compound A and Compound B), olaparib or the combination of SIK2 inhibitor and olaparib. Treatment conditions were as follows: 1 μM of Compound A for HCC5032, OVCAR8 and SKOv3 and 0.5 μM of Compound A for MDA-MB-231 for 48 hrs; 5 uM of Compound B for all four cell lines for 48 hrs; and 5 μM of olaparib for all four cell lines for 16 hrs before harvest. Cells were trypsinized and resuspended at 2×10⁵/mL in cold PBS without Ca²⁺ and Mg²⁺. Cells were mixed with pre-warmed comet agarose at 1:10 (v/v) ratio. 10 uL of cell agarose mixture was plated onto comet slides pre-coated with 75 uL of agarose and chilled at 4° C. for 15 min to set. Cells were lysed in 25 mL of Lysis buffer at 4° C. for 2 hrs and washed with alkaline solution (pH 10). Comet slides were electrophoresed in cold alkaline solution at 20V for 15 min. Slides were rinsed with water and dried in 70% ethanol for 5 min. Slides were then stained with Vista Green DNA dye and viewed using an Olympus epifluorescence microscope with a FITC filter. Images were captured using a 20× objective. 3-Well OxiSelect™ Comet Assay kit are from Cell Biolabs, Inc (STA-351). Experiments were run in triplicate and Olive Tail Moment was measured using CaspLab1.2.3β2 software (CaspLab.com). Olive Tail Moment=Tail DNA %×Tail Length. 50-200 Cells were measured for each treatment and experiments were repeated twice independently to ensure reproducibility.

### Example 27

### Immunofluorescence Staining

Cells on 22×22 mm coverslips were fixed in 4% formaldehyde in PBS (Thermo Fisher, J19943-K2) and permeabilized with 0.1% Triton X-100 (Sigma, X100) in PBS for 15 min. Cells were blocked with 5% BSA in PBS for 30 min and then stained with antibody overnight at 4° C., followed by secondary antibody and DAPI for 1 hr. Coverslips were mounted with Fluoro-Gel with TES buffer (Electron Microscopy Sciences, 50-246-96) and air dried. HDAC5 nuclear localization was evaluated by measuring nuclear fluorescence intensity of HDAC5. Cells were treated with DMSO, Compound A (3 μM) or Compound B (5 μM). After 24-hrs incubation, cells were fixed in 4% formaldehyde in PBS. Cells were stained as described above. Images were captured using an Olympus Model IX71 measuring nuclear HDAC4 fluorescence intensity in each cell using ImageJ (imagej.nih.gov/ij/). DNA damage visualized by γ-H2AX staining was evaluated by counting nuclear γ-H2AX puncta in each cell. Cells were treated with DMSO, 1 μM of olaparib alone, 4 μM of Compound B or 1 μM of Compound A, or the combination of olaparib and SIK2 inhibitors. After 8 hrs incubation, cells were fixed in 4% formaldehyde in PBS. Cells were stained as described above. Images were captured using an Olympus IX71 microscope and nuclear γ-H2AX puncta in each cells were counted using with Olympus CellSens Dimension software. HDAC5 (CST20458) and γ-H2AX (CST2577) antibodies were purchased from Cell Signaling Technology. Experiments were repeated twice independently to ensure reproducibility and 50-200 cells were counted for each treatment.

### Example 28

### Apoptosis

The percentage of apoptotic cells induced by Compound A/Compound B, olaparib, or a combination of both were measured on different ovarian cancer cell lines by fluorescence activated cell sorting (FACS) using FITC Annexin V/Dead cell Apoptosis Kit I (Thermo Fisher, cat. V13242) according to the manufacturer's instructions. Briefly, following indicated treatment, cells were harvested and washed once in 1× PBS. Afterward, cells were resuspended in 1× binding buffer containing 5 uL of fluorochrome-conjugated Annexin V plus 100 μg/ml PI (Propidium iodide) After 15 mins incubation at room temperature cells were centrifuged and resuspended in 200 μl 1× binding buffer and analyzed with flow cytometry. Stained cells were read on Gallios analyzer (Beckman Coulter) and 20,000 events were counted.

### Example 29

### Growth of Human Ovarian and Breast Cancer Xenografts in Mice

Experiments with Hsd:Athymic nu/nu-Foxn1^{nu} mice (Envigo) were reviewed and approved by the Institutional Animal Care and Use Committee of M. D. Anderson Cancer Center (IACUC 00001052).

### Example 30

### SKOv3 and OVCAR8 Ovarian Cancer Xenografts

Sixty female nu/nu mice were injected with 5×10⁶ SKOv3 cells subcutaneously or 3.5×10⁶ OVCAR8 cells intraperitoneally, respectively. After 7-days, mice were randomly assigned to the following treatment groups (n=10): 1) control vehicle, 2) Compound A (40 mg/kg for SKOv3 or 50 mg/kg for OVCAR8 per mouse, five times per week), 3) Compound B (40 mg/kg for SKOv3 or 50 mg/kg for OVCAR8 per mouse, five times per week), 4) olaparib (50 mg/kg per mouse, five times per week), 5) Compound A combined with olaparib, and 6) Compound B combined with olaparib. All mice were treated orally with vehicle control, single agent or combination of single agents for 4 weeks (SKOv3 xenograft models) or 6 weeks (OVCAR8 xenograft models) and sacrificed with CO₂ one week after completion of treatments. For SKOv3 xenograft models, tumors were measured every week in two dimensions using a digital caliper, and the tumor volume was calculated with the following formula: tumor volume (mm3)=0.5×ab² (a and b being the longest and the shortest diameters of the tumor, respectively). Mice were monitored until tumor burden reached 1500 mm3 (ethical endpoint). For OVCAR8 xenograft models, all tumors were weighed immediately after death.

### Example 31

### OC316 Ovarian Cancer Xenografts

Forty female nu/nu mice were injected with 3.5×10⁶ cells intraperitoneally. After 7-day inoculation, tumor-bearing mice were randomly divided into 4 groups (n=10): 1) control vehicle, 2) Compound B (50 mg/kg five times per week), 3) olaparib (50 mg/kg per mouse, five times per week), 4) Compound B combined with olaparib, and 6) Compound B combined with olaparib. All mice were treated orally with vehicle control, single agent or combination of single agents for 5 week and then continually monitored for survival. Mice were monitored until dyspnea, weight loss, hunched posture, snuffling respiratory sounds or abdominal breathing were observed (ethical endpoint) for euthanasia.

### Example 32

### MDA-MB-231 Breast Cancer Xenografts

Forty female nu/nu mice were injected with 0.8×10⁶ MDA-MB-231 cells into their fourth mammary fat pads. After 7-days, tumor-bearing mice were randomly divided into 4 groups (n=10): 1) control vehicle, 2) Compound B (50 mg/kg five times per week), 3) olaparib (50 mg/kg per mouse, five times per week), 4) Compound B combined with olaparib, and 6) Compound B combined with olaparib. All mice were treated orally with vehicle control, single agent or a combination of single agents for 5 weeks and then continually monitored for survival. Tumors were measured every week as noted above (SKOv3 xenograft models).

### Example 33

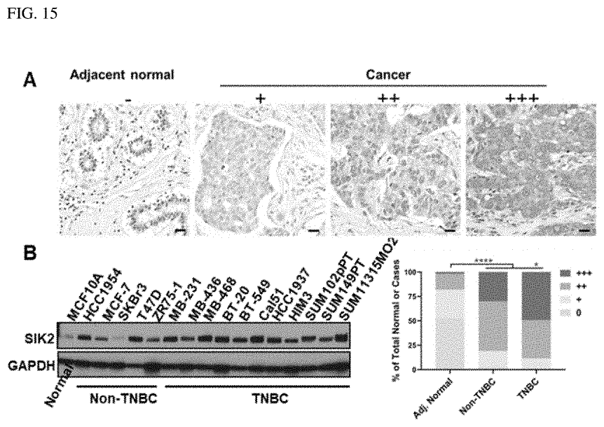

Expression of SIK2 in breast cancers was measured, performing immunohistochemical staining of a tissue microarray (TMA) with 120 non-TNBC cases, 130 TNBC cases and 61 normal and adjacent normal breast tissues. Intense (2-3+) SIK2 staining was observed in 80% of 120 non-TNBCs with lower levels of SIK2 protein (0-1+) in the remaining cancers, compared to intense (2-3+) SIK2 staining in 18% of adjacent normal breast tissue with lower levels of SIK2 protein (0-1+) in the remaining cases (FIG. 15A). More importantly, among 130 TNBCs, 88% exhibited intense staining with lower levels of SIK2 protein in the remaining cases. When SIK2 expression was measured in sixteen breast cancer cell lines, including eleven TNBC cell lines, SIK2 protein expression was significantly increased in the sixteen breast cancer cell lines compared to a normal breast cell line (MCF-10A). SIK2 was highly expressed in 11 of 11 TNBC cell lines (FIG. 15B).

### Example 34

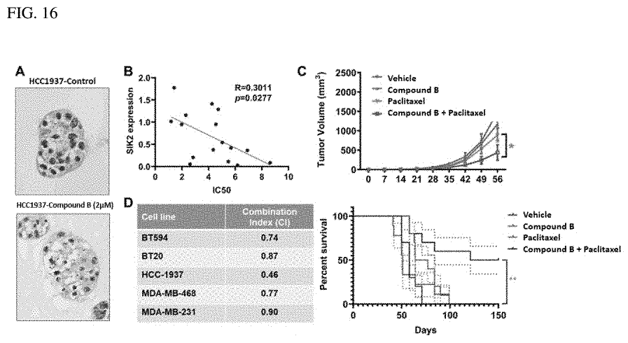

Compound B inhibits cell growth and increases paclitaxel sensitivity in breast cancer cells and xenografts. Growth inhibition was observed in a range of breast cancer cell lines after treatment with Compound B. Those breast cancer cell lines include MCF-7, ZR75-1, BT20, SKBr-3, AU565, MDA-MB -231, MDA-MB-468, MDA-MB-436, HCC1954, HCC1937, SUM1315MO2, BT-549, SUM102PT, SUM149PT, HIM3 and Cal51. The IC50 of Compound B in these breast cancer cell lines ranged from 1.19 to 8.6 μM. Compound B inhibits organoid growth inducing cell death (FIG. 16A). The IC50 of Compound B is inversely correlated with SIK2 protein expression (FIG. 16B) measured by immunoblotting (FIG. 16B). Compound B also inhibited xenograft growth and prolonged the survival of mice bearing MDA-MB-231 orthotopic xenografts (FIG. 16C). To evaluate additive or synergistic interactions, a combination Index (CI value) was calculated with CalcuSyn software. Values less than 1 are considered synergistic and those equal to 1 are considered additive. At the combination index that reflected 90% inhibition, arguably the most relevant metric for cancer treatment, a combination of Compound B and paclitaxel exhibited synergy in 5/5 TNBC cell lines tested (FIG. 16D). These data support use of paclitaxel in combination with Compound B to achieve synergistic cytotoxicity for TNBC.

### Example 35

### A Novel Salt Inducible Kinase 2 Inhibitor, Compound B, Sensitizes Ovarian Cancer Cell Lines and Xenografts to Carboplatin

Salt-induced kinase 2 (SIK2) is a serine-threonine kinase that regulates centrosome splitting, activation of PI3 kinase and phosphorylation of class IIa HDACs, affecting gene expression. Previously, the Inventors found that inhibition of SIK2 enhanced sensitivity of ovarian cancer cells to paclitaxel. Carboplatin and paclitaxel constitute first-line therapy for most patients with ovarian carcinoma, producing a 70% clinical response rate, but curing <20% of patients with advanced disease. The present study studied whether inhibition of SIK2 with Compound B enhances sensitivity to carboplatin in ovarian cancer cell lines and xenograft models. Compound B-induced DNA damage and apoptosis were measured with γ-H2AX accumulation, comet assays, and annexin V. Compound B inhibited growth of eight ovarian cancer cell lines at an IC50 of 0.8 to 3.5 μM. Compound B significantly enhanced sensitivity to carboplatin in seven of eight ovarian cancer cell lines and a carboplatin-resistant cell line tested. Furthermore, Compound B in combination with carboplatin produced greater inhibition of tumor growth than carboplatin alone in SKOv3 and OVCAR8 ovarian cancer xenograft models. Compound B enhanced DNA damage and apoptosis by downregulating expression of survivin. Thus, a SIK2 kinase inhibitor enhanced carboplatin-induced therapy in preclinical models of ovarian cancer and deserves further evaluation in clinical trials.

### Example 36

### Reagents

Compound B was provided by Arrien Pharmaceuticals (U.S. Pat. No. 9,260,426-B2). The purity is 98.2%. The drug was dissolved in DMSO at 10 mM as a stock for in vitro assays. The final concentration of DMSO was <1%. Compound B was dissolved in 5% of ethanol, 30% of polyethylene glycol-300 and 2% of Tween 80 (v/v) by sonication for in vivo animal studies. Carboplatin and paclitaxel were purchased from MD Anderson Pharmacy at 10 mg/mL and 6 mg/mL, respectively. Carboplatin was prepared in sterile water and diluted in culture media for in vitro assays. For in vivo animal studies, drugs were diluted in sterile saline to desired concentrations.

### Example 37

### Cell Lines and Cultures

OVCAR8, SKOv3, OC316, OVCAR3, ES2, A2780, IGROV1, and MDA2774 human ovarian cancer cell lines were provided by UT MD Anderson Cancer Center, Houston, Tex., USA. SKOv3 WT and SKOv3-SIK2 KD (clone 1D) cell lines, OVCAR8 WT and OVCAR8-SIK2 KD (clone 2-3A) cell lines were provided by Oxford University, Oxford, UK. A2780-PAR and A2780-CP20 were kindly provided by MD Anderson. The STR DNA fingerprinting was performed at MD Anderson (Characterized Cell line Core). In addition, mycoplasma was tested in the cell lines using Universal Mycoplasma Detection Kit (ATCC® 30-1012K) and all cell lines were free from contamination. RPMI1640 was used for culturing OVCAR8, SKOv3, OC316, OVCAR5, OVCAR3, ES2, IGROV1, MDA2774, OVCAR8 WT, and OVCAR8-SIK2 KD cells. McCoy's 5A was used for culturing SKOv3 WT and SKOv3-SIK2 KD. Both RPMI1640 and McCoy's 5A were purchased from the Media Preparation Core Facility at MD Anderson Cancer Center.

### Example 38

### Cell Viability Assays

Cells were seeded in 6 replicates in black-walled and clear-bottomed 96-well plates and incubated overnight. Cells then were treated with Compound B and/or carboplatin for an additional 4 days using the concentrations indicated in each figure. The CellTiter-Glo luminescent cell viability assay (Promega) was used to evaluate the effect of treatment on cancer growth. This experiment was performed several times to optimize the concentration. To study the interaction of drugs, the Compound B concentration was reduced incrementally starting from the concentration equal to the IC50 value of Compound B used as a single agent in FIG. 1A. And the concentrations shown above can shift the carboplatin dose-response curve to the left, indicating improved drug responses. GraphPad Prism 8 was used to generate growth curves and calculated IC50. CalcuSyn was used to evaluate additive or synergistic interactions, a combination Index (CI value). Values <1 are considered synergistic and Values >1 or =1 are additive or sub-additive.

### Example 39

### Clonogenic Survival Assays

Cancer cells were seeded in 6-well plates at a density of 400 cells per well in culture medium for 24 h to permit cell adherence. Subsequently, cells were treated with Compound B and/or carboplatin in triplicate. After treatment, cells were grown for an additional 12-14 days. After control colonies had grown to include at least 50 cells, cultures were fixed and stained with Coomassie blue (0.1% Coomassie brilliant blue R-250, 40% methanol, and 10% acetic acid) and counted. Colonies were counted from three independent experiments and the mean number of colonies and standard deviations calculated. Multiplicity adjusted p values for each treatment and control were determined.

### Example 40

### Protein Extraction and Western Blot Analysis

Cells were incubated for 24-48 h with and without treatments and then harvested for western blot analysis. Briefly, cells were incubated in lysis buffer for 20 min on ice and centrifuged at 17,000×g for 10 min at 4° C. Protein concentration of cell lysates was determined with BCA reagent (Thermo Fisher Scientific, Houston, Tex., USA). Lysates were separated on 8-16% SDS-PAGE and transferred to polyvinylidene difluoride (PVDF) membranes. Immunoblots were probed with anti-survivin antibody (Novus; 1:2000, Centennial CO, USA) in 5% BSA overnight at 4° C. and HRP labeled secondary antibody was added for 1 h at RT. The signal was developed on X-ray films.

### Example 41

### Immunofluorescence Staining

Cells were seeded on coverslips in 12-well plates with or without treatment as indicated in each figure. Cells were then fixed in 4% paraformaldehyde (Affymetrix, Sunnyvale, Calif., USA) for 10 min, permeabilized with 0.1% triton X-100 in PBS for 15 min, and then blocked with 1% BSA in PBS for 1 h at RT followed by incubation with anti-r-H2AX at 1:500 dilution (Cell Signaling) at 4° C. overnight. Coverslips were washed 3 times with PBS after primary rabbit antibody incubation and incubated with anti-rabbit Ig secondary antibody conjugated to Alexa 488 for 1 h (Life Technologies, A11070, 1:200, Austin, Tex., USA). Cells were rinsed and the nuclei stained with DAPI (Thermo Fisher, 1 μg/mL). Cells were examined using fluorescence microscopy (Olympus 1×71; Olympus Corporation of the Americas, Center Valley, Pa., USA).

### Example 42

### Comet Assays

The OxiSelect™ Comet Assay Kit (Cell Biolabs, Inc., San Diego, Calif., USA) was used to evaluate DNA damage with or without drug treatment. Briefly, cells (1×10⁵ cells/mL) were mixed with molten agarose (Cell Biolabs, Inc.) at 37° C. at a ratio of 1:10 (v/v), and then transferred to comet slides and incubated in the dark for 15 min. Slides were immersed in pre-chilled lysis buffer for 1 h and then with freshly prepared pre-chilled Alkaline Solution (pH>13) for 30 min at 4° C. in the dark. Slides were electrophoresed in alkaline buffer at 1 volt/cm for 30 min. The cells were stained with 1× Vista Green DNA Dye for 15 min at RT and then viewed with a 4× fluorescence microscope. The percentage of DNA in the tail were analyzed with Casplab_1.2.3b2 (CaspLab Comet Assay Software, CASPLab, casplab.com). At least 50 randomly selected cells were analyzed per sample.

### Example 43

### Apoptosis

Assays Cells were grown in 6-well plates at a density of 8×10⁴ cells/plate and treated with or without Compound B and/or carboplatin. After completion of incubation, cells were harvested and washed with PBS two times. After washing, cells were re-suspended in 100 μL Annexin-binding buffer containing propidium iodide (PI) and FITC Annexin-V (Invitrogen) and incubated at ambient temperature for 15 min in the dark. After incubation, 200 μL of Annexin-binding buffer was added and stained cells were analyzed using a Beckman Coulter's Gallios Flow Cytometer.

### Example 44

### Murine Xenografts

Six-week-old female athymic nu/nu mice were purchased from Envigo. Experiments were reviewed and all procedures were performed according to an animal protocol approved by the Institutional Animal Care and Use Committee of UT MD Anderson Cancer Center. OVCAR8 ovarian cancer cells (3×10⁶) were inoculated i.p. and SKOv3 ovarian cancer cells (5×10⁶) were inoculated s.c. For OVCAR8 xenograft models, Compound B was administrated p.o. at a dose of 50 mg/kg/day for 5 days/week for three weeks. Carboplatin was administrated i.p. once a week at a dose of 25 mg/kg. On day 7 after cancer cell injection, mice were randomly assigned to the following treatment groups (n=10 mice per group): (1) non-treatment diluent control; (2) Compound B; (3) carboplatin; and (4) a combination of carboplatin and Compound B. For SKOv3 xenograft models, Compound B was treated with at a dose of 40 mg/kg/day for six weeks. Carboplatin was treated at a dose of 30 mg/kg once a week for six weeks. Additionally, paclitaxel was administrated i.p. once a week at a dose of 0.8 mg/kg for once a week for six weeks. One week after cells injection, ten mice per group were randomly assigned to the following six groups: (1) diluent control; (2) Compound B; (3) carboplatin; (4) paclitaxel; (5) a combination of carboplatin and Compound B; (6) a combination of paclitaxel and Compound B; (7) a combination of carboplatin and paclitaxel; and (8) a combination of Compound B, carboplatin and paclitaxel. At end of six weeks, the mice were sacrificed by CO₂. The mice were dissected immediately after death and the tumors were collected and weighed.

### Example 45

### Statistical Analysis

If not stated otherwise, all experiments were set up as triplicates and repeated independently at least twice and the data were expressed as the mean±the standard deviation. GraphPad Prism (version 8.0) was used for plotting and statistical analyses. The two-tailed Student t test (2 groups with unequal variances) and one-way ANOVA for multiple comparisons were performed. The differences at p<0.05 was considered statistically significant.

### Example 46

### Compound B Inhibits Cell Growth and Increases Sensitivity to Carboplatin in Ovarian Cancer Cells

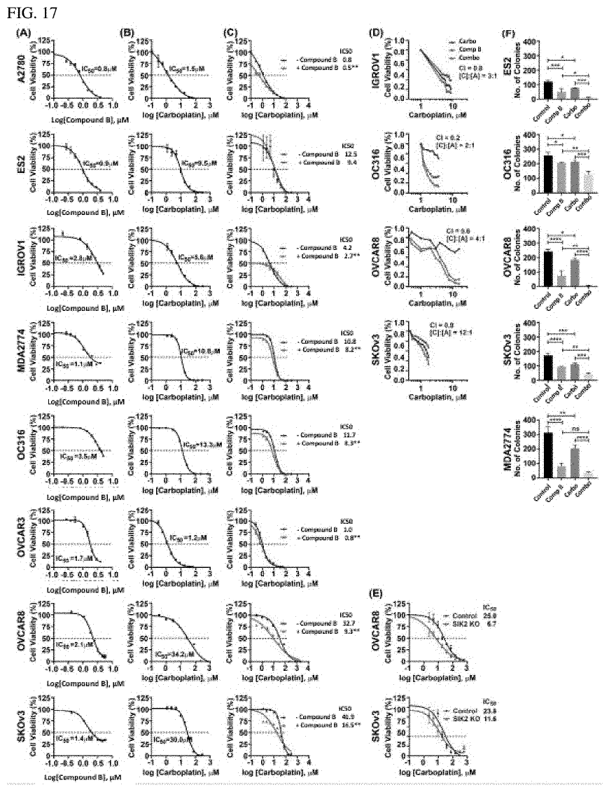

To determine whether Compound B could inhibit the growth of ovarian cancer cells, the effect of Compound B was measured in eight ovarian cancer cell lines using short term cell proliferation assays. The IC50 of Compound B was calculated for each cell line (FIG. 17A). Significant inhibition was achieved in all cell lines in a dose dependent manner. The IC50 values of Compound B for OVCAR8, SKOv3, OC316, OVCAR3, ES2, A2780, MDA2774, and IGROV1 cells ranged from 0.8 to 3.5 μM. The IC50 values for carboplatin with the same ovarian cancer cell lines ranged from 1.2 to 34.2 μM (FIG. 17B). When Compound B was added to carboplatin, the carboplatin dose response curve was shifted to the left in seven of eight ovarian cancer cell lines (FIG. 17C), indicating that Compound B sensitizes ovarian cancer cells to carboplatin (p<0.01). To test whether the interaction was additive or synergistic, multi-point drug combination studies were conducted in four of the most responsive cell lines (IGROV1, OC316, OVCAR8, and SKOv3), and calculated a combination index (CI) using the Chou-Talaley method, based on a median-effect equation to define the drug response to the combination quantitatively. CI values in response to the Compound B and carboplatin combination were less than one in all four cell lines (FIG. 17D and Table 3), supporting the hypothesis that SIK2 inhibition enhances sensitivity to carboplatin.

TABLE 3
The Combinatorial effect of carboplatin and Compound B

| | Combination Ratio | Combination Index at |
|---|---|---|
| | [Carbo]:[COMPOUND B] | The Effect Level of 95% |
| IGROV1 | 3:1 | 0.75 |
| OC316 | 2:1 | 0.19 |
| OVCAR8 | 4:1 | 0.59 |
| SKOv3 | 12:1 | 0.92 |

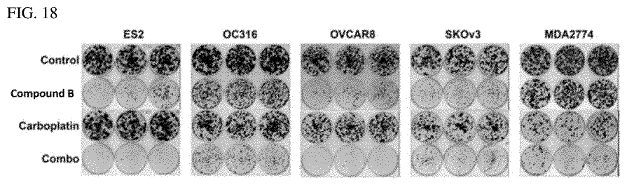

Furthermore, to exclude potential off-target effects of Compound B, SIK2 was knocked down with CRISPR/Cas9 in OVCAR8 and SKOv3 ovarian cancer cells. Knockout of SIK2 sensitized ovarian cancer cells to carboplatin in a manner similar to Compound B (FIG. 17E). In addition, five cell lines were used to compare the effect of Compound B and carboplatin to either agent alone using clonogenic assays, which showed that Compound B significantly enhanced carboplatin induced loss of clonogenic survival in the OVCAR8, SKOv3, OC316, MDA4772 and ES2 ovarian cancer cell lines (FIG. 17F and FIG. 18). Taken together, these data suggest that the inhibition of SIK2 kinase activity potentiates carboplatin in ovarian cancer cells, and Compound B, a potent SIK2 selective inhibitor, works synergistically with carboplatin to kill ovarian cancer cells.

### Example 47

### Compound B or SIK2 Knockout Enhances Carboplatin-Induced Apoptosis by Downregulating Survivin

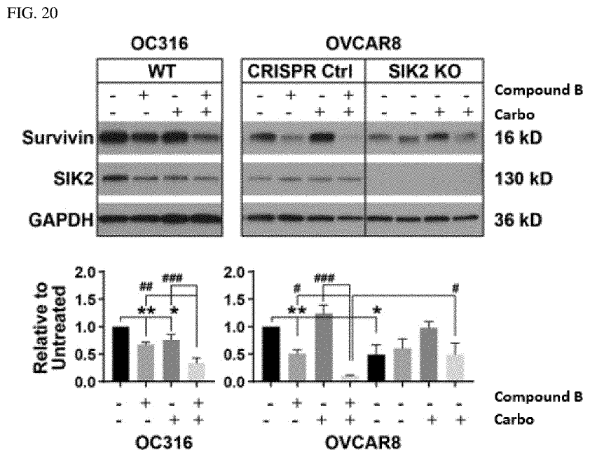

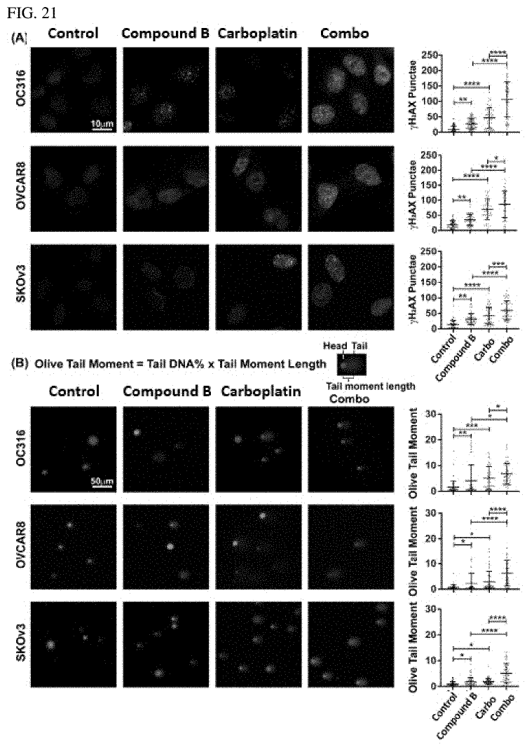

Many current cancer chemotherapies, including platinum-based drugs, exert their antitumor effect by triggering apoptosis in cancer cells. To study the underlying mechanism of SIK2 inhibition-induced carboplatin-mediated cell toxicity, apoptosis was measured using flow cytometry in OVCAR8, SKOv3, and OC316 ovarian cancer cell lines. Compound B not only induced apoptosis as a single agent, but also enhanced carboplatin-induced apoptosis (FIG. 20A). In addition, a similar effect was observed in SIK2 knockout cell lines (OVCAR8 and SKOv3) showing that abolishing the function of SIK2 enhanced ovarian cancer cells to carboplatin-mediated apoptosis (FIG. 20B). Together, the data suggest that SIK2 inhibition enhances carboplatin sensitivity by increasing carboplatin-induced apoptotic cell death. The inhibitor of apoptosis protein family (IAPs) includes an important group of proteins involved in the regulation of apoptosis. One member of this protein family, survivin, plays an important role in promoting tumor progression by deregulating apoptosis and cell division. In the present study, downregulation of survivin was observed with Compound B and greater downregulation was observed with the combination of the two drugs in OC316 and OVCAR8 ovarian cancer cell lines (FIG. 21). When SIK2 was knocked out using CRISPR/cas9, cells expressed survivin and the downregulation phenotype with Compound B treatment was partly reversed (FIG. 21, right panel). Thus, downregulation of survivin and a consequent activation of apoptosis could contribute to Compound B-mediated carboplatin sensitization.

### Example 48

### Compound B Enhances Carboplatin-Induced DNA Damage

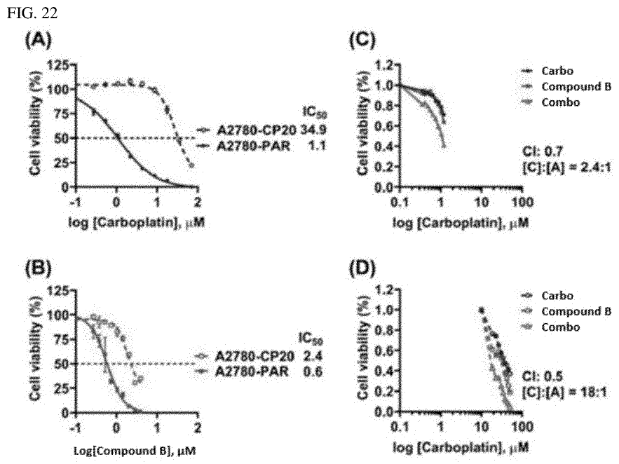

The biochemical mechanism(s) for cytotoxicity of cisplatin and carboplatin involve covalent binding to DNA and induction of cell death through apoptosis within the heterogeneous population of tumor cells. Direct binding of platinum-based drugs to genomic DNA in cancer cells can result in a number of lesions including bulky platinum-DNA adducts and DNA double-strand breaks (DSBs). Detection of increased γ-H2AX punctae is an early and sensitive indicator of DSBs after treatment with cisplatin or carboplatin in cancer cells. As carboplatin induces apoptosis, it was examined whether treatment with Compound B increases carboplatin-induced γ-H2AX punctae in OVCAR8, SKOv3, and OC316 ovarian cancer cell lines. Compound B and carboplatin showed a greater increase in γ-H2AX punctae than either agent alone (FIG. 22A). In addition, a comet assay was also performed to measure DNA damage after treatment with Compound B, carboplatin, or the combination of two drugs. Consistent with an increase in γ-H2AX punctae, the comet tail moment induced with carboplatin was further enhanced by Compound B (FIG. 22B). Thus, it suggests that Compound B enhances carboplatin-mediated apoptosis by increasing carboplatin-induced DNA damage.

### Example 49

### Compound B Inhibits Growth of Cisplatin-Resistant Cancer Cell Lines and Enhances Sensitivity to Carboplatin

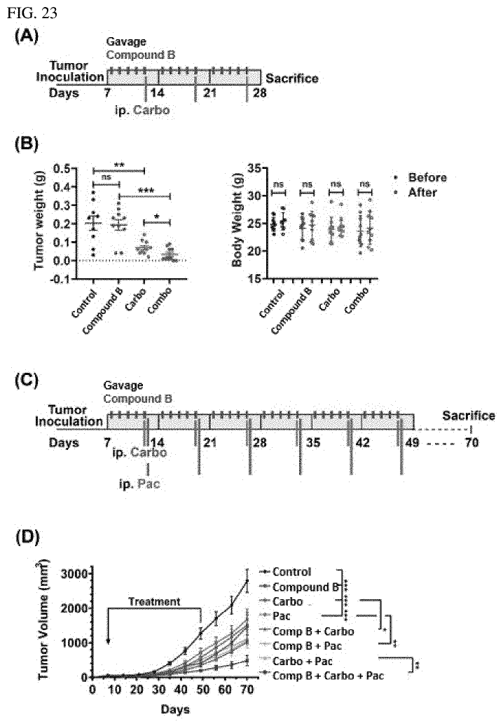

Platinum resistance is commonly seen in ovarian cancer patients with recurrent disease. There is currently no standard chemotherapy for platinum-resistant recurrence. Targeting DNA damage and repair is an attractive therapeutic approach in platinum-resistant ovarian cancer. As Compound B enhances carboplatin-induced DNA damage, it was tested whether SIK2 inhibition with Compound B would overcome platinum-induced resistance in ovarian cancer cells. A2780-PAR cisplatin-sensitive and A2780-CP20 cisplatin-resistant ovarian cancer cells were tested for carboplatin response and the IC50 of A2780-CP20 (34.9 μM) was 32-fold higher than IC50 of A2780-PAR (1.1 μM) (FIG. 23A). Compound B inhibited both the resistant and sensitive cell lines in a dose dependent fashion with IC50's of 2.4 and 0.6 μM, respectively (FIG. 23B). Treatment with the combination provided synergistic enhancement of the carboplatin effect as CI values were <1 (FIG. 22C-FIG. 22D). Thus, Compound B enhanced sensitivity to carboplatin not only in carboplatin-sensitive ovarian cancer cells but also in carboplatin-resistant ovarian cancer cells.

### Example 50

### Compound B Enhances the Activity of Carboplatin in Human Ovarian Cancer Xenograft Models

Given the synergistic effect of Compound B and carboplatin in inhibiting the growth of cultured ovarian cancer cells, it was investigated whether the addition of the SIK2 inhibitor could promote carboplatin response in xenograft models. OVCAR8 cells were injected intraperitoneally (ip) into nu/nu mice. Treatment started 7 days post injection. Compound B (50 mg/kg) was administered orally five days a week while carboplatin (25 mg/kg) was injected i.p. once a week for three weeks. At the conclusion of the treatment, the mice were sacrificed, and the tumor was dissected and weighed (FIG. 23A). Treatment with Compound B significantly enhanced the growth inhibitory effect of carboplatin (p<0.05) (FIG. 23B). Moreover, the combination of Compound B with carboplatin was well tolerated, with no significant weight loss compared to vehicle control (FIG. 23B). To validate results observed in the OVCAR8 xenograft model, SKOv3 cells were injected subcutaneously into nu/nu mice. Seven days after tumor cell injection, mice were treated with either vehicle, single-agent Compound B (40 mg/kg), carboplatin (10 mg/kg), or paclitaxel (50 mg/kg), or the combination of two or three drugs as indicated for a total of 6 weeks (FIG. 23C). The tumor volume was measured at indicated time points (FIG. 23D). Treatment with Compound B, carboplatin, or paclitaxel alone significantly inhibited tumor growth (p<0.001) (FIG. 23D), compared to vehicle control. The combination of Compound B plus carboplatin (p<0.05) or Compound B plus paclitaxel (p<0.01) produced greater inhibition of tumor growth than either single agent (FIG. 23D). More importantly, Compound B further enhanced the combination treatment of carboplatin plus paclitaxel (p<0.01) (FIG. 23D) which is standard first-line chemotherapy for patients with ovarian cancer.

### Example 51

### Discussion

In this report, it was found that Compound B induces double strand breaks (DSBs) in cancer cell DNA and produces synthetic lethality with carboplatin. SIK2 is an AMP activated protein kinase that is required for ovarian cancer cell proliferation and metastasis. SIK2 is overexpressed in 30% of ovarian cancers, correlating with poor prognosis in patients with high-grade serous ovarian carcinomas. Compound B inhibited cancer cell growth in eight ovarian cancer cell lines with IC50 concentrations that ranged from 0.8 to 3.5 μM. Compound B enhanced carboplatin sensitivity in seven of eight ovarian cancer cell lines and in two xenograft models. Compound B significantly increased carboplatin-mediated γ-H2AX production and DNA comet tail moment, indicating enhanced DNA damage and/or decreased DNA repair. In addition, treatment with Compound B sensitized both a relatively sensitive A2780-parental cell line and the highly resistant A2780-CP20 cell line, demonstrating that Compound B enhanced sensitivity to carboplatin both in carboplatin-sensitive and in carboplatin-resistant ovarian cancer cells.

Platinum-based drugs including cisplatin, carboplatin, and oxaliplatin are widely used for the treatment of different cancers. Treatment with a combination of paclitaxel and carboplatin is considered first line therapy for advanced ovarian cancer. Ovarian cancer responds well to both cisplatin and carboplatin, but after an initial response, the majority of patients with ovarian cancer will relapse and develop the resistance. Because the main target of platinum drugs is DNA, the sensitivity and resistance to those drugs is associated with the ability of cells to repair the platinum-induced DNA damage. Compound B was found to enhance carboplatin-induced DNA damage judged by γ-H2AX accumulation and an increase in comet assay tail moment. Enhancement of DNA damage was associated with an increase in apoptosis that was most pronounced with the combination of Compound B and carboplatin. This combination also downregulated survivin. Recent studies show survivin is associated with both inhibiting apoptosis and regulating cell mitosis in cancer. Survivin overexpression has been shown to correlate with chemo-resistance in several cancers. Several molecular approaches that downregulating survivin expression and/or block its function are being developed in the clinic. The past and present findings indicate that the SIK2 inhibitor Compound B enhances sensitivity to both carboplatin and paclitaxel in cultured ovarian cancer cell lines as well as in xenograft models, supporting its potential role in the treatment of primary as well as recurrent ovarian cancer.

Taken together, these studies encourage the further clinical evaluation of Compound B. A phase I study of Compound B alone and in combination with paclitaxel is underway. Pre-clinical toxicology studies indicate that treatment with Compound B has little effect on normal hematopoietic or organ function. In the present study, treatment with Compound B and carboplatin did not affect the body weight of nude mice.

SIK2 inhibitor Compound B enhances sensitivity to carboplatin of both carboplatin-sensitive and -resistant ovarian cancer cells in vitro, inhibits tumor xenograft growth and enhances sensitivity to both carboplatin and paclitaxel in in vivo xenograft models.

### Example 52

### Discovery of Compound B as a Potent, Selective, Orally Available SIK2 Inhibitor for Treating Ovarian, Endometrial, Primary Peritoneal, Fallopian Tube, and Triple-Negative Breast Cancers

Compound B is an orally bioavailable small molecule inhibitor of the Salt Inducible Kinase 2 (SIK2, 11 nM) and SIK3 (19 nM). Three isoforms of SIK (SIKs) proteins have been reported: SIK1 (SNF1LK), SIK2 (QIK), and SIK3 (QSK). They are the Ser/Thr centrosome kinase family members required for bipolar mitotic spindle formation. The overexpression of SIK2 kinase in 30% of ovarian cancer specimens allows a novel, clinically important new method of treating ovarian cancer by blocking SIK2 kinase activity. In addition to a role in ovarian cancer, SIK2 and SIK3 are prevalent in several other tumor types, including breast, prostate, diffuse large B-cell lymphoma, and melanoma cancers. SIK2 has been reported to cause centrosome splitting in interphase, while SIK2 depletion blocked centrosome separation in mitosis and sensitized ovarian cancers to paclitaxel in culture and in vivo xenograft models. Depletion of SIK2 also delayed G1/S transition and reduced AKT phosphorylation. Higher levels of expression of SIK2 have been shown to be highly correlated with poor survival in patients with high-grade serous ovarian cancers. Using the homology structure of SIK2, fragment-based lead optimization strategies, and screening and structure-activity relationship efforts, it was discovered Compound B, a first-in-class novel, selective inhibitor of SIK2 that could prove useful in treating ovarian, endometrial, primary peritoneal, fallopian tube, and triple negative breast cancers. Compound B specifically inhibited SIK2-expressed SKOv3 cells with an IC50 of 92 nM. Compound B was effective against ovarian, breast cancer cell lines alone and in combination with paclitaxel and cisplatin. Compound B also inhibited ovarian tumor growth significantly at 70% in SKOv3 human ovarian cancer xenografts in Ncr nu/nu mice in a dose dependent manner at 20, 40, 60, and 100 mg/kg orally. Moreover, Compound B has exhibited excellent in vivo pharmacokinetic, pharmacodynamics, and correlative PK/PD and ADME characteristics. Preliminary in vitro and in vivo tumor up-take studies suggest that Compound B blocks centrosome separation by inhibiting SIK2, thereby enhancing the sensitivity of paclitaxel.

SIKs Signaling and Compound B: Salt Inducible Kinase 2 (SIK2) is a centrosome kinase, (a member of AMPK family of kinases), which is required for bipolar mitotic spindle formation and is a Ser/Thr kinase. Three isoforms of SIK family have been reported; SIK1 (SIK, SNF1LK), SIK2 (SNF1LK, QIK), and SIK3 (QSK). Compound B potently inhibits SIK2 and SIK3 (Table 4).

Results:

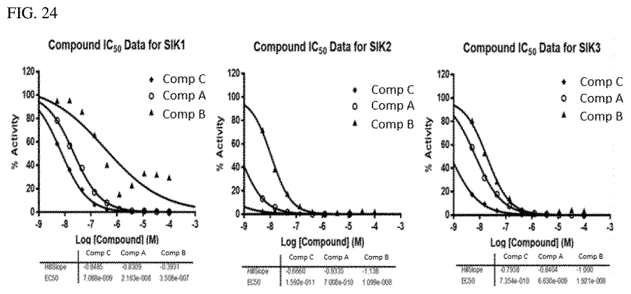

The dose response curve for Compound B and its analogues against SIK isoforms is shown in FIG. 24 and Table 4. Compound B is a first-in-class novel inhibitor of SIK2 that would be useful for treating ovarian, endometrial, primary peritoneal, fallopian tube, and triple negative breast cancers.

TABLE 4

| Kinase | Compound A | Compound B | Compound C |
|---|---|---|---|
| SIK1 | 21.63 nM | 350.8 nM | 7.088 nM |
| SIK2 | <1.0 nM | 14.18 nM | <1.0 nM |
| SIK3 | 6.63 nM | 24.53 nM | 0.7354 nM |

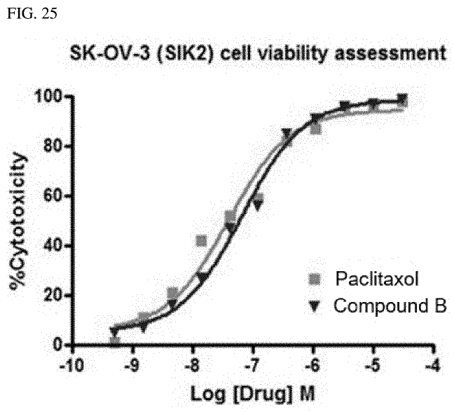

The effects of Compound B on the SIK2-expressed SKOV3 cells, Cell viability assessment is shown in FIG. 25 and Table 5.

TABLE 5

| | | | | | # of points used |
|---|---|---|---|---|---|
| Compound ID | IC50 | Hillslope | Z′ | Constraint | for DRC |
| Paclitaxel | 39 nM | 0.85 | 0.9 | — | 11 |
| Compound B | 62 nM | 0.83 | 0.9 | — | 11 |

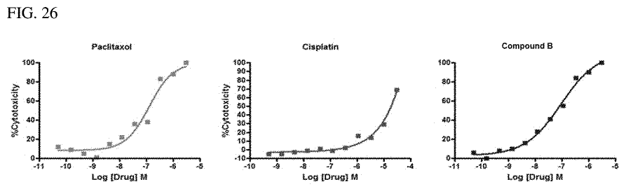

A cell viability assessment of SKOV3 on paclitaxel, cisplatin, and Compound B treatment is shown in FIG. 26. and Table 6.

TABLE 6

| | | | | | | # of points used |
|---|---|---|---|---|---|---|
| Compound ID | IC50 | | Hillslope | Z′ | Constraint | for DRC |
| Paclitaxel | 127 | nM | 0.98 | 0.7 | — | 11 |
| Cisplatin | 17 | μM | 0.59 | 0.7 | Top locked | 11 |
| | | | | | at 100 | |
| Compound B | 92.4 | nM | 0.64 | 0.7 | — | 11 |

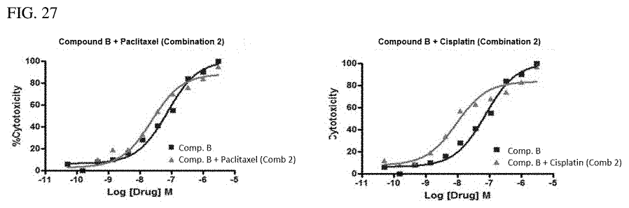

Compound B and paclitaxel combinational effect on SK-OV-3 cell viability is shown in FIG. 27.

The combinational effect of Compound B, paclitaxel, and cisplatin on SK-OV-3 cell viability is shown in Tables 7-10.

TABLE 7
IC50 (nM)

| Com- | Com- | Com- | Com- | | |
|---|---|---|---|---|---|
| pound B + | pound B + | pound B + | pound B + | | |
| Paclitaxel | Paclitaxel | Paclitaxel | Paclitaxel | Compound | |
| (Comb 1) | (Comb 2) | (Comb 3) | (Comb 4) | B | Paclitaxel |
| 53.1 | 23.3 | 47.5 | 35.5 | 92.4 | 126.7 |

TABLE 8
CI50 (Combination Index Value at 50% Cytotoxicity

| Compound B + | Compound B + | Compound B + | Compound B + |
|---|---|---|---|
| Paclitaxel | Paclitaxel | Paclitaxel | Paclitaxel |
| (Comb 1) | (Comb 2) | (Comb 3) | (Comb 4) |
| 1 | 0.4 | 1 | 0.7 |
| | Effect | CI Value | |
| | Synergy | <0.9 | |
| | Additive | 1 | |
| | Antagonist | >1 | |

TABLE 9
IC50 (nM)

| Com- | Com- | Com- | Com- | | |
|---|---|---|---|---|---|
| pound B + | pound B + | pound B + | pound B + | | |
| Cisplatin | Cisplatin | Cisplatin | Cisplatin | Compound | |
| (Comb 1) | (Comb 2) | (Comb 3) | (Comb 4) | B | Cisplatin |
| 34.3 | 9.2 | 16.6 | 21.3 | 92.4 | 16800 |

TABLE 10
CI50 (Combination Index Value at 50% Cytotoxicity

| Compound B + | Compound B + | Compound B + | Compound B + |
|---|---|---|---|
| Cisplatin | Cisplatin | Cisplatin | Cisplatin |
| (Comb 1) | (Comb 2) | (Comb 3) | (Comb 4) |
| 0.4 | 0.1 | 0.2 | 0.2 |
| | Effect | CI Value | |
| | Synergy | <0.9 | |
| | Additive | 1 | |
| | Antagonist | >1 | |

Combinational treatment of SKOV3 cells with Compound B, paclitaxel, and cisplatin was found to have a synergistic cytotoxic effect in the nanomolar range.

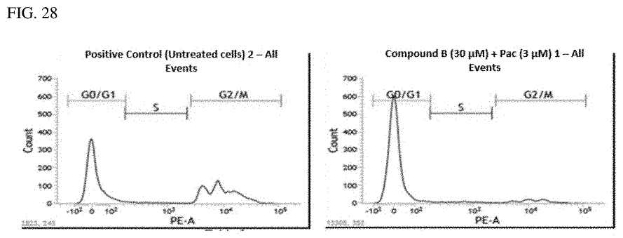

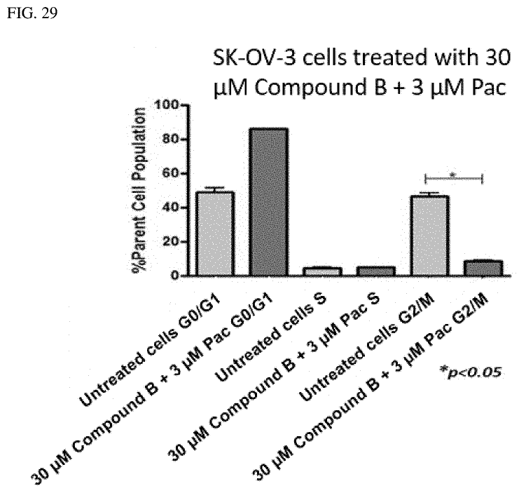

The combination effect of Compound B and paclitaxel on SK-OV-3 cell cycle is shown in FIG. 28 and FIG. 29, as well as Table 11.

TABLE 11

| | | 30 μM Compound B + |
|---|---|---|
| Mean ± SD | Untreated Cells | 3 μM Paclitaxel Treated Cells |
| G0/G1 Phase | 49 ± 4 | 86 ± 0.1 |
| S Phase | 4 ± 1 | 5 ± 0.2 |
| G2/M Phase | 46 ± 3 | 9 ± 0.1 |

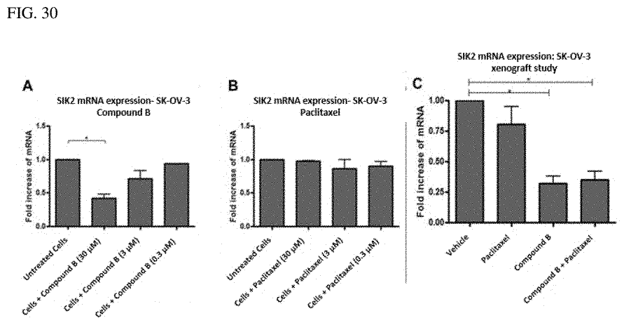

The effect of Compound B and paclitaxel on SIK2 mRNA expression in SK-OV-3 xenograft tumor samples is shown in FIG. 30.

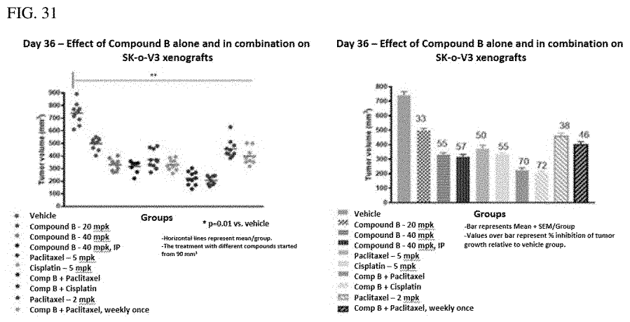

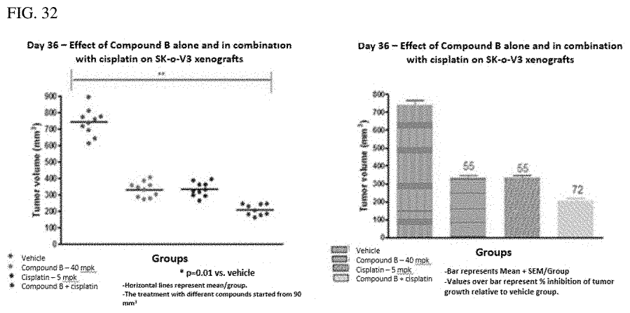

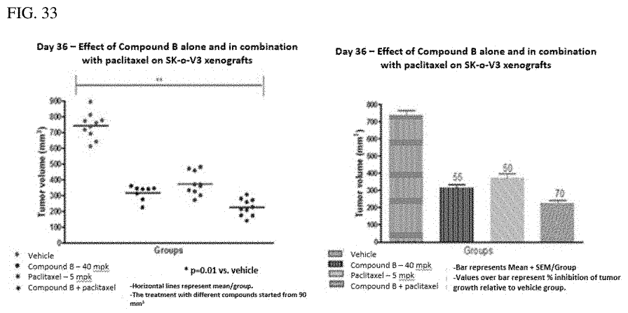

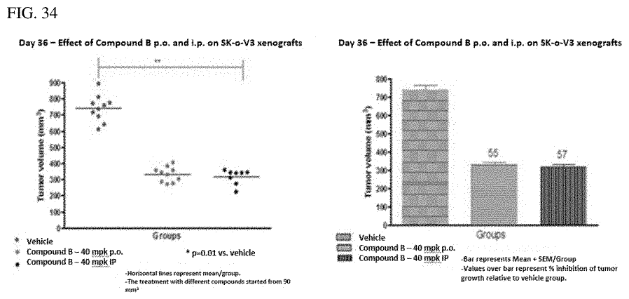

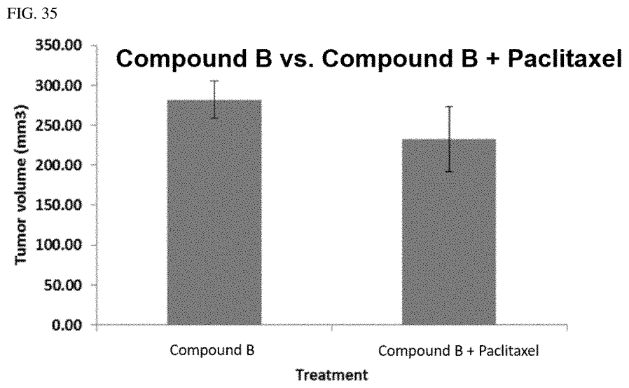

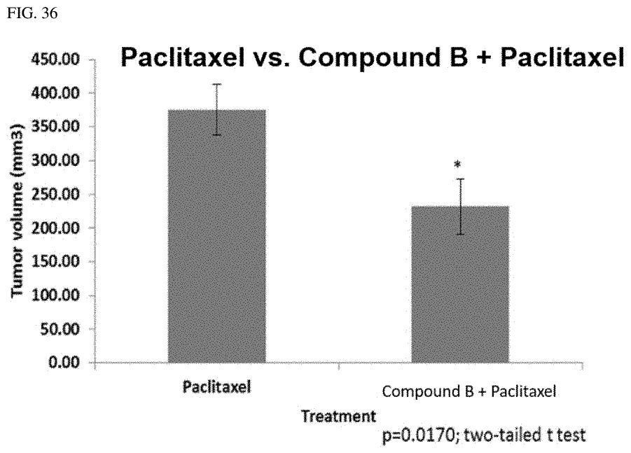

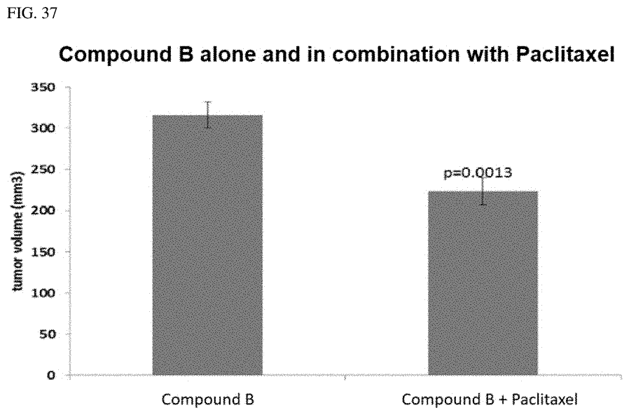

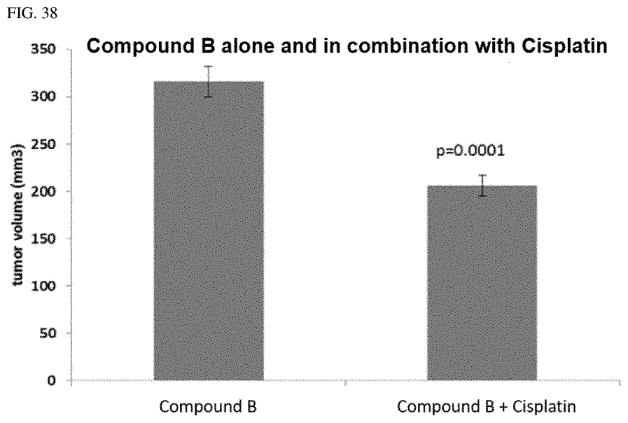

The anti-tumor efficacy of single agent Compound B, in combination with paclitaxel and cisplatin in SK-O-v3 human ovarian tumor xenograft in female nu/nu mice is shown in FIG. 31-FIG. 38.

In-vitro efficacy of Compound B in 13 human breast cancer cell lines using the CellTiter-Blue® Cell Viability Assay is shown in (Table 12).

TABLE 12

| | | Test/Control (%) at drug | | |
|---|---|---|---|---|
| | | Concentration [μM] | | |
| Compound B/Cell Line | | 1.0E+00 | 1.0E+01 | 1.0E+02 |
| MAXF | 401 | 104 | 79 | 52 |
| MAXF | BT-474 | 91 | 78 | 52 |
| MAXF | BT-549 | 88 | 54 | 27 |
| MAXF | Hs 578T | 98 | 40 | 5 |
| MAXF | MCF7 | 92 | 76 | 59 |
| MAXF | MCF 10A | 121 | 116 | 74 |
| MAXF | MDA-MB-231 | 86 | 56 | 42 |
| MAXF | MDA-MB-453 | 80 | 56 | 33 |
| MAXF | MDA-MB-468 | 104 | 87 | 43 |
| MAXF | MX1 | 127 | 118 | 114 |
| MAXF | SK-BR-3 | 106 | 95 | 72 |
| MAXF | T47D | 89 | 76 | 62 |
| MAXF | ZR-75-1 | 100 | 100 | 94 |

The effect of Compound B and Paclitaxel on TNBC cells is shown in Table 13 and Table 14.

TABLE 13

| Compound B/ | | Test/Control (%) at drug Concentration [μM] | | | | | | | | |
|---|---|---|---|---|---|---|---|---|---|---|
| Cell Line | 3.2E−03 | 1.0E−02 | 3.2E−03 | 1.0E−01 | 3.2E−01 | 1.0E+00 | 3.2E+00 | 1.0E+01 | 3.2E+01 | 1.0E+02 |
| MAXF Hs 578T | 101 | 101 | 99 | 97 | 98 | 97 | 92 | 64 | 36 | 19 |
| MAXF | 102 | 104 | 105 | 103 | 103 | 103 | 95 | 80 | 77 | 40 |
| MDA-MB-231 | | | | | | | | | | |

TABLE 14

| Paclitaxel/ | | Test/Control (%) at drug Concentration [μM] | | | | | | | | |
|---|---|---|---|---|---|---|---|---|---|---|
| Cell Line | 3.2E−05 | 1.0E−04 | 3.2E−04 | 1.0E−03 | 3.2E−03 | 1.0E−02 | 3.2E−02 | 1.0E−01 | 3.2E−01 | 1.0E+00 |
| MAXF Hs 578T | 98 | 101 | 99 | 104 | 100 | 89 | 52 | 44 | 42 | 38 |
| MAXF | 101 | 108 | 108 | 110 | 101 | 45 | 24 | 17 | 14 | 10 |
| MDA-MB-231 | | | | | | | | | | |

The data indicates that Compound B is safe and efficacious. Compound B on target SIK2 activity may further serve as an effective therapeutic agent in ovarian, endometrial, primary peritoneal, fallopian tube, and triple negative breast cancer patients.

1. A method of treating ovarian cancer in a patient in need thereof, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

2. A method of increasing or enhancing apoptosis of ovarian cancer cells in a patient having ovarian cancer, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or a SIK2 inhibitor and a combination of paclitaxel, cisplatin, and carboplatin.

3. (canceled)

4. (canceled)

5. A method of increasing or enhancing carboplatin-induced DNA damage in a patient having ovarian cancer, comprising administering to the patient therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

6. (canceled)

7. (canceled)

8. A method of suppressing tumor growth in a cancer patient in need thereof, comprising administering to the patient in need thereof therapeutically effective amounts of:
a SIK2 inhibitor and carboplatin; or
a SIK2 inhibitor and a combination of paclitaxel and cisplatin; or
a SIK2 inhibitor and a combination of paclitaxel and carboplatin; or
a SIK2 inhibitor and a combination of paclitaxel, cisplatin, and carboplatin.

9. The method of claim 1, further comprising at least an additional chemotherapeutic drug.

10. The method of claim 1, wherein the cancer is ovarian, endometrial, primary peritoneal, fallopian tube, and breast cancer.

11. The method of claim 10, wherein the ovarian cancer is primary or recurrent.

12. The method of claim 11, wherein the ovarian cancer is carboplatin-sensitive or carboplatin-resistant ovarian cancer.

13. (canceled)

14. The method of claim 10, wherein the ovarian cancer is high-grade serous ovarian carcinoma (HGSOC).

15. The method of claim 1, wherein the SIK2 inhibitor and the combination of carboplatin and paclitaxel results in a 70% clinical response.

16. The method of claim 9, wherein the combination of:
the SIK2 inhibitor and carboplatin; or
the SIK2 inhibitor and a combination of paclitaxel and cisplatin; or
the SIK2 inhibitor and a combination of paclitaxel and carboplatin; or
the SIK2 inhibitor and a combination of paclitaxel, cisplatin, and carboplatin;
inhibits growth of ovarian cancer cells; and/or
induces increased or enhanced levels of apoptosis in the cancer cells compared to cancer cells treated with only the SIK2 inhibitor, or with only the carboplatin, or with only the combination of paclitaxel and cisplatin, or with only the combination of paclitaxel and carboplatin, or with only the combination of paclitaxel, cisplatin, and carboplatin; and/or
enhances sensitivity of the cancer cells to the chemotherapeutic drug; and/or
produces a synergistic growth inhibition of the cancer cells; and/or
decreases expression of one or more genes involved in regulation of DNA repair and apoptosis in the cancer cell compared to cells treated with the SIK2 inhibitor or the carboplatin or the combination of paclitaxel and cisplatin; or the combination of paclitaxel and carboplatin; or the combination of paclitaxel, cisplatin, and carboplatin alone.

17. The method of claim 10, wherein the breast cancer is triple-negative breast cancer.

18. The method of claim 1, wherein the SIK2 inhibitor is Compound B.

19. The method of claim 1, wherein the SIK2 inhibitor is administered orally.

20. The method of claim 1, wherein the SIK2 inhibitor blocks DNA double-strand break (DSB) repair in the cancer cells.

21. The method of claim 20, wherein the SIK2 inhibitor blocks DNA DSB repair by increasing nuclear localization of histone deacetylase (HDAC) 4/5, wherein the increased nuclear localization of HDAC4/5 blocks the activity of transcription factors associated with DNA DSB repair.

22. The method of claim 21, wherein the transcription factor associated with DNA DSB repair is a myocyte enhancer factor-2 (MEF2) protein.

23. The method of claim 22, wherein the MEF2 protein is MEF2D.

24. (canceled)

25. The method of claim 16, wherein the increased level of apoptosis in the cancer cells is the result of an increase in DNA damage and a decrease in the levels of survivin in the cancer cell.

26. (canceled)

27. (canceled)

28. (canceled)

29. The method of claim 16, wherein the one or more genes involved in regulation of DNA repair and apoptosis in the cancer cell are selected from BRCA2, EXO1, FANCD2, LIG4, XRCC4, BAX, BCL2, CASP7, and TRADD.

30. The method of claim 29, wherein the one or more genes involved in regulation of DNA repair and apoptosis in the cancer cell are selected from EXO1, FANCD2, and XRCC4.

31. The method of claim 29, wherein expression of the one or more genes is decreased by decreasing MEF2D binding to promoter regions.

32. The method of claim 1, wherein the ovarian cancer is platinum-resistant ovarian cancer.
